(19) United States
(12) Patent Application Publication
(10) Pub. No.: US 2005/0277676 A1
(43) Pub. Date: Dec. 15, 2005

(54) M3muscarinic acetylcholine receptor antagonists

(76) Inventors: Dramane I. Laine, King of Prussia, PA (US); Richard Bell, Stevenage (GB); Jakob Busch-Petersen, King of Prussia, PA (US); Michael R. Palovich, King of Prussia, PA (US)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/523,478

(22) PCT Filed: Aug. 6, 2003
(86) PCT No.: PCT/US03/24569

§ 371 Date: Feb. 4, 2005

(30) Foreign Application Priority Data
Aug. 6, 2002 (US) 60401756

(51) Int. Cl. A61K 31/454; C07D 417/02

(52) U.S. Cl. 514/326; 546/208

(57) ABSTRACT

M₃ Muscarinic Acetylcholine Receptor Antagonists and methods of using them are provided.

### FIELD OF THE INVEIION

This invention relates to novel thiazole aniline compounds, pharmaceutical compositions, processes for their preparation, and use thereof in treating M₃ muscarinic acetylcholine receptor mediated diseases.

### BACKGROUND OF THE INVENTION

Acetylcholine released from cholinergic neurons in the peripheral and central nervous systems affects many different biological processes through interaction with two major classes of acetylcholine receptors—the nicotinic and the muscarinic acetylcholine receptors. Muscarinic acetylcholine receptors (mAChRs) belong to the superfamily of G-protein coupled receptors that have seven transmembrane domains. There are five subtypes of mAChRs, termed M₁-M₅, and each is the product of a distinct gene. Each of these five subtypes displays unique pharmacological properties. Muscarinic acetylcholine receptors are widely distributed in vertebrate organs,, and these receptors can mediate both inhibitory and excitatory actions. For example, in smooth muscle found in the airways, bladder and gastrointestinal tract, M₃ mAChRs mediate contractile responses. For review, please see (1).

Muscarinic acetylcholine receptor dysfunction has been noted in a variety of different pathophysiological states. For instance, in asthma and chronic obstructive pulmonary disease (COPD), inflammatory conditions lead to loss of inhibitory M₂ muscarinic acetylcholine autoreceptor function on parasympathetic nerves supplying the pulmonary smooth muscle, causing increased acetylcholine release following vagal nerve stimulation. This mAChR dysfunction results in airway hyperreactivity mediated by increased stimulation of M₃ mACbRs. Similarly, inflammation of the gastrointestinal tract in inflammatory bowel disease (IBD) results in M₃ mAChR-mediated hypermotility (3). Incontinence due to bladder hypercontractility has also been demonstrated to be mediated through increased stimulation of M₃ mAChRs. Thus the identification of subtytpe-selective mAChR antagonists may be useful as therapeutics in these mAChR-mediated diseases.

Despite the large body of evidence supporting the use of anti-muscarinic receptor therapy for treatment of a variety of disease states, relatively few anti-muscarinic compounds are in use in the clinic. Thus, there remains a need for novel compounds that are capable of causing blockade at M₃ mAChRs. Conditions associated with an increase in stimulation of M₃ mAChRs, such as asthma, COPD, IBD and urinary incontinence would benefit by compounds that are inhibitors of mAChR binding.

### SUMMARY OF THE INVENTION

This invention provides for a method of treating a muscarinic acetylcholine receptor (mAChR) mediated disease, wherein acetylcholine binds to an M₃ mAChR and which method comprises administering an effective amount of a compound of Formula (I) or a pharmaceutically acceptable salt thereof.

This invention also relates to a method of inhibiting the binding of acetylcholine to its receptors in a mammal in need thereof which comprises administering to aforementioned mammal an effective amount of a compound of Formula (I).

The present invention also provides for the novel compounds of Formula (I), and pharmaceutical compositions comprising a compound of Formula (I), and a pharmaceutical carrier or diluent.

Compounds of Formula (I) useful in the present invention are represented by the structure: 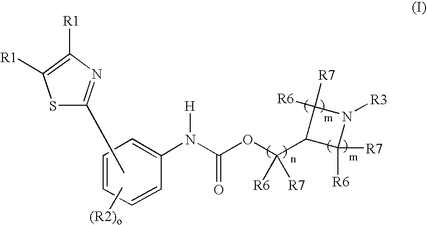
wherein:

R1 is independently selected from the group consisting of hydrogen, halogen, NR₆R₇, OH, ORₐ, C(O)Rₐ, NRₐC(O)ORₐ; OC(O)NR₆R₇; NR₉C(O)Rₐ; C(O)NR₆R₇; C(O)OH; C(O)ORₐ; NHS(O)2Rₐ, C₁₋₅alkyl, aryl, C₁₋₄alkylaryl, C₂₋₄alkenyl; C₂₋₄Aalkenylaryl; cycloalkyl, C₁₋₅ alklcycloalkyl, heteroaryl, C₁₋₄alkylheteroaryl, C₂₋₄ alkenylheteroaryl, heterocyclic, C₁₋₄alkyl heterocyclic, and a C₂₋₄alkenyl moiety heterocyclic, which, when feasible, may be optionally substituted independently by a substituent selected from the group consisting of halogen, nitro, C₁₋₅alkyl, amino, mono or di-C₁₋₄ alkyl substituted amine, ORₐ, C(O)Rₐ, NRₐC(O)ORₐ, OC(O)NR₆R₇, hydroxy, NR₉C(O)Rₐ, S(O)_{m′}Rₐ, C(O)NR₆R₇, C(O)OH, C(O)ORₐ, S(O)₂NR₆R₇, and NHS(O)₂Rₐ; or two R1 moieties together may form a 5 to 6 membered saturated or unsaturated ring; and wherein the alkyl, aryl, arylalkyl, heteroaryl, heteroaryl alkyl, heterocyclic, heterocyclicalkyl groups may be optionally substituted;

R2 is selected from the group consisting of hydrogen, halogen, nitro, cyano, C₁₋₁₀ alkyl, C₂₋₁₀ alkenyl, C₁₋₁₀ alkoxy, halosubstituted C₁₋₁₀ alkoxy; azide, (CR₈R₈)_{q}S(O)ₜRₐ, (CR₈R₈)_{q}ORₐ, hydroxy, hydroxy substituted C₁₋₄alkyl, aryl, aryl C₁₋₄alkyl, aryloxy; arylC₁₋₄ alkyloxy, aryl C₂₋₁₀ alkenyl, heteroaryl, heteroarylalkyl, heteroaryl C₁₋₄ alkyloxy, heteroaryl C₂₋₁₀ alkenyl, heterocyclic, heterocyclic C₁₋₄alkyl, heterocyclicC₂₋₁₀ alkenyl, (CR₈R₈)_{q}NR₄R₅, C₂₋₁₀ alkenyl C(O)NR₄R₅, (CR₈R₈)_{q}C(O)NR₄R₅, (CR₈R₈)_{q} C(O)NR₄R₁₀, S(O)₃R₈, (CR₈R₈)_{q}C(O)R₁₁, C₂₋₁₀ alkenylC(O)R₁₁, (CR₈R₈)_{q}C(O)OR₁₁, C₂₋₁₀alkenylC(O)OR₁₁, (CR₈R₈)_{q}OC(O)R₁₁, (CR₈R₈)_{q}NR₄C(O)R₁₁, (CR₈R₈)_{q} NHS(O)₂R₁₃, (CR₈R₈)_{q} S(O)₂NR₄R₅, (CR₈R₈)_{q}C(NR₄)NR₄R₅, and (CR₈R₈)_{q} NR₄C(NR₅)R₁₁; or two R2 moieties together may form a 5 to 6 membered saturated or unsaturated ring; and wherein the alkyl, aryl, arylalkyl, heteroaryl, heteroaryl alkyl, heterocyclic, heterocyclicalkyl groups may be optionally substituted;

R3 is independently selected from the group consisting of hydrogen, C₁₋₅alkyl, aryl, C₁₋₄alkylaryl, C₂₋₄alkenyl, C₂₋₄alkenylaryl, C₁₋₅ alkylcycloalkyl, cycloalkyl, cycloalkyl C₁₋₅ alkyl, heteroaryl, heteroarylC₁₋₄alkyl, heteroaryl C₂₋₄ alkenyl, heterocyclic, heterocyclic C₁₋₄alkyl, and a heterocyclic C₂₋₄alkenyl moiety, which may be optionally substituted independently by halogen, nitro; halosubstituted C₁₋₄ alkyl, C₁₋₄ alkyl, amino, mono or di-C₁₋₄ alkyl substituted amine, ORₐ, C(O)Rₐ, NRₐC(O)ORₐ, OC(O)NR₆R₇, hydroxy; NR₉C(O)Rₐ, S(O)_{m′}Rₐ, C(O)NR₆R₇, C(O)OH, C(O)ORₐ, S(O)₂NR₆R₇, and NHS(O)₂Rₐ;

R₄ and R₅ are independently selected from the group consisting of hydrogen, optionally substituted C₁₋₄ alkyl, optionally substituted aryl, optionally substituted aryl C₁₋₄alkyl, optionally substituted heteroaryl, optionally substituted heteroaryl C₁₋₄alkyl, heterocyclic, and heterocyclicC₁₋₄ alkyl; or R₄ and R₅ together with the nitrogen to which they are attached form a 5 to 7 member ring which may optionally comprise an additional heteroatom selected from O, N and S;

R₆ and R₇ are independently selected from the group consisting of hydrogen, C₁₋₄ allyl, heteroaryl, aryl, cycloalkyl, and alkyl C₁₋₄ heteroalkyl; or R₆ and R₇ together form a 5 to 7 member ring which ring may optionally contain an additional heteroatom is selected from oxygen, nitrogen or sulfur, and which ring may be optionally substituted;

R₈ is hydrogen or C₁₋₄ alkyl;

R₉ is hydrogen or a C₁₋₄ alkyl;

R₁₀ is C₁₋₁₀ alkyl C(O)₂R₈;

R₁₁ is selected from the group consisting of hydrogen, optionally substituted C₁₋₄ alkyl, optionally substituted aryl, optionally substituted aryl C₁₋₄alkyl, optionally substituted heteroaryl, optionally substituted heteroarylC₁₋₄alkyl, optionally substituted heterocyclic, and optionally substituted heterocyclicC₁₋₄alkyl;

Rₐ is selected from the group consisting of alkyl, aryl, arylC₁₋₄alkyl, heteroaryl, heteroaryl C₁₋₄allyl, heterocyclic, COORₐ, and a heterocyclic C₁₋₄alkyl moiety, all of which moieties may be optionally substituted;

n is an integer having a value of O to 5;

m is an integer having a value of O to 5;

o is an integer having a value of 1 to 4;

q is 0, or an integer having a value of 1 to 10;

s is an integer having a value of 1 to 3;

t is 0, or an integer having a value of 1 or 2; and

m′ is 0, or an integer having a value of 1 or 2.

### DETAILED DESCRITON OF THE INVENTON

This invention relates to novel thiazole aniline compounds, pharmaceutical compositions, processes for their preparation, and use thereof in treating M₃ muscarinic acetylcholine receptor mediated diseases.

In a preferred embodiment of the present invention, the compound is of formula (I) hereinbelow: 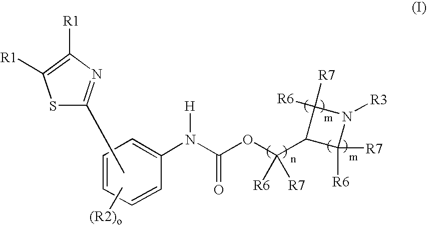
wherein:

the thiazole is ortho to the nitrogen;

R1 is selected from the group consisting of halogen, C₁₋₅alkyl, CH₂F, CHF₂;

R2 is selected from the group consisting of hydrogen, C₁₋₅alkyl, aryl, halogen, hydroxy and alkoxy;

R3 is selected from the group consisting of hydrogen, C₁₋₅alkyl, cycloalkyl, cycloalkyl C₁₋₅ alkyl, C₂₋₄alkenyl, C₂₋₄alkenylaryl; cycloalkyl C₁₋₅ alkyl, and C₁₋₄alkylaryl, which may be optionally substituted independendy by a substituent selected from the group consisting of halogen, nitro, halosubstituted C₁₋₄ alkyl, C₁₋₄ alkyl, amino, mono or di-C₁₋₄ alkyl substituted amine, ORₐ; C(O)Rₐ, NRₐC(O)ORₐ, OC(O)NR₆R₇, hydroxy, NR₉C(O)Rₐ, S(O)_{m′}Rₐ, C(O)NR₆R₇, C(O)OH, C(O)ORₐ, S(O)₂NR₆R₇, and NHS(O)₂Rₐ;

R₆ and R₇ are selected from the group consisting of hydrogen, and C₁₋₄ alkyl, or R₆ and R₇ together form a 5 to 7 member ring which ring may optionally contain an additional heteroatom selected from oxygen, nitrogen or sulfit, and which zing may be optionally substituted;

n is 1 or 2; and independently

m is 1 or 2.

Suitably, R1 is independently selected from the group consisting of hydrogen, halogen, NR₆R₇, OH, ORₐ, C(O)Rₐ, NRₐC(O)ORₐ, OC(O)NR₆R₇, NR₉C(O)Rₐ, S(O)_{m′}Rₐ, C(O)NR₆R₇, C(O)OH, C(O)ORₐ, S(O)₂NR₇, NHS(0)2Rₐ, C₁₋₅alkyl, aryl, C₁₋₄alkylaryl, C₂₋₄Aakenyl, C24alkenylaryl, cycloalkyl, C₁₋₅ alkylcyclalkyl, heteroaryl, C₁₋₄alkylbeteroaryl, C₂₋₄ alkenylheteroaryl, heterocyclic, C₁₋₄alkyl heterocyclic, and a C₂₋₄alkenyl moiety heterocyclic, which when feasible may be optionally substituted independently by a substituent selected fom the group consisting of halogen, nitro, C₁₋₅alkyl, amino, mono or di-C₁₋₄ alkyl substituted amine, ORₐ, C(O)Rₐ, NRₐC(O)ORₐ, OC(O)NR₆R₇, hydroxy, NR₉C(O)Rₐ, S(O)_{m′}Rₐ, C(O)NR₆R₇, C(O)OH, C(O)ORₐ, S(O)₂NR₆R₇, and NHS(O)₂Rₐ. or two R1 moieties together may form a 5 to 6 membered saturated or unsaturated ring; and wherein the alkyl, aryl, arylalkyl, heteroaryl, heteroaryl alkyl, heterocyclic, heterocyclicalkyl groups may be optionally substituted.

Suitably, R2 is selected from the group consisting of hydrogen, halogen, nitro, cyano, C₁₋₁₀ alkyl, C₂₋₁₀ alkenyl, C₁₋₁₀ alkoxy, halosubstituted C₁₋₁₀ alkoxy, azide, (CR₈R₈)_{q}S(O)ₜRₐ, (CR₈R₈)_{q}ORₐ, hydroxy, hydroxy substituted C₁₋₄alkyl, aryl, aryl C₁₋₄ alkyl, aryloxy, arylC₁₋₄ alkyloxy, aryl C₂₋₁₀ alkenyl, heteroaryl, heteroarylalkyl, heteroaryl C₁₋₄ alkyloxy, heteroaryl C₂₋₁₀ alkenyl, heterocyclic, heterocyclic C₁₋₄alkyl, heterocyclicC₂₋₁₀ alkenyl, (CR₈R₈)_{q}NR₄R₅, C₂₋₁₀ alkenyl C(O)NR₄R₅, (CR₈R₈)_{q}C(O)NR₄R₅, (CR₈R₈)_{q} C(O)NR₄R₁₀, S(O)₃R₈, (CR₈R₈)_{q}C(O)R₁₁, C₂₋₁₀ alkenylC(O)R₁₁, (CR₈R₈)_{q}C(O)OR₁₁, C₂₋₁₀alkenylC(O)OR₁₁, (CR₈R₈)_{q}OC(O)R₁₁, (CR₈R₈)_{q}NR₄C(O)R₁₁, (CR₈R₈)_{q} NHS(O)₂R₁₃, (CR₈R₈)_{q} S(O)₂NR₄R₅, (CR₈R₈)_{q}C(NR₄)NR₄R₅, and (CR₈R₈)_{q} NR₄C(NR₅)R₁₁; or two R2 moieties together may form a 5 to 6 membere saturated or unsaturated ring; and wherein the alkyl, aryl, arylalkyl, heteroaryl, heteroaryl alkyl, heterocyclic, hetercyclicallyl groups may be optionally substituted.

Suitably R3 is independently selected from the group consisting of hydrogen, C₁₋₅alkyl, aryl, C₁₋₄alkylaryl, C₂₋₄alkenyl, C₂₋₄alkenylaryl, C₁₋₅ alkylcycloalkyl, cycloalkyl, cycloalkyl C₁₋₅ alkyl, heteroaryl, heteroaryl C₁₋₄alkyl, heteroaryl C₂₋₄ alkenyl, heterocyclic, heterocyclic C₁₋₄alkyl, and a heterocyclic C₂₋₄alkenyl moiety, which may be optionally substituted independently by a substituent selected from the group consisting of halogen, nitro, halosubstituted C₁₋₄ alkyl, C₁₋₄ alkyl, aniino, mono or di-C₁₋₄ akyl substituted amine, ORₐ, C(O)Rₐ, NRₐC(O)ORₐ, OC(O)NR₆R₇, hydroxy, NR₉C(O)Rₐ, S(O)_{m′}Rₐ, C(O)NR₆R₇, C(O)OH, C(O)ORₐ, S(O)₂NR₆R₇, and NHS(O)₂Rₐ,

Suitably, R₄ and R₅ are independently selected from the group consisting of hydrogen, optionally substituted C₁₋₄ alkyl, optionally substituted aryl, optionally substituted aryl C₁₋₄alkyl, optionally substituted heteroaryl, optionally substituted heteroaryl C₁₋₄alkyl, heterocyclic, and heterocyclicC₁₋₄ alkyl, or R₄ and R₅ together with the nitrogen to which they are attached form a 5 to 7 member ring which may optionally comprise an additional heteroatom selected from O, N and S.

Suitably, R₆ and R₇ are independently selected from the group consisting of hydrogen, C₁₋₄ alkyl, heteroaryl, aryl, cycloalkyl, and alkyl C₁₋₄ heteroalkyl; or R₆ and R₇ together form a 5 to 7 member ring which ring may optionally contain an additional heteroatom is selected from oxygen, nitrogen or sulfir, and which ring may be optionally substitued;

Suitably, R₈ is hydrogen or C₁₋₄ alkyl.

Sutiably, R₉ is hydrogen or a C₁₋₄ alkyl.

Sutiably, R₁₀ is C₁₋₁₀ allyl C(O)₂R₈.

Suitably, R₁₁ is selected from the group consisting of hydrogen, optionally substituted C₁₋₄ alkyl, optionally substituted aryl, optionally substituted aryl C₁₋₄alkyl, optionally substituted heteroaryl, optionally substituted heteroarylC₁₋₄alkyl, optionally substituted heterocyclic, and optionally substituted heterocyclicC₁₋₄alkyl.

Suitably, Rₐ is selected from the group consisting of alkyl, aryl, arylC₁₋₄alkyl, heteroaryl, heteroaryl C₁₋₄alkyl, heterocyclic, COORₐ, and a heterocyclic C₁₋₄alkyl moiety, all of which moieties may be optionally substituted.

Suitably, n is an integer having a value of 0 to 5; m is an integer having a value of 0 to 5; o is an integer having a value of 1 to 4; q is 0, or an integer having a value of 1 to 10; s is an integer having a value of 1 to 3; t is 0, or an integer having a value of 1 or 2; m′ is 0, or an integer having a value of 1 or 2.

All of the aryl, heteroaryl, and heterocyclic containing moieties may be optionally substituted as defined herein below.

For use herein the term “the aryl, heteroaryl, and heterocyclic containing moieties” refers to both the ring and the alkyl, or if included, the alkenyl rings, such as aryl, arylalkyl, and aryl alkenyl rings. The term “moieties” and “rings” may be interchangeably used throughout.

As used herein, “optionally substituted” unless specifically defined shall mean such groups as halogen, such as fluorine, chlorine, bromine or iodine; hydroxy; hydroxy substituted C₁₋₁₀alkyl; C₁₋₁₀ alkoxy, such as methoxy or ethoxy; S(O)_{m′}C₁₋₁₀ alkyl, wherein m′ is 0, 1 or 2, such as methyl thio, methyl sulfinyl or methyl sulfonyl; amino, mono & di-substituted amino, such as in the NR₄R₅ group; NHC(O)R₄; C(O)NR₄R₅; C(O)OH; S(O)₂NR₄R₅; NHS(O)₂R₂₀, C₁₋₁₀ alkyl, such as methyl, ethyl, propyl, isopropyl, or t-butyl; halosubstituted C₁₋₁₀ alkyl, such CF₃; an optionally substituted aryl, such as phenyl, or an optionally substituted arylalkyl, such as benzyl or phenethyl, optionally substituted heterocylic, optionally substituted heterocyclicalkyl, optionally substituted heteroaryl, optionally substituted heteroaryl alkyl, wherein these aryl, heteroaryl, or heterocyclic moieties may be substituted one to two times by halogen; hydroxy; hydroxy substituted alkyl; C₁₋₁₀ alkoxy; S(O)_{m′}C₁₋₁₀ alkyl; amino, mono & di-substituted alkyl amino, such as in the NR₄R₅ group; C₁₋₁₀ alkyl, or halosubstituted C₁₋₁₀ alkyl, such as CF₃.

Suitable pharmaceutically acceptable salts are well known to those skilled in the art and include basic salts of inorganic and organic acids, such as hydrochloric acid, hydrobromic acid, sulphuric acid, phosphoric acid, methane sulphonic acid, ethane sulphonic acid, acetic acid, malic acid, tartaric acid, citric acid, lactic acid, oxalic acid, succinic acid, fumaric acid, maleic acid, benzoic acid, salicylic acid, phenylacetic acid and mandelic acid. In addition, pharmaceutically acceptable salts of compounds of Formula (I) may also be formed with a pharmaceutically acceptable cation. Suitable pharmaceutically acceptable cations are well known to those skilled in the art and include alkaline, alkaline earth, ammonium and quaternary ammonium cations.

The following terms, as used herein, refer to:
“halo”—all halogens, that is chloro, fluoro, bromo and iodo.
“C₁₋₁₀alkyl” or “alkyl”—both straight and branched chain moieties of 1 to 10 carbon atoms, unless the chain length is otherwise limited, including, but not limited to, methyl, ethyl, n-propyl, iso-propyl, n-butyl, sec-butyl, iso-butyl, tert-butyl, n-pentyl and the like.
“cycloalkyl” is used herein to mean cyclic moiety, preferably of 3 to 8 carbons, including but not limited to cyclopropyl, cyclopentyl, cyclohexyl, and the like.
“alkenyl” is used herein at all occurrences to mean straight or branched chain moiety of 2-10 carbon atoms, unless the chain length is limited thereto, including, but not limited to ethenyl, 1-propenyl, 2-propenyl, 2-methyl-1-propenyl, 1-butenyl, 2-butenyl and the like.
“aryl”—phenyl and naphthyl;
“heteroaryl” (on its own or in any combination, such as “heteroaryloxy”, or “heteroaryl alkyl”)—a 5-10 membered aromatic ring system in which one or more rings contain one or more heteroatoms selected from the group consisting of N, O or S, such as, but not limited, to pyrrole, pyrazole, furan, thiophene, quinoline, isoqumoline, quinazolinyl, pyridine, pyrimidine, oxazole, tetrazole, thiazole, thiadiazole, triazole, imidazole, or benzimidazole.
“heterocyclic” (on its own or in any combination, such as “heterocyclicalkyl”)—a saturated or partially unsaturated 4-10 membered ring system in which one or more rings contain one or more heteroatoms selected from the group consisting of N, O, or S; such as, but not limited to, pyrrolidine, piperidine, piperae, morpholine, tetrahydropyran, thiomorpholine, or imidazolidine. Furthermore, sulfr may be optionally oxidized to the sulfone or the sulfoxide.
“arylalkyl” or “heteroarylalkyl” or “heterocyclicalkyl” is used herein to mean C₁₋₁₀ alkyl, as defined above, attached to an aryl, heteroaryl or heterocyclic moiety, as also defined herein, unless otherwise indicated.
“sulfinyl”—the oxide S (O) of the corresponding sulfide, the term “thio” refers to the sulfide, and the term “sulfonyl” refers to the fuilly oxidized S(O)₂ moiety.
“wherein two R₁ moieties (or two Y moieties) may together form a 5 or 6 membered saturated or unsaturated ring” is used herein to mean the formation of an aromatic ring system, such as naphthalene, or is a phenyl moiety having attached a 6 membered partially saturated or unsaturated ring such as a C₆ cycloalkenyl, i.e. hexene, or a C₅ cycloalkenyl moiety, such as cyclopentene.

Illustrative compounds of Formula (I) include:
[2-(4-Methyl-thiazol-2-yl)-phenyl]-carbamic acid piperidin-4-ylmethyl ester
[2-(4-Ethyl-thiazol-2-yl)-phenyl]-carbamic acid piperidin-4-ylmethyl ester
{2-[4-(1,1-Difluoro-methyl)-thiazol-2-yl]-phenyl}-carbamic acid piperidin-4-ylmethyl ester
(2-Thiazol-2-yl-phenyl)-carbamic acid piperidin-4-ylmethyl ester; compound with 2,2,2-trifluoro-acetic acid
[2-(4-Propyl-thiazol-2-yl)-phenyl]-carbamic acid piperidin-4-ylmethyl ester
[2-(4-Methyl-thiazol-2-yl)-phenyl]-carbamic acid (2R,6R)-2,6-dimethyl-piperidin-4-ylmethyl ester
[2-(4-Isopropyl-thiazol-2-yl)-phenyl]-carbamic acid piperidin-4-ylmethyl ester
[2-(4-tert-Butyl-thiazol-2-yl)-phenyl]-carbamic acid piperidin-4-ylmethyl ester
[2-(4-Bromo-thiazol-2-yl)-phenyl]-carbamic acid piperidin-4-ylmethyl ester
[2-(4-Chloro-thiazol-2-yl)-phenyl]-carbamic acid piperidin-4-ylmethyl ester
[2-(4-Isobutyl-thiazol-2-yl)-phenyl]carbamic acid piperidin-4-ylmethyl ester
[2-(4-Cyclopropylmethyl-thiazol-2-yl)phenyl]-carbamic acid piperidin-4-ylmethyl ester
[2-(4-Cyclopropyl-thiazol-2-yl)-phenyl]-carbamic acid piperidin-4-ylmethyl ester
[2-(4-Cyclobutyl-thiazol-2-yl)-phenyl]-carbamic acid piperidin-4-ylmethyl ester
[2-(4-Trifluoromethyl-thiazol-2-yl)-phenyl]-carbamic acid piperidin-4-ylmethyl ester
[2-(4-Fluoromethyl-thiazol-2-yl)phenyl]-carbamc acid piperidin-4-ylmethyl ester
{2-[4-(1,1-Difluoro-ethyl)-thiazol-2-yl]-phenyl}-carbanic acid piperidin-4-ylmethyl ester
{2-[4-(2-Fluoro-ethyl)-thiazol-2-yl]-phenyl}-carbamic acid piperidin-4-ylmethyl ester
{2-[4-(2,2-Difluoro-ethyl)-thiazol-2-yl]-phenyl}-carbanic acid piperidin-4-ylmethyl ester
[2-(4-Methoxymethyl-thiazol-2-yl)-phenyl]-carbamic acid piperidin-4-ylmethyl ester
[2-(4-Hydroxymethyl-thiazol-2-yl)-phenyl]-carbamic acid piperidin-4-ylmethyl ester
{2-[4-(1-Hydroxy-ethyl)-thiazol-2-yl]-phenyl}-carbamic acid piperidin-4-ylmethyl ester
{2-[4((R)-1-Hydroxy-ethyl)-thiazol-2-yl]-phenyl}-carbamic acid piperidin-4-ylmethyl ester
{2-[4-(2-Hydroxy-ethyl)-thiazol-2-yl]-phenyl}-carbamic acid piperidin-4-ylmethyl ester
[2-(4-Amino-thiazol-2-yl)-phenyl]-carbamic acid piperidin-4-ylmethyl ester
[5-Fluoro-2-(4methyl-thiazol-2-yl)-phenyl]-carbamic acid piperidin-4-ylmethyl ester
[2-(4-Ethyl-thiazol-2-yl)-4-hydroxy-phenylJarbamic acid piperidin-4-ylmethyl ester
[2-(4-Methyl-thiazol-2-yl)-phenyl]-carbamic acid (2R,6S)-2,6-dimethyl-piperidin-4-ylmethyl ester
[2-(4-Methyl-thiazol-2-yl)-phenyl]-carbamic acid (2R,6S)-2,6-dimethyl-piperidin-4-ylmethyl ester
[2-(4-Methyl-thiazol-2-yl)-phenyl]-carbamic acid (2R,6S)-1-benzyl-2,6-dimethyl-piperidin-4-ylmethyl ester
[2-(4-Methyl-thiazol-2-yl)-phenyl]-carbamic acid (2R,6S)-1-benzyl-2,6-dimethyl-piperidin-4-ylmethyl ester
[2-(4-Methyl-thiazol-2-yl)-phenyl]-carbamic acid (2R,6R)-2,6-dimethyl-piperidin-4-ylmethyl ester
[2-(4-Methyl-thiazol-2-yl)-phenyl]-carbamic acid (2R,6R)-2,6-dimethyl-piperidin-4-ylmethyl ester
[2-(4-Methyl-thiazol-2-yl)-phenyl]-carbamic acid (2R,6R)-1-benzyl-2,6-dimethyl-piperidin-4-ylmethyl ester
[2-(4-Methyl-thiazol-2-yl)-phenyl]-carbamic acid 4-fluoro-piperidin-4-ylmethyl ester
[2-(4-Methyl-thiazol-2-yl)-phenyl]-carbamic acid 1-butyl-piperidin-4-ylmethyl ester
[2-(4-Methyl-5-methylcarbamoyl-thiazol-2-yl)-phenyl]-carbarnic acid piperidin-4-ylmethyl ester
[2-(5-Methyl-thiazol-2-yl)-phenyl]-carbamic acid piperidin-4-ylmethyl ester
[2-(4,5-Dimethyl-thiazol-2-yl)-phenyl]-carbamic acid piperidin-4-ylmethyl ester
[2-(4-Acetyl-thiazol-2-yl)-phenyl]-carbamic acid piperidin-4-ylmethyl ester
{2-[4-(2-Benzyloxy-ethyl)-thiazol -2-yl]-phenyl}-carbamic acid piperidin-4-ylmethyl ester
[2-(4-Methylcarbamoyl-thiazol-2-yl)-phenyl]-carbamic acid piperidin-4-ylmethyl ester
2-[2-(Piperidin-4-ylmethoxycarbonylamino)-phenyl]-thiazole-4-carboxylic acid ethyl ester
[2-(4-Dimethylaminomethyl-thiazol-2-yl)-phenyl]-carbanic acid piperidin-4-ylmethyl ester
[2-(4-Phenyl-thiazol-2-yl)-phenyl]-carbamic acid piperidin-4-ylmethyl ester
[2-(4-Thiophen-3-yl-thiazol-2-yl)-phenyl]-carbanic acid piperidin-4-ylmethyl ester
[2-(4-Ethyl-thiazol-2-yl)4fluoro-phenyl]-carbamic acid piperidin-4-ylmethyl ester
tert-Butyl 4-{[({[4-(4,4,5,5-tetrametyl-[1,3,2]dioxaborolan-2-yl) phenyy]amino}carbonyl)oxy]methyl}piperidine-1-carboxylate
tert-Butyl 4-{[({[3-(4,4,5,5-tetrametyl-[1,3,2]dioxaborolan-2-yl) phenyl]amino}carbonyl)oxy]methyl}piperidine-1-carboxylate
tert-Butyl 4-{[({[4-(4-chloro-1,3-thiazol-2-yl) phenyl]amino}carbonyl)oxy]methyl}piperidine-1-carboxylate
tert-Butyl 4-{[({[3-(4-chloro-1,3-thiazol-2-yl) phenyl]amino}carbonyl)oxy]methyl}piperidine-1-carboxylate
Piperidin-4-ylmethyl 4-(4-chloro-1,3-thiazol-2-yl)phenylcarbamate hydrochloride
Piperidin-4-ylmethyl 3-(4-chloro-1,3-thiazol-2-yl)phenylcarbamate hydrochloride
1-cyclohexylmethyl-piperidin-4-ylmethyl 4-(4-chloro-1,3-thiazol-2-yl)phenylcarbamate
1-cyclohexylmethyl-piperidin-4-ylmethyl 3-(4-chloro-1,3-thiazol-2-yl)phenylcarbamate
4-[4-(4-Chloro-thiazol-2-yl)-phenylcarbamoyloxymethyl]-1-cyclohexylmethyl-1-methyl-piperidinium iodide
4-[3-(4-Chloro-thiazol-2-yl)-phenylcarbamoyloxymethyl]-1-cyclohexylmethyl-1-methyl-piperidinium iodide
4-[4-(4-Chloro-thiazol-2-yl)-phenylcarbamoyloxymethyl]-1,1-dimethyl-piperidinium; and
4-[3-(4-Chloro-thiazol-2-yl)-phenylcarbamoyloxymethyl]-1,1-diethyl-piperidinium; or a pharmaceutically acceptable salt thereof.

Preferred compounds usefuil in the present invention include
[2-(4-Bromo-thiazol-2-yl)-phenyl]-carbamic acid piperidin-4-ylmethyl ester
[2-(4-Chloro-thiazol-2-yl)-phenyl]-carbamic acid piperidin-4-ylmethyl ester
[2-(4-Methyl-thiazol-2-yl)-phenyl]-carbamic acid piperidin-4-ylmethyl ester
{2-[4-(1,1-Difluoro-methyl)-thiazol-2-yl]-phenyl}-carbamic acid piperidin-4-ylmethyl ester
[2-(Fluoromethyl-thiazol-2-yl)-phenyl]-carbamic acid piperidin-4-ylmethyl ester.

Also preferred compounds useful in the present invention include:
[2-(4-Ethyl-thiazol-2-yl)-phenyl]-carbamic acid piperidin-4-ylmethyl ester
(2-Thiazol-2-yl-phenyl)-carbamic acid piperidin-4-ylmethyl ester; compound with 2,2,2-trifluoro-acetic acid
[2-(4-Propyl-thiazol-2-yl)-phenyl]-carbamic acid piperidin-4-ylmethyl ester
[2-(4-Methyl-thiazol-2-yl)-phenyl]-carbamic acid (2R,6R)-2,6-diethyl-piperidin-4-ylmethyl ester
[2-(4-Isopropyl-thiazol-2-yl)-phenyl]-carbamic acid piperidin-4-ylmethyl ester
[2-(4-tert-Butyl-thiazol-2-yl)-phenyl]-carbamic acid piperidin-4-ylmethyl ester
[2-(4-Bromo-thiazol-2-yl)-phenyl]-carbamic acid piperidin-4-ylmethyl ester
[2-(4-Chloro-thiazol-2-yl)-phenyl]-carbamic acid piperidin-4-ylmethyl ester
[2-(4-Isobutyl-thiazol-2-yl)phenyl]-carbamic acid piperidin-4-ylmethyl ester
[2-(4-Cyclopropylmethyl-thiazol-2-yl)-phenyl]-carbamic acid piperidin-4-ylmethyl ester
[2-(4-Cyclopropyl-thiazol-2-yl)-phenyl]-carbamic acid piperidin-4-ylmethyl ester
[2-(4-Cyclobutyl-thiazol-2-yl)-phenyl]-carbamic acid piperidin-4-ylmethyl ester
[2-(4-Trifluorometyl-thiazol-2-yl)-phenyl]carbamic acid piperidin-4-ylmethyl ester
{2-[4-(1,1-Difluoroethyl)thiazol-2-yl]-phenyl}-carbamic acid piperidin-4-ylmethyl ester
{2-[4-(2-Fluoro-ethyl)-thiazol-2-yl]-phenyl}-carbamic acid piperidin-4-ylmethyl ester
{2-[4-(2,2-Difluoroethyl)-thiazol-2-yl]-phenyl}-carbamic acid piperidin-4-ylmethyl ester
[2-(4-Methoxymethyl-thiazol-2-yl)-phenyl]carbarmic acid piperidin-4-ylmethyl ester
[2-(4-Hydroxymethyl-thiazol-2-yl)-phenyl]carbaminc acid piperidin-4-ylmethyl ester
{2-[4-(1-Hydroxy-ethyl)-thiazol-2-yl]-phenyl}-carbamic acid piperidin-4-ylmethyl ester
{2-[4-((R)-1-Hydroxy-ethyl)-thiazol-2-yl]-phenyl}-carbamic acid piperidin-4-ylmethyl ester
{2-[4-(2-Hydroxy-ethyl)-thiazol-2-yl]-phenyl}-carbamic acid piperidin-4-ylmethyl ester
[2-(4-Amino-thiazol-2-yl)-phenyl]arbamnic acid piperidin-4-ylmethyl ester
[5-Fluoro-2-(4-methyl-thiazol-2-yl)-phenyl]-carbamic acid piperidin-4-ylmethyl ester
[2-(4-Ethyl-thiazol-2-yl)-4-hydroxy-phenyl]-carbamic acid piperidin-4-ylmethyl ester
[2-(4-Methyl-thiazol-2-yl)-phenyl]-carbamic acid (2R,6S)-2,6-dimethyl-piperidin-4-ylmethyl ester
[2-(4-Methyl-thiazol-2-yl)-phenyl]-carbamic acid (2R,6S)-2,6-dimethyl-piperidin-4-ylmethyl ester
[2-(4-Methyl-thiazol-2-yl)-phenyl]-carbamic acid (2R,6S)-1-benzyl-2,6-dimethyl-piperidin-4-ylmethyl ester
[2-(4-Methyl-thiazol-2-yl)-phenyl]-carbamic acid (2R,6S)-1-benzyl-2,6-dimethyl-piperidin-4-ylmethyl ester
[2-(4-Methyl-thiazol-2-yl)-phenyl]-carbamic acid (2R,6R)-2,6-dimethyl-piperidin-4-ylmethyl ester
[2-(4-Methyl-thiazol-2-yl)-phenyl]-carbamic acid (2R,6R)-2,6-dimethyl-piperidin-4-ylmethyl ester
[2-(4-Methyl-thiazol-2-yl)-phenyl]-carbamic acid (2R,6R)-1-benzyl-2,6dimethyl-piperidin-4-ylmethyl ester
[2-(4-Methyl-thiazol-2-yl)-phenyl]-carbamic acid 4-fluoro-piperidin-4-ylmethyl ester
[2-(4-Methyl-thiazol-2-yl)-phenyl]arbamic acid 1-butyl-piperidin-4-ylmethyl ester
[2-(4-Methyl-5-methylcarbamoyl-thiazol-2-yl)-phenyl]-carbamic acid piperidin-4-ylmethyl ester
[2-(5-Methyl-thiazol-2-yl)-phenyl]-carbamic acid piperidin-4-ylmethyl ester
[2-(4,5-Dimethyl-thiazol-2-yl)-phenyl]-carbamic acid piperidin-4-ylmethyl ester
[2-(4-Acetyl-thiazol-2-yl)-phenyl]-carbamic acid piperidin-4-ylmethyl ester
{2-[4-(2-Benzyloxy-ethyl)-thiazol-2-yl]-phenyl}-carbamic acid piperidin-4-ylmethyl ester
[2-(4-Methylcarbamoyl-thiazol-2-yl)-phenyl]-carbamic acid piperidin-4-ylmethyl ester
2-[2-(Piperidin-4-ylmethoxycarbonylamino)-phenyl]-thiazole-4-carboxylic acid ethyl ester
[2-(4-Dimethylaminomethyl-thiazol-2-yl)-phenyl]-carbanic acid piperidin-4-ylmethyl ester
[2-(4-Phenyl-thiazol-2-yl)-phenyl]-carbarnic acid piperidin-4-ylmethyl ester
[2-(4-Thiophen-3-yl-thiazol-2-yl)-phenyl]-carbamic acid piperidin-4-ylmethyl ester; and
[2-(4-Ethyl-thiazol-2-yl)-4-fluoro-phenyl]-carbamic acid piperidin-4-ylmethyl ester.

### Methods of Preparation

The compounds of Formula (I) may be obtained by applying synthetic procedures, some of which are illustrated in the Schemes below. The synthesis provided for these Schemes is applicable for producing compounds of Formula (I) having a variety of different R, R1 which are reacted, employing substituents which are suitable protected, to achieve compatibility with the reactions outlined herein. Subsequent deprotection, in those cases, tien affords compounds of the nature generally disclosed. Once the thiazole nucleus has been established, furter compounds of these Formulas may be prepared by applying techniques for functional groups interconversion, well known in the arL While the Schemes are shown with compounds only of Formula (I), this is merely for illustration purpose only.

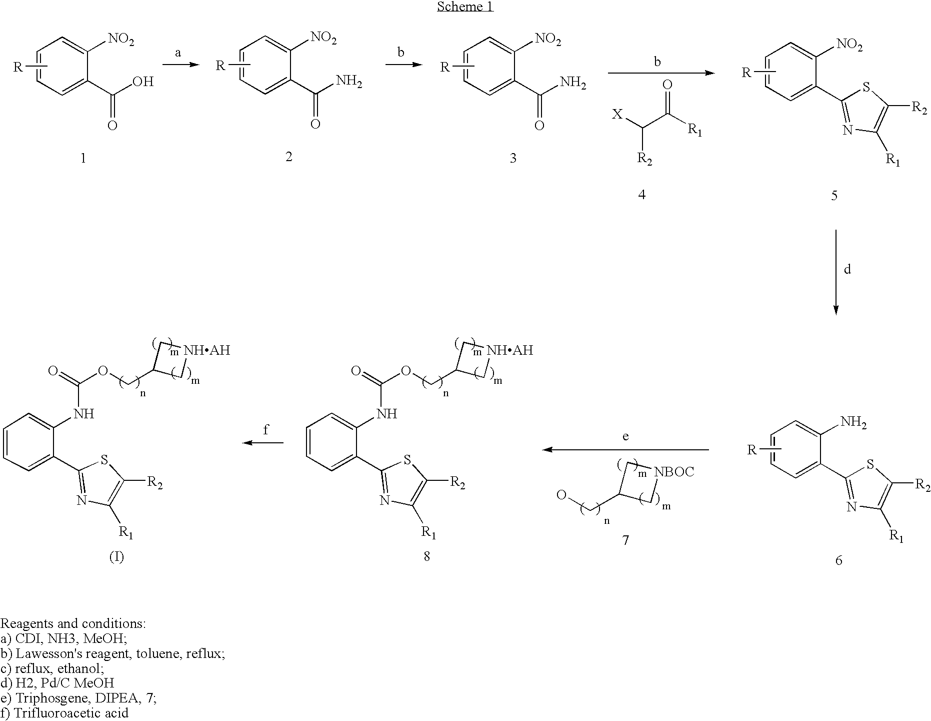

The desired compounds of formula (I) can be prepared as outlined in Scheme 1. The carbamides 2 can be prepared from the corresponding carboxylic acids 1 using standard methods well known in the art such as carbodjimidazole (CDI) in methanolic ammonia. The aryl thioamides 3 can be prepared from the corresponding carbamides 2 using standard reagents well known in the art such as the commercially available Lawesson's reagent. Reacting thioamide 3 with the appropriate ix-halomethylketone 4 in an organic solvent such as ethanol gives the nitro-aryl thiazole 5. The anilines 6 can be prepared from the corresponding nitro-aryl thiazole 5 using standard reduction methods well known in the art such as catalytic hydrogenation. Reacting sequentially the suitably protected aminoalcohol 7 with triphosgene in an organic solvent such as THF, then with the aniline 6 gives the carbamate derivative 8. Removal of the protecting group using standard conditions such as treatment with trifluoroacetic acid in dichloromethane gives the target compound of formula (I).

If the required x-halomethylketone 4 is not commercially available, it can be prepared as outlined in Scheme 2. The commercially available methylketone 9 can be converted to the a-bromomethylketone 4 using standard conditions well known in the art such as bromine in a suitable organic solvent such as methanol.

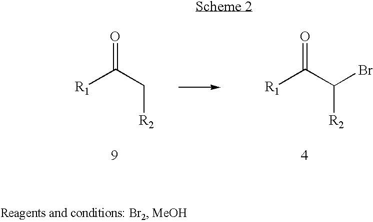

Alternatively, the anilines 6 can be prepared as outlined in Scheme 3. The ortho-substituted carbamide aniline 10 can be converted to the corresponding thioamide 11 by reacting with the Lawesson's reagent at reflux in an organic solvent such as toluene. Reacting the thioamide 11 with the a-halomethylketone 4 in a suitable organic solvent such as ethanol gives the anline-thiazole derivative 6. The thioamide 11 can also be prepared by reacting the ortho-cyanoaniline 12 with gaseous hydrogen sulfide according to known literature procedures (J. Heterocyl. Chem 1974, 11(5), 747-750). The anilines 6 may also be prepared by reacting ortho nitrofluorobenzenes 13 with 2-lithiated thiazole derivatives followed by reduction of the nitro moiety using standard conditions well known in the art such as catlytic hydrogenation in a suitable organic solvent such as ethanol.

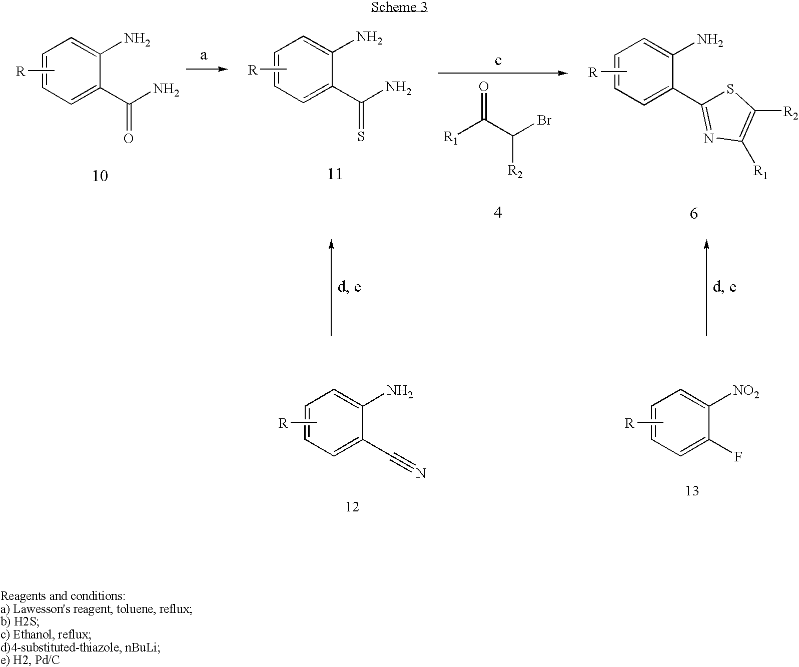

The desired compounds of formula (I) can also be prepared as outlined in Scheme 4. Reacting sequentially the suitably protected aminoalcohol 7 with triphosgene in an organic solvent such as ThF, then with the bromoaniline 14 gives the carbamate derivative 15. Reacting with bis(pinacolato)diboron in the presence of catalytic amounts of palladium (II) chloride following literaure procedure (J. Org. Chem. 2000, 65, 9268-9271) gives the boronate ester 16. The palladium (0) mediated coupling of the boronate ester.16 with the 2-bromothiazole derivative 17 gives the carbamate derivative 8. Removal of the protecting group using standard conditions such as treatment with trifluoroacetic acid in dichloromethane gives the target compound of formula (I).

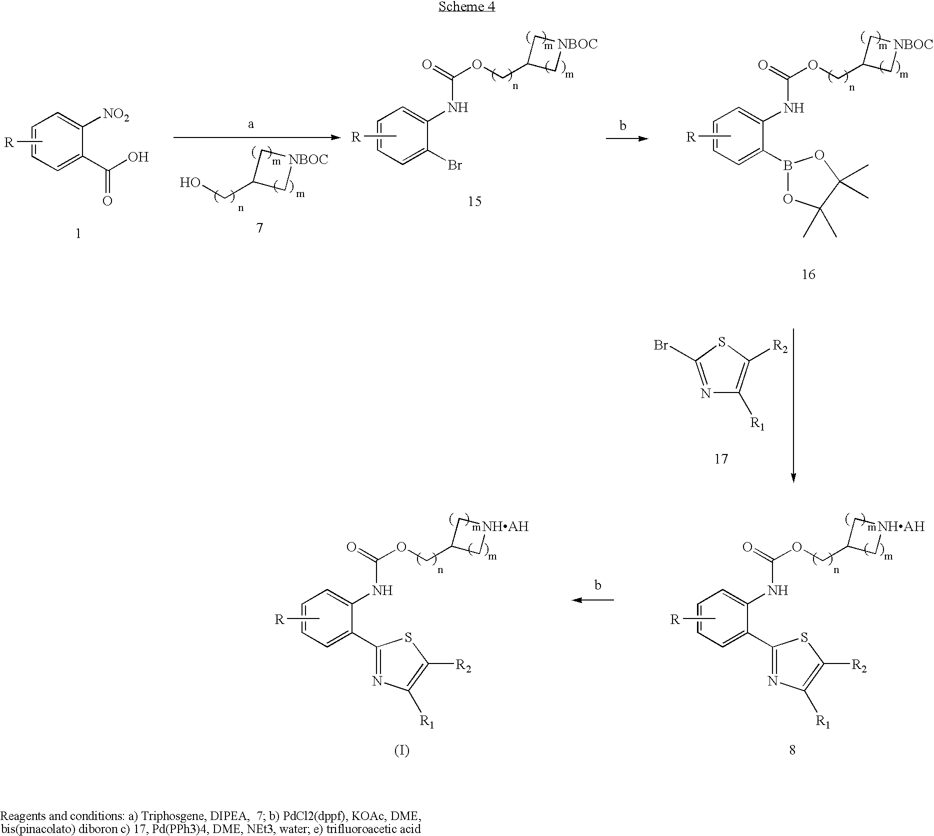

The desired compounds of formula (I) can also be prepared by functionalisation of advance intermediates as outlined in Scheme 5. Reacting the ketone or aldehyde 18 or the alcohol 20 with a fluoxinating agent such as diethylaminosulf ifuoride (DAST) in an organic solvent such as DCM gives the corresponding difluoro derivative 19 or monofluoro derivative 20. Removal of the protecting group on 19 or 20 using standard conditions such as treatment with trfluoroacetic acid in dichloromethane gives the target compounds of formula (I).

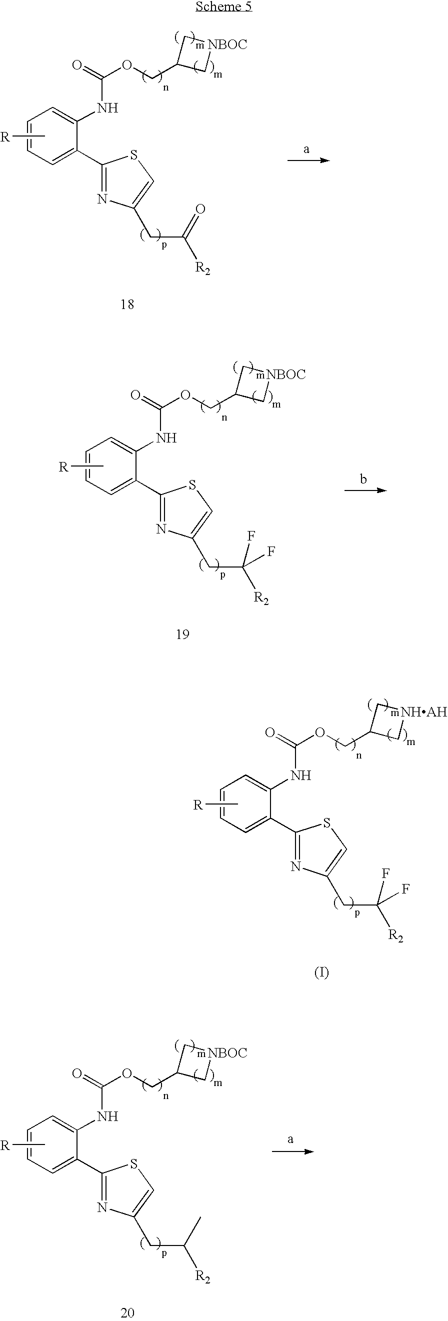

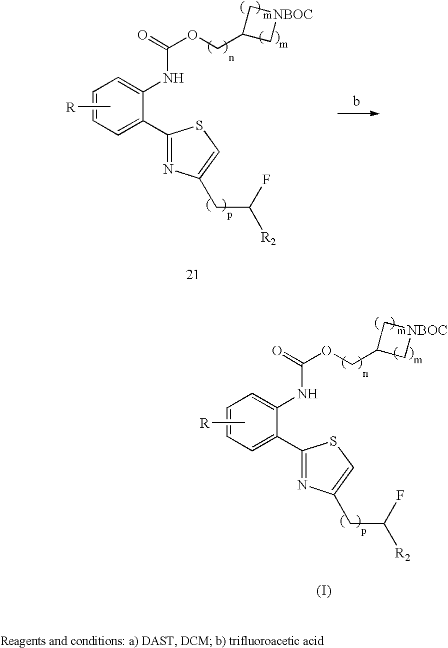

### SYNNTHETIC EXAMPLES

The invention will now be described by reference to the following Examples which are merely illustrative and are not to be construed as a limitation of the scope of the present invention. All temperatures are given in ° C. Thin layer chromatography (t.l.c.) was carried out on silica, and column chromatography on silica (Flash column chromatography using Merck 9385 unless stated otherwise). LC/MS was conducted under the following conditions:
Column: 3.3 cm×4.6 mm ID, 3 um ABZ+PLUS
Flow Rate: 3 ml/min
Injection Volume: 5 μl
Temp: RT
Solvents: A: 0.1% Formic Acid+10 mMolar Ammonium Acetate.

B: 95% Acetonitrile+0.05% Formic Acid

| Gradient: | Time | A % | B % |
|---|---|---|---|
| | 0.00 | 100 | 0 |
| | 0.70 | 100 | 0 |
| | 4.20 | 0 | 100 |
| | 5.30 | 0 | 100 |
| | 5.50 | 100 | 0 |

GC was conducted under the following conditions:
Chemical Ionisation
Instrument HP5973MSD
Column as above
Gradient 80 to 320 at 50 degrees per min.
Gas flow 50 ml min
Run time 10 mins
Chemical ionisation collision gas—Ammonia.(Chemical Ionisation
Instrument HP5973MSD
Column 30 m×0.25 mm HP5
Gradient 80 to 320 at 50 degrees per min.
Gas flow 50 ml min
Run time 10 mins
Chemical ionisation collision gas—Ammonia (except where stated)

¹H-NMR (hereinafter “NMR”) spectra were recorded at 400 MHz using a Bruker DPX 400 spectrometer. Multiplicities indicated are: s=singlet, d=doublet, t-triplet, q=quartet, m=multiplet and br indicates a broad signal. Sat. indicates a saturated solution, eq indicates the proportion of a molar equivalent of reagent relative to the principal reactant.

### Intermediate 1

### 2-Aminobenzenecarbothioamide

Hydrogen sulphide gas (18 g) was bubbled through a solution of 2-aminobenzonitrile (31.48 g) and triethylamine (32 ml) in pyridine (160 ml) for 75 nins. The mixture was stired for 18 h and the solvent evaporated. The residue was trituaed under cyclohexane (300 ml) and filtered to give the title compound as a yellow solid (37.95 g)

NMR (d⁶-DMSO 400 MHz; δ) 9.75 (1H, br s, NH) 9.32 (1H, br s, NH) 7.15 (1H, dd, CH) 7.05 (1H, ddd, CH) 6.70 (1H, dd, CH) 6.52 (1H, ddd, CH) 6.16 (2H, br s, NH₂)

### Inltermediate 2

### 2-(4-methyl-1,3-thiazol-2-yl)aniline

Chloroacetone (1.3 ml) was added to a solution of 2-aminobenzenecarbothioamide (2.0 g) in ethanol (100 ml) and the solution heated under reflux for 18 h. The solvent was evaporated and the residue partitioned between dichloromethane (3×50 ml) and Saturated sodium bicarbonate solution (50 ml). The combined organic extracts were dried (MgSO₄) and the solvent evaporated. The residue was purified by chromatography on silica. Elution with 15% dichloromethane in cyclohexane gave the title compound as a dark red solid (0.86 g)

LC/MS ESI R_{T} 3.37 mins MH⁺ 191.

### 2-(4-methyl-1,3-thiazol-2-yl)aniline (alternative route)

n-Butyl lithium (1.6M in hexanes; 31.5 ml) was added dropwise to a solution of 4-methyl thiazole (5 g) in dry THF (50 ml) at −78° C. under nitrogen over 15 mins. and stirred at −78° C. for 1.5 h. 2-nitrofluorobenzene (7.5 g) in THF (10 ml) was added over 10 mins and the mixture stirred at −78° C. for 0.5 h then allowed to warm to room temperature and stirred for 2 h. The mixture was partitioned between water and ethyl acetate and the organic phase separated, washed with brine and dried (MgSO₄) and evaporated. The crude material was chromatographed on silica Elution with cyclohexane/ethyl acetate 10:1 gave an orange-brown oil. The crude material (1.28 g) in ethanol (60 ml) and water (20 ml) containing HCl in dioxan (4M; 1.75 ml) was hydrogenated over palladium catalyst (10% on carbon; 0.5 g) overnight. The catalyst was filtered off and the solvent evaporated. The residue was partitioned between saturated sodium bicarbonate and ethyl acetate. The organic phase was washed with brine and dried (MgSO₄). The solvent was evaporated to give a brown oil which was purified by chromatography on silica (Merck 7734). Elution with cyclohexane/ethyl acetate 2:1 gave the title compound as a yellow solid (0.54 g)

MS MH⁺ 191 (Thermospray)

### Intermediate 3

### 2-[4-(trifluoromethyl)-1,3-thiazol-2-yl]aniline

3-Bromo-1,1,1-trifluoroacetone (0.98 ml) was added to a solution of 2-aminobenzenecabothioamide (1.2 g) in ethanol (50 ml) and the mixture heated at 70° C. for 22 h. The sovent was evaporated and the residue purified by chromatography on silica. Elution with cyclohexane/dichloromethane 1:1 gave the title compound as a yellow solid (0.98 g)

MS MH⁺ 245 (Thermospray).

NMR (CDCl₃ 400 MHz; δ) 7.63 (1H, s, CH) 7.61 (1H, dd, CH) 7.22 (1H, ddd, CH) 6.77 (1H, dd, CH) 6.72 (1H, ddd, CH) 6.03 (2H, br s, NH₂)

### Intermediate 4

### 2-(4-cyclopropyl-1,3-thiazol-2-yl)aniline

Bromomethyl cyclopropyl ketone (CAS 69276-75-0; 3.09 g) was added to a solution of 2-aminobenzenecarbothioamide (2.2 g) in ethanol (50 ml) and the mixture heated at 70 oc for 22 h. The solvent was evaporated and the residue purified by chromatography on silica. Elution with cyclohexane/dichloromethane 3:1 to methanol (5%) in dichloromethane gave the tide compound as a cream coloured solid (1.17 g)

NMR (CDCl₃ 400 MHz; δ) 7.59 (1H, dd, aromatic CH) 7.14 (1H, ddd, aromatic CH) 6.725 (1H, s, aromatic CH) 6.68 (2H, m, aromatic 2×CH) 6.08 (2H, br s, NH₂) 2.07 (1H, m, CH) 0.94 (4H, m, 2×CH₂)

### Intermediate 5

### 2-(4-phenyl-1,3-thiazol-2-yl)aniline

2-Bromoacetophenone (CAS 70-11-1; 239 mg) was added to a solution of 2-aminophenylthioanmide (152 mg) in ethanol (10 ml). The solution was heated at 80° C. under nitrogen for 6 hr, cooled to room temperature and the solid filtered. The solid was partitioned between sodium bicarbonate (8%) and chloroform. The organic phase was separated and dried over MgSO₄. Evaporation of solvent gave the title compound as lemon solid (128 mg).

LC/MS ESI R_{T} 3.88 mins MH⁺253

### Intermediate 6

### 2-(4-thien-3-yl-1,3-thiazol-2-yl)aniline

1-Bromoaceto-3-thiophene (CAS 1468-82-2; 205 mg) was added to a solution of 2-aminophenylthio˜amide (152 mg) in dimethylfor de (10 ml). The solution was heated at 80° C. under nitrogen for 16 hr. The solvent was evaporated and the residue partitioned between sodium bicarbonate (8%) and dichloromethane. The organic phase was separated and purified by chromatography (Varian Mega Bond Elut®, Si, 5 g). Elution with cyclohexane/dichloromethane (2:1) gave the title compound as a beige solid (100 mg).

LC/MS ESI R_{T} 3.81 mins MH+259

### Intermediate 7

### 2-(4-tert-butyl-1,3-thiazol-2-yl)aniline

1-Bromo-3,3-dimethyl-2-butanone (CAS 5469-26-1; 179 mg) was added to a solution of 2-aminophenyl thioamide (152 mg) in dimethylformamide (10 ml). The solution was heated at 80° C. under nitrogen for 16 hr. The solvent was evaporated and the residue partitioned between sodium bicarbonate (8%) and dichloromethane. The organic phase was separated and purified by chromatography (Varian Mega Bond Elut® Si, 5 g). Elution with cyclohexane/dichloromethane (2:1) gave the title compound as a yellow oil (175 mg).

LC/MS ESI R_{T} 3.86 mins MH⁺233

### Intermediate 8

### 2-(4,5-dimethyl-1,3-thiazol-2-yl)aniline

3-Bromo-2-butanone (CAS 814-75-5; 151 mg) was added to a solution of 2-aminophenylthioamide (152 mg) in dimethylformamide (10 ml). The solution was heated at 80° C. under nitrogen for 16 hr. The solvent was evaporated and the residue partitioned between sodium bicarbonate (8%) and dichloromethane. The organic phase was separated and purified by chromatography (Varian Mega Bond Elut®, Si, 5 g). Elution with cyclohexane/dichloromethane (2:1) gave the title compound as a yellow solid (74 mg).

LC/MS ESI R_{T} 3.59 mins MH⁺=205

### Intermediate 9

### 2-(4-ethyl-1,3-thiazol-2-yl)aniline

1-Bromo-2-butanone (CAS 816400; 180 mg) was added to a solution of 2-aminophenylthioamide (152 mg) in dimethylformamide (10 ml). The solution was heated at 80° C. under nitrogen for 3 hr. The mixture was quenched with 5% diethylamine in ethanol at 50° C. for 2 hr, cooled to room temperature, sodium bicarbonate solution (8%) added and extracted with dichloromethane. The organic phase was separated, diluted with cyclohexane (1:3) and purified by chromatography (Varian Mega Bond Elut®, Si, 5 g). Elution with cyclohexane/dichloromethane (stepped gradient) gave the title compound as a yellow oil (150 mg).

LC/MS ESI R_{T} 3.44 mins (not ionised well)

### Intermediate 10

### 2-(5-methyl-1,3-thiazol-2-yl)aniline

2-Bromo-propanal (CAS 19967-57-8; 165 mg) was added to a solution of 2-aminophenylthioarnide (152 mg) in dry ether (20 ml). Triethylamine (200 ul) was added and the mixture heated at 80° C. under nitrogen for 6 hr. Water was added, and the miixture extracted with dichloromethane. The organic phase was separated, dried over MgSO₄, filtered and evaporated down. The residue was purified by chromatography (Biotage Flash 40i™, silica) and elution with ethyl acetate (1:10 then 3:10) gave the uncyclised material. The residue was dissolved in concentrated HCl (4 ml) and heated at 60° C. for 3 hr, cooled to room temperature and basified with sodium bicarbonate solution (8%). The product was extracted into dichloromethane, dried over MgSO4, filtered and the solvent evaporated. The residue was purified by Varian Mega Bond Elut®, Si, 5 g), elution with cyclohexane/dichloromethane (1:1) gave the title compound as a yellow oil (66 mg).

LC/MS ESI R_{T} 3.37 mins (not ionised well)

MS Thermospray MH⁺=191

### Intermediate 11

### 2-(4isopropyl-1,3-thiazol-2-yl)anline

1-Bromo-3-methyl-2-butanone (CAS 19967-55-6; 164 mg) was added to a solution of 2-aminophenyl thioamide (152 mg) in ethanol (10 ml). The solution was heated at 80° C. under nitrogen for 5 hr. The solvent was evaporated and the residue dissolved in DCM, washed with sodium bicarbonate solution (8%), semi-saturated brine solution, and dried over MgSO₄. The mixture was filtered, the solvent eavaporated and the reisdue purified by chromatography (Varian Mega Bond Elut®, Si, 5 g). Elution with cyclohexane/dichloromethane (1:2) gave the title compound (138 mg).

LC/MS ESI R_{T} 3.70 mins MH⁺219

### Intermediate 12

### 2-(4-propyl-1,3-thiazol-2-yl)aniline

To a solution of 2-aminobenzenecarbothioamide (714 mg) in ethanol (50 ml) was added 1-bromo-pentan-2-one (CAS-Number 817-71-0; 976 mg). The reaction mixture was stirred for 4 h at 80° C. and then 16 h at room temperature. A white suspension had formed. The solvent was evaporated and the residue partitioned between dichloromethane (30 ml) and 2N sodium bicarbonate (40 ml). The aqueous phase extracted with dichloromethane (40 ml×2). The combined organic extracts were washed with water (30 ml) and brine (30 ml) and dried (Na₂SO₄). The solvent was evaporated and the residue purified by (Varian Mega Bond Elut®, Si, 10 g). Elution with 15% dichloromethane/cyclohexane gave the title compound as a yellow oil (626 mg).

LC/MS ESI R_{T} 3.72 mins MH⁺219

Tlc SiO₂ (Cyclohexane/Ethyl acetate 2:1) Rf 0.5

### Intermediate 13

### 2-(4-pentyl-1,3-thiazol-2-yl)aniline (A) and 2-(5-butyl-4-methyl-1,3-thiazol-2-yl) aniline (B)

To a solution of 2-aminobenzenecarbothioamide (1.73 g) in absolute ethanol (60ml) was added 1-bromo-heptan-2-one (CAS No. 16339-93-8;1.9 g; This was contaminated with 40% of 3-bromo-heptan-2-one;CAS No. 51134-59-9)

The reaction mixture was stirred for 3.5 h at 80° C. and then 16 h at room temperature. The solvent was evaporated and the residue partitioned between dichloromethane (40 ml) and 2N sodium bicarbonate (40 ml). The combined organic extracts were washed with water (60 ml) and brine (60 ml) and dried Na₂SO₄). The solvent was evaporated to give a mixture of the title compounds (A) and (B) as a yellow oil (1.4 g)

NMR (CDCl₃ 400 MHz; δ) 7.61 (1H, dd, CH), 7.15 (1H, ddd, CH), 6.78-6.68 (3H, m, 3×CH), 6.10 (2H, br s, NH₂), 2.78 (2H, t, CH₂), 1.8-1.2 (6H, m, 3×CH₂), 0.94 (3H, t, CH₃)

(B) 2.35 (3H, thiazole CH₃)

### Intermediate 14

### 2-(4-butyl-1,3-thiazol-2-yl)aniline

To a solution of 2-aminobenzenecarbothioamide (800 mg) in absolute ethanol (50 ml) was added 1-bromo-hexan-2-one (CAS-Number 26818-07-5; 1.1 g). The reaction mixture was stirred for 4 h at 80° C. and then 5 days at room temperature. The solvent was evaporated and the residue partitioned between dichloromethane (40 ml) and 2N sodium bicarbonate (40 ml). The combined organic extracts were washed with water (50 ml) and brine (50 ml) and dried (Na₂SO₄). The solvent was evaporated and the residue purified by (Varian Mega Bond Elut®, Si, 10 g). Elution with 0%-15% dichloromethane/cyclohexane gave the title compound as a yellow oil (677 mg)

LC/MS ESI R_{T} 3.58 mins MH⁺233

Tlc SiO₂ (Dichloromethane) R_{f} 0.65

### Intermediate 15

### 2-(2-Aminophenyl)-N,4-dimethyl-1,3-thiazole-5-carboxamide

To a solution of 2-aminobenzenecarbothioamide (411 lmg) in absolute ethanol (20 ml) was added 2-chloro-N-methylacetoacetamide (CAS-number 4116-10-3; 550 mg). The resultant yellow solution was stirred at 80° C. for 4 h and then at room temperature for 18 h. A white suspension had formed which was filtered off under vacuum. The filtrate was concentrated in vacuo. The resultant orange oil was partitioned between dichloromethane (40 ml) and 2N sodium bicarbonate (40 ml). The aqueous phase was extracted with dichloromethane (40 ml×2). The combined organics were washed with water (80 ml) and brine (80 ml), dried (Na₂SO₄) and concentrated to leave a yeuow oil. This was purified by Biotage Flash 40i™, silica Elution with 1:1, cyclohexane/ethyl acetate afforded the title compound as a yellow oil (45 mg)

LC/MS ESI R_{T} 2.89 mins MH⁺ 248

Tlc SiO₂ (ethyl acetate/cyclohexane, 1:1) R_{f} 0.5

### Intermediate 16

### 2-{4-[2-(Benzyloxy)ethyl]-1,3-thiazol-2-yl}aniline

A solution of 4-benzyloxy-1-bromo-2-butanone (7.1 g) and 2-aminobenzenecarbothioamide (4 g) in DMF (50 ml) was heated at 80° C. for 3 hours. After cooling to room temperature, the reaction mixture was partitioned between diethyl ether (500 ml) and water (100 ml). The aqueous layer was separated and extracted with diethyl ether (2×150 ml). The organic extracts were combined, dried (MgSO₄) and evaporated to give an oily residue which was purified by flash chromatography on silica. Elution with hexane/ethyl acetate 4:1 gave the title comipound as a colourless oil (2.0 g).

LC/MS ESI R_{T} 3.88 mins MH⁺311

### Intermediate 17

### Ethyl [2-(2-aminophenyl)-1,3-thiazol-4-yl]acetate

A mixture of ethyl 4-bromoacetoacetate (2.7 g) and 2-aminobenzenecarbothioamide (2 g) in DMF (25 ml) was heated at 80° C. for 2 hours then cooled to room temperature. The reaction mixture was then partitioned between diethyl ether (150 ml) and water (200 ml). The aqueous layer was separated, extracted with diethyl ether (150 ml) then basified to pH 8 with 0.5N aqueous sodium hydroxide then freer extrcted with diethyl ether (150 ml). The organic extracts were combined, dred (MgSO₄) and evaporated to give an oily residue which was purified by flash chromatography on silica. Elution with hexanelethyl acetate 4:1 gave the title compound as a colourless oil (1.46 g).

LC/MS ESI R_{T} 3.39 mins MH⁺263.1

### Intermediate 18

### 2-Nitrobenzenecarbothioamide

A mixture of o-nitrobenzaaide (10 g) and 2,4-bis(4-methoxyphenyl)-1,3-dithia-2,4-diphosphetane-2,4-disulfide (15.6 g) in toluene (150 ml) was heated at reflux for 2 hours then allowed to cool to room temperature. Silica gel (Merck 9385) was then added to the reaction mixture and the solvent was evaporated. The resulting residue, pre-absorbed on silica, was purified by flash chromatography. Elution with cyclohexane/ethyl acetate 3:1 afforded the title compound as a yellow solid (7.9 g).

NMR (CDCl₃ 400 MHz; δ) 8.15 (1H, dd, aromatic CH) 7.78 (1H, br s, NH₂) 7.65 (1H, dt, aromatic CH) 7.57-7.51 (2H, m, aromatic CH) 7.09.

### Intermediate 19

### Ethyl [2-(2-nitrophenyl)-1,3-thiazol-4-yl]acetate

A solution of 2-nitrobenzenecarbothioamide (1 g) and ethyl bromoacetoacetate (1.15 g) in DMN (10 ml) was heated at 80° C. for 2 hours then cooled to room temperature. The mixture was then partitioned between water (100 ml) and diethyl ether (200 ml). The aqueous phase was separated and extracted with diethyl ether (100 ml). The combined organic layers were dried (MgSO₄) and evaporated. The residue was purified by flash chromatography. Elution with cyclohexane-ethyl acetate 3/1 gave the tide compound (1.15 g).

LC/MS ESI R_{T} 3.21 mins MH⁺293

### Intermediate 20

### [2-(2-Nitrophenyl)-1,3-thiazol-4-yl]acetaldehyde

To a solution of ethyl [2-(2-nitrophenyl)-1,3-thiazol-4-yl]acetate (150 mg) in dichloromethane (3 ml) at −78° C. was added a 1M solution of diisobutylaluminum hydride in toluene (0.77 ml) over 10 mins. The reaction mixture was stirred at −78° C. for 2 hours then treated with methanol (1 ml) and allowed to warm-up to room temperature. A saturated aqueous solution of potassium sodium tartrate (3 ml) was added and the resulting mixture was stirred at room temperature for 2 hours. The reaction mixture was hen partitioned between diethyl ether (200 ml) and water (150 ml). The aqueous layer as separated and extracted with diethyl ether (50 ml). The combined organic layers ere dried (MgSO₄) and evaporated to afford the title compound (130 mg).

LC/MS ESI R_{T} 2.49 mins MH⁺249.3

### Intermediate 21

### (R)-4-(1-Hydroxylethyl)-2-(2-nitrophenyl)-1,3-thiazole

A 1.6M solution of n-butyl lithium in hexanes (93.6 ml) was added to diisopropylamine (20.5 ml) in THF (30 ml) at −70° C. The resulting cloudy solution was stirred at that temperature for 20 mins. A solution of chloroacetic acid (7.0 g) in THF (70 ml) was then slowly added over 70 mins, the temperature being kept between −60° C. and −70 ° C. throughout the addition. After stirring for a further hour at −70° C., the anionic solution was carefully transferred via cannula to a solution of methyl (R)-lactate in THF (50 ml) at 0° C. The temperature inside the reaction vessel dropped to −10° C. after completion of the addition. The reaction mixture was then cooled to −70 ° C. and stirred at that temperature for 30 mins before being carefully quenched with acetic acid (15 ml). After slowly warming up to room temperature over 16 hours, the white. slurry was partitioned between ethyl acetate (300 ml) and water (200 ml). The aqueous layer was separated and extracted with ethyl acetate (200 ml). The combined organic extracts were washed with saturated aqueous sodium bicarbonate (2×100 ml), dried (MgSO₄) and evaporated to give an oily residue (1.4 g).

The acid chloride thus obtained was added to a solution of 2-nitrobenzamidecarbothioamide (1 g) in DMF (30 ml) and the resulting solution was stired at 80° C. for 2 hours before being cooled to room temperature. The reaction mixture was diluted with ether (300 ml) and washed with water (100 ml). The aqueous was re-extracted with ether (2×150 ml). The combined organic extracts were evaporated to give a crude oil that was then purified by two successive flash column chromatographies. Elution with ethyl acetate/cyclohexane 1:3 and increasing the polarity to neat ethyl acetate, afforded the title compound (583 mg) as a yellow oil.

LCMS R_{T} 2.82 mins (not ionised well)

NMR (d⁶DMSO 400 MHz; δ) 7.96 (1H, d, aromatic CH) 7.88 (1H, d, aromatic CH) 7.79 (1H, t, aromatic CH) 7.72 (1H, t, aroratic CH) 7.59 (1H, s, thiazole CH) 5.42 (1H, d, OH) 4.80 (1H, p, CHOH) 1.36 (3H, d, CH₃)

### Intermediate 22

### 4-(2,2-Difluoroethyl)-2-(2-nitrophenyl)-1,3-thiazole

A solution of [2-(2-nitrophenyl)-1,3-thiazol-4-yl]acetaldehyde (130 mg) and (diethylamino)sulfur trifluoride (0.131 ml) in dichloromethane (1 ml) was stifed at room temperature for 2 hours 20 miis. More (diethylamino)sulfur trifluoride (0.05 ml) was then added and the solution was stirred at room temperature for a further 2 hours. The reaction mixture was then diluted with dichloromethane (100 ml) and washed with saturated aqueous sodiumbicarbonate (50 ml). The aqueous phase was separated, extracted with dichloromethane (50 ml). The combined organic extracts were dried (MgSO₄) and evaporated The resulting crude material was purified by flash chromatography. Elution with ethyl acetate/hexane 1:3 gave the title compound as a colourless oil (72 mg).

LC/MS ESI R_{T} 3.24 mins MH⁺271

### Intermediate 23

### 2-[4-(2,2-Difluoroethyl)-1,3-thiazol-2-yl]aniline

A mixture of 4-(2,2-difluoroethyl)-2-(2-nitrophenyl)-1,3-thiazole (68.5 mg) and 10% palladium hydroxide on carbon (130 mg) in ethanol (3 ml) was treated with hydrogen over 3 hours. The catalyst was filtered off over the filter agent Celite® and the filtrate was evaporated to give the title compound as a yellow oil (46.5 mg).

LC/MS ESI R_{T} 3.40 mins MH⁺241.3

### Intermediate 24

### Ethyl 2-(2-aminophenyl)-1,3-thiazole-4-carboxylate

To a solution of 2-aminobenzenecarbothioamide (1.01 g) in anhydrous DMF (12.5 ml) was added dropwise ethylbromopyruvate (1.10 g). The solution was sti at 80° C. for 1.5 hours. The resultant mixture was cooled and partitioned between ethyl acetate (3×50 ml) and water (50 ml). The combined organics were evaporated and the residue purified by flash chromatography.Elution with.hexane: ethyl acetate (9:1) gave the title compound (657 mg)

NMR (DMSO, 400 MHz; δ) 8.47 (1H, s, aromatic CH) 7.58 (1H, d, aromatic CH) 7.18 (1H, dd, aromatic CH) 7.10 (2H, br s, NH₂) 6.82 (1H, d, aromatic CH) 6.62 (1H, dd, aromatic CH) 4.34 (2H, q, CH₂) 1.33 (3H, t, CH₃)

### Intermediate 25

### 2-(2-Aminohenyl)-1,3-thiazole-4-carboxylic acid

Ethyl 2-(2-aminophenyl)-1,3-thiazole-4-carboxylate (198 mg) was dissolved in ethanol (15 ml) by heating to 50° C. Water (1 ml) and potassium hydroxide (225 mg) were added and the suspension was stirred at 54 ° C. for 3 hours. The mixture was evaporated and partitioned between water (25 ml) and ethyl acetate (25 ml). The aqueous layer was acidified to pH 1 using hydrochloric acid (2N, aqueous). Further ethyl acetate (25 ml) was added to dissolve the precipitate. The layers were separated and the aqueous layer further extracted with ethyl acetate (2×25 ml). The combined organics were washed with brine (25 ml), and then evaporated to dryness. The solid was triturated with ethyl acetate to yield the title compound (245 mg, contaminated with sodium chloride).

NMR (DMSO, 400 MHz, δ) 8.39 (1H, s, aromatic CH) 7.59 (1H, br d, aromatic CH) 7.17 (1H, br t, aromatic CH) 7.12 (2H, br s, NH₂) 6.84 (1H, d, arornatic CH) 6.62 (1H, aromatic CH)

### Intermediate 26

### 2-(2-Aminophenyl)-N-methyl-1,3-thiazole-4-carboxamide

A suspension of 2-(2-anophenyl)-1,3-thiazole-4-carboxylic acid (140 mg), WSCDI (112 μl), hydroxybenzotriazole (90.2 mg) in tetrahydrofuran (2 ml) was stirred at 20° C. under nitrogen for 45 minutes before adding methylamine (2M in tetrahydrofuran, 335 μl). The mixture was stired for 2 hours at 20° C. then diluted with dichloromethane (20 ml). This mixture was washed with hydrochloric acid (2M, 20 ml), sodium bicarbonate (1M, 20 ml) and the organic layer evaporated to yield the title compound (11 mg). The aqueous hydrochloric acid layer was basified to pH 8 using sodium bicarbonate (70 ml) and extracted using dichloromethane (3×20 ml). The combined organics were dried MgSO₄) and evaporated to yield the title compound (60.3 mg).

LC/MS ESI R_{T} 2.89 mins MH⁺234

### Intermediate 27

### 2-(4-Cyclobutyl-1,3-thiazol-2-yl)aniline

A solution of 2-bromo-1-cyclobutylethanone (CAS number 128312-69-6, 354 mg) in absolute alcohol (5 ml) was added dropwise to a solution of 2-aminobenzenecarbothioamide (152 mg) in absolute alcohol (5 ml) and the resulting mixture was heated at 80° C. under nitrogen for 3 h. A few drops of concentrated hydrochloric acid were added and the mixture was heated for a firther 2 h. The solvent was removed and the residue was partitioned between sodium bicarbonate (8%) and ethyl acetate. The combined organic extracts were washed with brine and dried (Na₂SO₄) and the solvent was evaporated. The residue was purified (Varian Mega Bond Elut®) using cyclohexane (×3) and dichloromethane (×3) as eluant. The appropriate fractions were concentrated in vacuo to give the title compound (198 mg).

LC/MS ESI R_{T} 4.0 mins MH⁺ 231

### Intermediate 28

### 2-(Cyclohexyl-1,3-thiazol-2-yl)aniline

A solution of 2-bromo-1-cyclohexylethanone (CAS number 56077-28-2, 354 mg) in absolute alcohol (5 ml) was added dropwise to a solution of 2-aminobenzenecarbothioamide (152 mg) in absolute alcohol (5 ml) and the resulting mixture was heated at 80° C. under nitrogen for 6 h. The solvent was removed and the residue was partitioned between sodium bicarbonate (8%) and ethyl acetate. The combined organic extracts were washed with brine and dried (Na₂SO₄) and the solvent was evaporated. The residue was purified (Varian Mega Bond Elut®, Si) using cyclohexane (×3) and dichloromethane (×3) as eluanl This gave the title compound (167 mg).

LC/MS ESI R_{T} 4.21 mins MH⁺ 259

### Intermediate 29

### 2-(4-Cyclopentyl-1,3-thiazol-2-yl)aniline

A solution of 2-bromo-1-cyclopentylethanone (CAS number 52423-62-8, 191 mg) in absolute alcohol (5 ml) was added to a solution of 2-aminobenzenecarbothioamide (152 mg) in absolute alcohol (5 ml) and the resulting mixture was heated at 80° C. under nitrogen for 6 h. The solvent was removed and the residue was partitioned between sodium bicarbonate (8%) and ethyl acetate. The combined organic extracts were washed with brine and dried (Na₂SO₄) and the solvent was evaporated. The residue was purified (Varian Mega Bond Elut®, Si) using cyclohexane (×3) and dichloromethane (×3) as eluant. This gave the title compound (60 mg).

LC/MS ESI R_{T} 4.05 mins MH⁺ 245

### Intermediate 30

### 1-Bromo-3-cyclopropylacetone

Bromine (0.58 ml) was added in a slow and steady stream to a solution of 1-cyclopropylacetone [may be prepared by literature methods, such as described in Yoshio Ueno et al, Tetrahedron Lett. (1982), 23(25), 2577-80] (1.1052 g) in dry methanol (9 ml) at −10° C. The solution was warmed to 7° C. and stirred for 40 miins, then hydrogen chloride (1M in diethyl ether, 0.25 ml) was added and the mixture stirred for 3.5 h at 5-10° C. To the reaction was added aqueous sodium thiosulphate solution (1M; 6 ml) dropwise until decolourisation occurred followed by water (2 ml). The reaction was extracted into diethyl ether (×2) and the combined organics were washed with saturated aqueous sodium bicarbonate solution and brine, dried over anhydrous rnagnesium sulphate, filtered and evaporated in vacuo to give the title compound as a light yellow oil (1.1 g)

NMR (CDCl₃ 400 MHz; δ) 3.96 (2H, s, CH₂) 2.56 (2H, d, CH₂) 1.03 (1H, m, CH) 0.61 (2H, m, CH₂) 0.18 (2H, m, CH₂)

### Intermediate 31

### 2-[4 Cyclopropylmethyl)-1.3-thiazol-2-yl]aniline

1-Bromo-3-cyclopropylacetone (500 mg) in absolute ethanol (14 ml) was added to a solution of 2-aminobenzenecarbothioamide (430 mg) in absolute ethanol (14 ml). The mixture was stirred for 6.5 h at 80° C. under nitrogen, cooled to room temperature and evaporated in vacuo. The residue was partitioned between ethyl acetate (×2) and saturated aqueous sodium bicarbonate solution. Combined organics were washed with brine, dried over anhydrous magnesium sulphate, filtered and concentrated in vacuo. The oil was purified by Varian Mega Bond Elut® (Si, 10 g); elution with 0-6% dichloromethane in cyclohexane gave the title compound as a yellow oil (248 mg).

LC/MS ESI R_{T} 3.88 mins MH⁺ 231

### Intermediate 32

### 1-Bromo-4-methylpentan-2-one

Bromine (0.77 ml) was added in a slow and steady stream to a solution of 4methyl-2-pentanone (1.5 g) in dry methanol (12 ml) at −10° C. The solution was warmed to 7° C. and stirred for 1 h. To the reaction was added aqueous sodium thiosulphate solution dropwise until decolourisation occurred followed by saturated aqueous sodium bicarbonate solution until neutral. The reaction was extracted into diethyl ether (×2) and the combined organics were washed with brine, dried over anhydrous magnesium sulphate, filtered and evaporated in vacuo to give the title compound as a colourless oil (2.1 g)

NMR (CDCl₃ 400 MHz; δ) 3.88 (2H, s, CH₂Br) 2.54 (2H, d, CH₂) 2.18 (1H, m, CH) 0.93 (6H, d, 2×CH₃)

### Intermediate 33

### 2-(4-Isobutyl-1,3-thiazol-2-yl)aniline

1-bromo-4-methylpentan-2-one (500 mg) in absolute ethanol (14 ml) was added to a solution of 2-aminobenzenecarbothioamide (426 mg) in absolute ethanol (14 ml). The mixture was sti for 5 h at 80° C. under nitrogen, cooled to room temperature and evaporated in vacuo. The residue was partitioned between ethyl acetate (×2) and saturated aqueous sodium bicarbonate solution. Combined organics were washed with brine, dried over anhydrous magnesium sulphate, filtered and evaporated in vacuo. The oil was purified by Varian Mega Bond Elut® (Si, 10 g); elution with cyclohexane followed by 0-3% dichloromethane in cyclohexane gave the title compound as a yellow oil (560 mg).

LC/MS ESI R_{T} 4.02 mins MH⁺ 233

### Intermediate 34

### (R)-2-[4-(1-Hydroxyethyl)-1,3-thiazole-2-yl]aniline

A mixture of (R)-4-(1-hydroxylethyl)-2-(2-nitrophenyl)-1,3-thiazole (100 mg) and 10% palladium hydroxide on carbon (80 mg) in ethanol (4 ml) was treated with hydrogen over 3 hours. The catalyst was filtered off over the filter agent Celite® and the filtrate was evaporated to give the title compound as a pale brown oil (83.3 mg).

LC/MS R_{T} 2.93 mins (not ionised well)

NMR (d⁶DMSO 400 MHz; δ) 7.60 (1H, d, aromatic CH) 7.14 (1H, t, aromatic CH) 7.03 (1H, s, thiazole CH) 6.76-6.67 (2H, m, aromatic 2×CH) 6.04 (2H, br. s, NH₂) 5.01 (1H, q, CHOH) 2.53 (1H, br.s, OH) 1.60 (3H, d, CH₃)

### Intermediate 35

### 4-Fluoro-2-nitrobenzenecarbothioamide

A mixture of 4-fluoro-2-nitrobenzamide (1.42 g), 2,4-bis(4-methoxyphenyl)-1,3,2,4-dithiadiphosphetane 2,4-sulfide (2.11 g), and toluene (30 ml) was heated at reflux under nitrogen for 3 h. The cooled mixture was concentrated and the residue adsorbed from ethyl acetate (35 ml) onto silica gel (Merck 7734, 24 ml). The resultant silica was purified by applied as a solid plug to a Biotage Flash™, silica column (90 g), and this eluted with ethyl acetateyclohexane (gradient 1:19 to 3:7) to give the title compound as orange crystals (869 mg).

LC/MS ESI R_{T} 2.45 mins, MH⁺199.

### Intermediate 36

### 5-Fluoro-2-nitrobenzenecarboamide

A solution of 5-fluoro-2-nitrobenzoic acid (10 g) and 1,1 carbonyldiimidazole (9.5 g) in THF (90 ml) was stirred for 1.5 hours at room temperature. 2M methanolic ammonia (40 ml) was added and the resulting solution was stirred for another 18 hours. The solvents were evaporated, then ethyl acetate (150 ml) and water (150 ml) were added. The aqueous layer was separated and extracted with ethyl acetate (150 ml). The organic fractions were combined, dried over MgSO₄ and evaporated to leave a yellow oil which was purified by flash column chromatography with a 2:1 hexane/ethyl acetate eluent. The title compound was obtained as a yellow solid (2.49 g).

LCMS R_{T} 1.02 mins (not jonised well)

Tlc SiO₂ (1:1 ethyl acetate/hexane) R_{f} 0.24

### Intermediate 37

### 5-Fluoro-2-nitrobenzenecarbothioarnide

To a solution of 2,4-bis(4methoxyphenyl)1,3-dithia-2,4-diphosphetane-2,4-disulfide (2.1 g) in toluene (30 ml), 5-fluoro-2-nitrobenzenecarboamide in toluene (20 ml) was added. The reaction mixture was heated at reflux for 2 hours, then cooled to room temperature. DCM (100 ml) was added and the crude residue was evaporated onto silica gel. The residue was purified by flash column chromatography (solid loading). Elution with hexane/ethyl acetate 4:1 and increasing the polarity to neat ethyl acetate. After evaporation the title compound was obtained as a yellow solid (2.17 g)

LC R_{T} 2.42 mins (not ionised well)

Tlc SiO₂ (1:1 ethyl acetate/hexane) R_{f} 0.42

### Intermediate 38

### 2-(4-Fluoro2-nitrophenyl)-4-methyl-1,3-thiazole

1-Chloro-2-propanone (420 μl) was added dropwise under nitrogen to a stirred solution of 4-fluoro-2-nitrobenzenecarbothioamide (869 mg) in ethanol (16 ml) and the solution heated at reflux for 6 h. The solution was evaporated, treated with aqueous 1M sodium carbonate (25 ml), and extracted with ethyl acetate (2×25 ml). The combined, dried (Na₂SO₄) organic extracts were evaporated, and the residue absorbed from ethyl acetate (20 ml) onto silica gel (Merck 7734, 6 g). The resultant silica gel was applied to a Biotage Flash™, silica column (40 g), and this eluted with ethyl acetate-cyclohexane (1:9) to give the title compound as cream crystals (781 mg).

LC/MS ESI R_{T} 3.21 mins MH⁺ 239.

### Intermediate 39

### 4-Ethyl-2-(5-fluoro-2-nitrophenyl)-1,3-thiazole

To a solution of 5-fluoro-2-nitrobenzenecarbothioamide (1.17 g) in DMF (20 ml), 1-bromo-2-butanone was added and the reaction was heated at reflux for 1.5 hours. The reaction mixture was cooled and partitioned between diethyl ether (100 ml) and water (100 ml). The aqueous layer was extracted with a further portion of diethyl ether (100 ml), then basified to pH 8 with NaOH and extracted with diethyl ether (100 ml) The organics were combined, dried over MgSO₄ and evaporated to leave the a crude yellow oil. The oil was purified by flash column chromatography using 4:1 hexane/ethyl acetate eluent. The title compound was obtained as a yellow oil (1.48 g).

LC/MS ESI R_{T} 3.23 mins MH⁺ 253

### Intermediate 40

### 4-Ethyl-2-(5-hydroxy-2-nitrophenyl-1,3-thiazole

To a solution of 4-ethyl-2-(5-fluoro-2-nitrophenyl)-1,3-thiazole (0.9 g) in DMSO (5 ml) a solution of NaOH (1.3 g) in aqueous DMSO (275 ml; 10% (v/v) H₂O) was added. The reaction mixture was heated at reflux for 1.5 hours then cooled to room temperature. The reaction mixture was evaporated to leave a crude white solid which was purified by flash column chromatography using a 3:1 hexane/ethyl acetate eluent. The title compound was obtained as a white solid (0.8 g).

LC/MS ESI R_{T} 3.19 mins MH⁺ 251

### Intermediate 41

### 5-Fluoro-2-(4-methyl-1,3-thiazol-2-yl)aniline

A solution of 2-(4-fluoro-2-nitrophenyl)-4-methyl-1,3-thiazole (781 mg) in ethanol (20 ml) was added to a suspension of pre-hydrogenated 10% palladium-on-carbon (50% paste with water) (300 mg) in ethanol (35 ml) and hydrogenated at 23° and atmospheric pressure until hydrogen uptake ceased (after 230 ml). The catalyst was filtered off (hyflo) and the filtrate evaporated to give the title compound as cream crystals (600 mg).

LC/MS ESI R_{T} 3.62 mins, MH⁺ 209.

### Intermediate 42

### 2-(4-Ethyl-1,3-thiazol-2-yl)-4-fluoroaniline

A solution of 4-ethyl-2-(5-fluoro-2-nitrophenyl)-1,3-thiazole (0.2 g) in ethanol (2.5 ml) was added to wet Pd(OH)₂ on charcoal under vacuum. The reaction mixture was placed under hydrogen and stirred for 1 hour at room temperature. The crude material obtained was filtered through Celite® and evaporated to leave the title compound as a yellow oil (168 mg).

LC/MS ESI R_{T} 3.70 mins MH⁺ 223

### Intermediate 43

### 2-(4-Ethyl-1,3-thiazol-2-yl)-4-hydroxyaniline GW697266×

A solution of 4-ethyl-2-(5-hydroxy-2-nitrophenyl)-1,3-thiazole (0.2 g) in ethanol (2.5 ml) was added to wet Pd(OH)₂ on charcoal under vacuum The reaction mixture was placed under hydrogen and stirred for 1 hour at room temperature. The crude material obtained was filtered through Celite® and evaporated to leave the title compound as a white solid (0.17 g).

LCMS ESI R_{T} 2.94 mins MH⁺ 221

### Intermediate 44

### tert-Butyl 3-bromo-4-oxopiperidine-1-carboxylate

To a solution of tert-butyl 4-oxopiperidine-1-carboxylate (7.11 g) in diethyl ether (140 ml) was added 5,5-dibromobarbituric acid (5 g). The mixture was stirred for 3 days at room temperature under nitrogen. The reaction was filtered, the filtrate evaporated and the solid purified by flash column chromatography on 230-400 mesh silica Elution with cyclohexane/ethyl acetate 5:1 gave the title compound as a white solid (6.98 g).

Anal. Calcd for C₁₀H₁₆BrNO₃ 0.25 H₂O: C, 42.49; H, 5.88; N, 4.96. Found: C, 42.52; H, 5.61; N, 5.02.

### Intermediate 45

### tert-Butyl 2-(2-aminophenyl)-6,7-dihydror[1,3]thiazolo[5,4-c]pyridine-5(4H)-carboxylate

Tert-butyl 3-bromo-4-oxopiperidine-1-arboxylate (177 mg) in absolute ethanol (5 ml) was added to a solution of 2-aminobenzenecarbothioamide (97 mg) in absolute ethanol (5 ml). The mixture was stirred for 2.25 h at 80° C. under nitrogen and then cooled to room temperature. Triethylamine (0.355 ml) was added, the reaction evaporated in vacuo and the residue partitioned between ethyl acetate (×2) and water. Combined organics were washed with brine, dried over anhydrous magnesium sulphate, filtered and evaporated in vacuo. The residue was purified by Varian Mega Bond Elut® (Si, 5 g); elution with 0-90% dichloromethane in cyclohexane gave the title compound as a yellow residue (41 mg).

LC/MS ESI R_{T} 3.92 mins MH⁺ 332

### Intermediate 46

### 2-(5,6-Dihydro-4H-cyclopentar[d][1,3]thiazol-2-yl)aniline

2-Chlorocyclopentanone (779 mg) in absolute ethanol (32 ml) was added to a solution of 2-aminobenzenecarbothioamide (1 g) in absolute ethanol (32 ml). The mixture was stirred for 2 h at 80° C. under nitrogen, cooled to room temperature and stirred for a fiuter 18 h. Reaction evaporated in vacuo and the residue partitioned between ethyl acetate (×2) and saturated aqueous sodium bicarbonate solution. Combined organics were washed with brine, dried over anhydrous magnesium sulphate, filtered and concentrated in vacuo. The residue was purified by Varian Mega Bond Elut® (Si, 10 g); elution with 0-60% dichloromethane in cyclohexane gave the title compound as a dark yellow solid (104 mg).

LC/MS ESI R_{T} 3.72 mins MH⁺ 217

### Intermediate 66

### tert-Butyl 4(hydroxymethyl)piperidine-1-carboxylate

Triethylaminie (48 ml) was added to a solution of 4-(hydroxymethyl)piperidine (20 g) in dry dichloromethane (100 ml) under nitrogen. Di-tert-butyl dicarbonate (42.4 g) in dry dichloromethane (50 ml) was added dropwise and the mixture was stirred at room temperature for 18 h. The solvent was removed and the residue was partitioned between water (100 ml) and ethyl acetate (100 ml). The organic extracts were washed with water, hydrochloric acid (2M) and brine and were dried (MgSO₄). The solvent was evaporated and the residue was dried under vacuum to give the title compound as a white solid (31.4 g).

MS MH⁺ 216, (Thermospray).

### Intermediate 67

### tert-Butyl (2R,6R)-2,6-dimethyl-4-methylenepiperidine-1-carboxylate

Potassium tert-butoxide (0.83 g) was added in one portion to a suspension of methyl triphenylphosphonium bromide 93.1 g) in dry THF (20 ml) at 0° C. under nitrogen. The mixture was stirred for 20 mins then a solution of tert-butyl (2R,6R)-2,6-dimethyl-4-piperidone-1-carboxylate (CAS 146337-38-4) (1.33 g) in dry THF (5 ml) was added dropwise over 3 mins at 0° C. The mixture was stirred at 0° C. for 0.5 h, then allowed to warm to room temperature and stirred for 16 h. The mixture was partitioned between water (50 ml) and ethyl acetate (3×50 ml). The combined organic extracts were dried (MgSO₄). The solvent was evaporated and the residue pufified by Biotage Flash™, silica. Elution with cyclohexane/ethyl acetate 20:1 gave the title compound as a colourless oil (0.7 g)

Tlc (Cyclohexane/ethyl acetate 20:1) R_{f} 0.20;

### Intermediate 68

tert-Butyl (2R,6R)-4-(hydroxymethyl)-2,6-dimethylpiperidine-1-carboxylate

Borane (1M in THF; 12 ml) was added dropwise to a solution of 2-methyl-2-butene 92.6 ml) at 0oC under nitrogen. The solution was stirred for 1 h at 0° C., then a solution of tert-Butyl (2R,6R)-2,6-dimethyl-4-methylenepiperidine-1-carboxylate (684 mg) in dry THF (5 ml) was added dropwise at 0° C. The mixture was stirred at 0° C. for 0.5 h, then at room temperature for 3 h. Water (0.5 ml), methanol (6 ml) and sodium hydroxide solution (2M; 6 ml) were then sequentially added. The mixture was re-cooled to 0° C., thena hydrogen peroxide (27%; 2.6ml) was added dropwise over 2 mins and the mixture stirred at room temperature for 16 h. The mixture was acidified to pH 4 with HCl (2M; ca 6 ml) then basified to pH 12 with sodium carbonate (2M; ca 10 ml). The mixture was extracted with ethyl acetate (3×20ml) and the combined extracts dried (MgSO₄). The solvent was evaporated to give the title compound as a colourless oil (0.7 g)

NMR (CDCl₃ 400 MHz; δ) 4.20 (1H, m, CH) 3.84 (1H, m, CH) 3.52 (2H, m, CH2) 2.02 (1H, m, CH) 1.75-1.54 (2H, m, CH₂ EQ+AX) 1.48 (9H, s, 3×CH3) 1.28 (3H, d, CH3) 1.20 (4H, d+br d, CH₃+CH EQ) 0.91 (1H, dd, CH AX)

### Intermediate 69

### trans-1-Benzyl-2,6-dimethyl-4-methylenepiperidine

Potassium tert-butoxide (0.27 g) was added in one portion to a suspension of methyl triphenylphosphonium bromide (1.01 g) in dry THF (10 ml) at 0° C. under nitrogen. The mixture was stirred for 20 mins then a solution of trans-2,6-dimethyl-1-(phenylmethyl)-4-Piperidinone (CAS 198211-14-2) (0.41 g) in dry THF (5 ml) was added dropwise over 3 mins at 0oC. The mixture was stirred at 0° C. for 0.5 h, then allowed to warm to room temperature and stirred for 16 h. The mixture was partitioned between water (50 ml) and ethyl acetate (3×50 ml). The combined organic extracts were dried (MgSO₄). The solvent was evaporated and the residue purified by chromatography on silica. Elution with cyclohexane/ethyl acetate 9:1 gave the title compound as a colourless oil (0.4 g)

NMR (CDCl₃ 400 MHz; δ) 7.39 (2H, br d, aromatic 2×CH) 7.29 (2H, br t, aromatic 2×CH) 7.19 (1H, br t, aromatic CH) 4.68 (2H, s, 2×CH) 3.92,3.45 (2H, 2×d, CH₂) 2.92 (2H, m, 2×CH) 2.30 (2H, dd, CH₂) 1.96 (2H, dd, CH₂) 0.99 (6H, d, 2×CH₃)

### Intermediate 70

### [(2alpha,6beta)-1-benzyl-2,6-dimethylpiperidin-4-yl]methanol

Borane (1M in THF; 3.72 ml) was added dropwise to a solution of 2-methyl-2-butene (0.8 ml) at 0° C. under nitrogen. The solution was steeed for 1 h at 0° C., then a solution of trans-1-benzyl-2,6-dimethyl-4-methylenepiperidine (200 mg) in dry THF (2 ml) was added dropwise at 0° C. The mixture was stired at 0° C. for 0.5 h, then at room temperature for 3 h. Water (0.1 ml), methanol (2 ml) and sodium hydroxide solution (2M; 1.9 ml) were then sequentially added. The mixture was re-cooled to 0° C., then hydrogen peroxide (27%; 0.8 ml) was added dropwise over 2 mins and the mixture stirred at room temperature for 16 h. The mixture was acidified to pH 4 with HCl (2M; ca 2 ml) then basified to pH 12 with sodium carbonate (2M; ca 3 ml). The mixture was extracted with ethyl acetate (3×15 ml) and the combined extracts dried (MgSO₄). The solvent was evaporated to give the title compound as a colourless oil (0.272 g)

MS Found MH⁺ 234 (thermospray)

NMR (CDCl₃ 400 MHz; δ) 7.38 (2H, br d, aromatic 2×CH), 7.29 (2H, br t, aromatic 2×CH) 7.21 (1H, br t, aromatic CH) 3.94 (1H, br d, 0.5×CH2) 3.48-3.38 (3H, 2×d, 0.5CH+CH2) 3.02 (1H, m, CH) 2.87 (1H, m, CH) 1.90 (1H, m, CH) 1.64 (1H, br d, CH EQ) 1.42 (1H, br m, CH AX) 1.10-1.05 (2H, m, CH2) 0.95,0.90 (6H, 2×d, 2×CH₃)

### Intermediate 71

### cis-1-Benzyl-2,6-dimethyl-4-methylenepiperidine

Potassuim tert-butoxide (1.91 g) was added in one portion to a suspension of methyl triphenylphosphoniurm iodide (7.14 g) in dry THF (50 ml) at 0° C. under nitrogen. The mixture was stirred for 20 mins then a solution of cis-2,6-dimethyl-1-(phenylmethyl)-4-piperidinone (CAS 198211-15-3) (2.93 g) in dry THF (10 ml) was added dropwise over 2 mins at 0° C. The mixture was stirred at 0° C. for 0.5 h, then allowed to warm to room temperature and stirred for 16 h. The mixture was partitioned between ammonium chloride solution (50 ml) and ethyl acetate (3×50 ml). The combined organic extracts were dried (MgSO₄). The solvent was evaporated and the residue purified by chromatography on silica Elution with ether gave the title compound as a colourless oil (2.6 g)

NMR (CDCl₃ 400 MHz; δ) 7.39 (2H, br d, aromatic 2×CH) 7.29 (2H, br t, aromatic 2×CH) 7.19 (1H, br t, aromatic CH) 4.62 (2H, s, 2×CH) 3.80 (2H, s, CH₂) 2.65 (2H, m, 2×CH) 2.17 (2H, dd, CH₂) 2.05 (2H, br t, CH₂) 1.10 (6H, d, 2×CH₃)

### Intermediate 72

### [(2alpha,4beta,6alpha)-1-benzyl -2,6-dimethylpiperidin-4-yl]methanol isomer 2 (A) and [(2alpha,4alpha,6alpha)-1-benzyl-2,6-dimethylpiperidin-4-yl]methanol isomer 2 (B)

Borane (1M in THF; 25.6 ml) was added dropwise to a solution of 2-methyl-2-butene (5.4 ml) at 0° C. under nitrogen. The solution was stirred for lh at 0° C., then a solution of cis-1-benzyl-2,6-dimethyl-4-methylenepiperidine (1.3 g) in dry THF (10 ml) was added dropwise at 0° C. The mixture was stirred at 0° C. for 0.5 h, then at room temperature for 3 h. Water (0.7 ml), methanol (13 ml) and sodium hydroxide solution (2M; 0.5 ml) were then sequentially added. The mixture was re-cooled to 0° C., then hydrogen peroxide (27%;5.5 ml) was added dropwise over 10 mins and the mixture stirred at room temperature for 16 h. The mixture was acidified to pH 4 with HCl (2M; ca 10 ml) then basified to pH 12 with sodium carbonate (2M; ca 20 ml). The mixture was extracted with ethyl acetate (3×50 ml) and the combined extracts dried (MgSO₄). The solvent was evaporated and the residue purified by chromatography .n silica Elution with dichloromethane/ethanol/ammonia 300:8:1 gave the title compound (A) as a colourless oil (0.461 mg)

NMR (CDCl₃ 400 MHz) 7.38δ (2H, br d, CHₛ), 7.30δ (2H, br t, CHₛ) 7.20δ (1H, br t, aromatic CH) 3.80δ (2H, s, CH₂) 3.60δ (2H, d, CH₂) 2.82δ (2H, m, 2×CH) 1.98δ (1H, m, CH) 1.62-1.50δ (4H, m, 2×CH₂) 1.02δ (6H, d, 2×CH₃)

And the title compound (B) as a colourless oil (134 mg)

NMR (CDCl₃ 400 MHz) 7.38δ (2H, br d, CHₛ), 7.34δ (2H, br t, CH) 7.18δ (1H, br t, aromatic CH) 3.80δ (2H, s, CH₂) 3.43δ (2H, d, CH₂) 2.55δ (2H, m, 2×CH) 1.70δ (2H, br d, CH₂ EQ) 1.14-1.00δ (8H, d+m, 2×CH₃+CH₂ AX)

### Intermediate 80

### tert-Butyl 4-({[({2-[4-methyl-1,3-thiazol-2-yl]phenyl}anuno)carbonyl]oxy}methyl)piperidine-1-carboxylate

A mixture of tert-butyl 4-(hydroxymethyl)piperidine-1-carboxylate (CAS No.123855-51-6; 186 mg) and diisopropylethylamine (0.12 ml) in dry THF (1 ml) was added dropwise to a solution of triphosgene (128 mg) in dry THF (1 ml) at 0-5° C. under nitrogen. The mixture was stirred for 1 hr, then a solution of 2-(4-methyl-1,3-thiazol-2-yl)aniline (164 mg) in THF (1 ml) containing diisopropylethylamine (0.12 ml) was added dropwise and the mixture stirred for 16 hr at room temperature. The nature was treated with 10 ml of saturated sodium bicarbonate solution and 10 ml of water, stirred for 0.5 h, and then extracted into dichloromethane and dried over MgSO₄. The solvent was evaporated and the residue purified by chromatography (Biotage Flash™). Elution with cyclohexane/ethyl acetate (10:1) gave the title compound as a white solid (205 mg.)

LC/MS ESI R_{T} 4.29 mins MH⁺ 432

### Intermediate 81

### tert-Butyl 4-({[({2-[4-trifluoromethyl-1,3-thiazol-2-yl]pbenyl}amino)carbonyl]oxy}methyl) piperidine-1-carboxylate

A mixture of tert-butyl 4-(hydroxymethyl)piperidine-1-carboxylate (CAS No.123855-51-6; 48 mg) and diisopropylethylamine (50 ul) in dry THF (1 ml) was added dropwise to a solution of triphosgene (33 mg) in dry THF (1 ml) at 0-5° C. under nitrogen. Thie mixture was stirred for 1 hr, then a solution of 2-(4-trifuoromethyl-1,3-thiazol-2-yl)aniline (55 mg) in THF (1 ml) containing diisopropylethylamine (50 ul) was added dropwise and the mixture stirred for 16 hr at room temperature. The mixture was treated with Sml of saturated sodium bicarbonate solution and 5 ml of water, stirred for 0.5 h, and then extracted into dichloromethane and dried over MgSO₄. The solvent was evaporated and the residue purified by chromatography (Biotage Flash™). Elution with cyclohexane/ethyl acetate (4:1) gave the title compound as a yellow solid (63 mg.)

LC/MS ESI R_{T} 4.08 mins MH⁺ 486

### Intermediate 82

### tert-Butyl 4-({[({2-[4-cyclopropyl-1,3-thiazol-2-yl]phenyl}amino)carbonyl]oxy}methyl) pineridine-1-

A mixture of tert-butyl 4-hydroxymethyl)piperidine-1-carboxylate (CAS 123855-51-6; 370 mg) and diisopropylethylarne (0.24 ml) in dry THF (5 ml) was added dropwise to a solution of triphosgene (256 mg) in dry THF (3 ml) at 0-5° C. under nitrogen. The mixture was stirred for 1 hr, then a solution of 2-(4-cyclopropyl-1,3-thiazol-2-yl)aniline (371 mg) in THF (5 ml) containing diisopropylethylamine (0.24 ml) was added dropwise and the mixture sti for 16 hr at room temperature. The mixture was treated with 10 ml of saturated sodium bicarbonate solution and 10 ml of water, stirred for 0.5 h, and then extracted into dichloromethane and dried over MgSO₄. The solvent was evaporated and the residue purified by chromatography (Biotage Flash™, silica, 90 g). Elution with cyclohexane/ethyl acetate (6:1) gave the title compound as a white solid (380 mg.)

LC/MS ESI R_{T} 4.37 mins MH⁺ 458

### Intermediate 83

### tert-Butyl 4-{[({[2-(4-phenyl-1,3-thiazol-2-yl)phenyl]amino}carbonyl)oxy]methyl}piperidine-1-carboxylate

A mixture of tert-butyl 4-(hydroxymethyl)piperidine-1-carboxylate (CAS 123855-51-6; 48 mg) and diisopropylethylamine (50 ul) in dry THF (1 ml) was added dropwise to a solution of triphosgene (33 mg) in dry THF (1 ml) at 0-5° C. under nitrogen. The mixture was stirred for 1 hr, then a solution of 2-(4-phenyl-1,3-thiazol-2-yl)aniline (67 mg) in THF (1 ml) containing diisopropylethylamine (50 uL) was added dropwise and the mixture stirred for 16 hr at room temperature. The mixture was treated with 4 m of saturated sodium bicarbonate solution and 4 ml of water, stirred for 0.5 h, and then extracted into dichloromethane and dried over MgSO₄. The solvent was evaporated and the residue purified by chromatography (Biotage Flash™, silica, 40 g). Elution with cyclohexane/ethyl acetate (10:1) gave the title compound as a beige solid (74 mg.).

LC/MS ESI R_{T} 4.62 mins MH⁺ 494

### Intermediate 84

### tert-Butyl 4-{[2-({[2-(4-thien-3-yl -1,3-thiazol-2-yl)phenyl]amino}carbonyl)oxy]methyl}piperidine-1-carboxylate

A mixture of tert-butyl 4-(hydroxymethyl)piperidine-1-carboxylate (CAS.123855-51-6; 48 mg) and diisopropylethylamine (50 uL) in dry THF (1 ml) was added dropwise to a solution of triphosgene (33 mg) in dry THF (1 ml) at 0-5° C. under nitrogen. The mixture was stirred for 1 hr, then a solution of 2-(4-thien-3-yl-1,3-thiazol-2-yl)aniline (68 mg) in THF (1 ml) containing diisopropylethylamine (50 uL) was added dropwise and the mixture stirred for 16 hr at room temperature. The mixture was treated with 4 ml of saturated sodium bicarbonate solution and 4 ml of water, sti for 0.5 h, and then extracted into dichloromethane and dried over MgSO₄. The solvent was evaporated and the residue purified by chromatography (Biotage Flash™, silica, 40 g). Elution with cyclohexane/ethyl acetate (10:1) gave the title compound as an off-white solid (101 mg.).

LC/MS ESI R_{T} 4.59 mins MH⁺ 500.

### Intermediate 85

### tert-Butyl 4-{[({[2-(4-tert-butyl-1,3-thiazol-2-yl)phenyl]amino}carbonyl)oxy]methyl}piperidine-1-carboxUlate

A mixture of tert-butyl 4-(hydroxymethyl)piperidine-1-carboxylate (CAS 123855-51-6; 48 mg) and diisopropylethylamine (50 uL) in dry THF (1 ml) was added dropwise to a solution of triphosgene (33 mg) in dry THF (1 ml) at 0-5° C. under nitrogen. The mixture was stirred for 1 hr, then a solution of 2-(4-tert-butyl-1,3-thiazol-2-yl)aniline (62 mg) in THF (1 ml) containing diisopropylethylamine (50 uL) was added dropwise and the mixture stirred for 16 hr at room temperature. The mixture was treated with 4 ml of saturated sodium bicarbonate solution and 4 ml of water, stirred for 0.5 h, and then extracted into dichloromethane and dried over MgSO₄. The solvent was evaporated and the residue purified by chromatography (Biotage Flash™, silica, 40 g). Elution with cyclohexanie/ethyl acetate (10:1) gave the title compound as a colourless oil (70 mg).

LC/MS ESI R_{T} 4.54 mins MH⁺ 474.

### Intermediate 86

### tert-Butyl 4-{[({[2-(4,5-dimethyl-1,3-thiazol-2-yl)phenyl]amino}carbonyl)oxy]methyl}piperidine-1-carboxylate

A mixture of tert-butyl 4-(hydroxymethyl)piperidine-1-carboxylate (CAS No. 123855-51-6; 48 mg) and diisopropylethylamine (50 uL) in dry THF (1 ml) was added dropwise to a solution of triphosgene (33 mg) in dry THF (1 ml) at 0-5° C. under nitrogen. The mixture was stirred for 1 hr, then a solution of 2-(4,5-dimethyl-1,3-thiazol-2-yl)aniline (54 mg) in THF (1 ml) containing diisopropylethylamine (50 uL) was added dropwise and the mixture stirred for 16 hr at room temperature. The mixture was treated with 4 ml of saturated sodium bicarbonate solution and 4 ml of water, stirred for 0.5 h, and then extracted into dichloromethane and dried over MgSO₄. The solvent was evaporated and the residue purified by chromatography (Biotage Flash™, silica, 40 g). Elution with cyclohexane/ethyl acetate (10:1) gave the title compound as a white solid (55 mg).

LC/MS ESI R_{T} 4.34 mins MH⁺ 446.

### Intermediate 87

### tert-Butyl 4-{[({[2-(4-ethyl-1,3-thiazol-2-yl)phenyl]amino}carbonyl)oxy]methyl}piperidine-1-carboxylate

A mixture of tert-butyl 4-(hydroxymethyl)piperidine-1-carboxylate (CAS 123855-51-6; 48 mg) and diisopropylethylamine (50 uL) in dry THF (1 ml) was added dropwise to a solution of triphosgene (33 mg) in dry THF (1 ml) at 0-5° C. under nitrogen. The mixture was stirred for 1 hr, then a solution of 2-(4ethyl-1,3-thiazol-2-yl)aniline (49 mg) in THF (1 ml) containing diisopropylethylamine (50 uL) was added dropwise and the mixture stirred for 16 hr at room temperature. The mixture was treated with 5 ml of saturated sodium bicarbonate solution and 5 ml of water, stirred for 0.5 h, and then extracted into dichloromethane and dried over MgSO₄. The solvent was evaporated and the residue purified by chromatography (Biotage Flash™, silica, 40 g). Elution with ethyl acetate/cyclohexane (1:6) gave the title compound as a white solid (80 mg).

LC/MS ESI R_{T} 4.26 mins MH⁺ 446.

### Intermediate 88

### tert-Butyl 4-{[({[2-(5-methyl-1,3-thiazol-2-yl)phenyl]amlno}carbonyl)oxy]methyl}piperidine-1-carboxylate

A mixture of tert-butyl 4-(hydroxymethyl)piperidine-1-carboxylate (CAS No. 123855-51-6; 72 mg) and diisopropylethylamine (87 uL) in dry THF (1 ml) was added dropwise to a solution of triphosgene (50 mg) in dry THF (2 ml) at 0-5° C. under nitrogen. The mixture was steeed for 1 hr, then a solution of 2-(5-methyl-1,3-thiazol-2-yl)aniline (64 mg) in THF (2 ml) containing diisopropylethylamine (87 uL) was added dropwise and the mixture stirred for 16 hr at room temperature. The mixture was treated with 5 ml of saturated sodium bicarbonate solution and 5 ml of water, stired for 0.5 h, and then extracted into dichloromethane and dried over MgSO4. The solvent was evaporated and the residue purified by chromatography (Biotage Flash™, silica, 40 g). Elution with ethyl acetate/cyclohexane (1:10) gave the title compound as a white solid (97 mg).

LC/MS ESI R_{T} 4.30 mins MH⁺ 432

### Intermediate 89

### tert-Butyl 4-{[({[2-(4-isopropyl-1,3-thiazol-2-yl)phenyl]amino}carbonyl)oxy]methyl}piperidine-1-carboxylate

A mixture of tert-butyl 4-(hydroxymethyl)piperidine-1-carboxylate (CAS 123855-51-6; 48 mg) and diisopropylethylamine (50 ul) in dry THF (2 ml) was added dropwise to a solution of triphosgene (33 mg) in dry THF (2 ml) at 0-5° C. under nitrogen. The mixture was stirred for 1 hr, then a solution of 2-(4-isopropyl-1,3-thiazol-2-yl)aniline (49 mg) in THF (2 ml) containing diisopropylethylarmine (50 ul) was added dropwise and the mixture stirred for 16 hr at room temperature. The mixture was treated with 4ml of saturated sodium bicarbonate solution and 4 ml of water, stirred for 0.5 h, and then extracted into dichloromethane and dried over MgSO₄. The solvent was evaporated and the residue purified by chromatography (Biotage Flash™, silica, 40 g). Elution with cyclohexane/ethyl acetate (90:10) gave the title compound as a white solid (57 mg.)

LC/MS ESI R_{T} 4.39 mins MH⁺ 460.

### Intermediate 90

### tert-Butyl 4-({[({2-[4-(ethoxycarbonyl)-1,3-thiazol-2-yl]phenyl}amino)carbonyl]oxy}methyl)piperidine-1-carboxylate

Tert-butyl 4-(hydroxymethyl)piperidine-1-carboxylate (CAS-number 123855-51-6; 87 mg) and diisopropylethylamine (74 μL) in tetrahydrofuran (2 ml) was added to a solution of triphosgene (39.7 mg) in tetrahydrofiuran (2 ml) at 5° C. over 2-3 minutes. After stirring for 90 minutes a solution of ethyl 2-(2-aminophenyl)-1,3-thiazole-4-carboxylate (100 mg) and diisopropylethylamine (74 μL) in tetrahydrofiran (2 ml) were added over 30 seconds at 5° C. The mixture was allowed to warm to 20° C. and was stirred for a furler 5 hours. The mixture was evaporated and purified using flash chromatography (SiO₂, hexane:ethyl acetate (6:1)) to give the tide compound (132 mg).

NMR (CDC₃, 400 MHz, δ) 11.55 (1H, br s, NH 8.50 (1H, d, aromatic CH) 8.14 (1H, s, aromatic CH) 7.73 (1H, dd, aromatic CH) 7.44 (dt, aromatic CH) 7.08 (2H, dt, aromatic CH) 4.43 (2H, q, CH₂) 4.13 (2H, br s, CH) 4.08 (2H, d, CH₂) 2.72 (2H, br t, CH₂) 1.91 (1H, m, CH) 1.82 (2H, br d, CH₂) 1.58 (3H, s, CH₃) 1.46 (9H, s, 3CH₃) 1.43 3H, t, CH₃) 1.26 (2H, qd, CH₂)

### Intermediate 91

### tert-Butyl 4-({[({2-[4-(hydroxymethyl)-1,3-thiazol-2-yl]phenyl}amino)carbonyl]oxy}methyl)piperidine-1-carboxylate

A solution of diisopropylethylamine (133 μl) and tert-butyl 4-(hydroxymethyl)piperidine-1-carboxylate (CAS-number 123855-51-6; 157.5 mg) in tetrahydrofuran (3 ml) was added to triphosgene (71.2 mg) in tetrahydrofuran (3 ml) at 3° C. over 5 minutes. After 90 minutes a solution of 2-(2-aminophenyl)-4-(hydroxymethyl)-1,3-thiazole (149.6 mg) and diisopropylethylamine (133 μl) in tetrahydrofuran (3 ml) was added to the cooled solution (0-5° C.) over 5 minutes. The resultant solution was stirred at 0-5° C. for a further 1 hour before allowing to warm to 20° C. and stirring for 18 hours under nitrogen. The mixture was evaporated and partitioned between sodium carbonate (1M, 30 ml) and ethyl acetate (3×30 ml). The combined organics were washed with water (30 ml) and the water back extracted with ethyl acetate (30 ml). The combied organics were dried over magnesium sulphate and evaporated to yield the title compound (296 mg).

NMR (DMSO, 400 MHz, δ) 11.5 (1H, br, s, NH) 8.25 (1H, d, aromatic CH) 7.94 (1H, dd, aromatic CH) 7.63 (1H, s, aromatic CH) 7.53 (1H, td, aromatic CH) 7.24 (1H, td, aromatic CH) 4.70 (2H, s, CH₂) 4.06 (2H, d, CH₂) 4.01 (2H, b, m, CH₂) 2.78 (2H, m, CH₂) 1.90 (1H, m, CH) 1.73 (2H, br d, CH2) 1.45 (9H,s,3CH₃) 1.32 (1H, br dd, CH) 1.17 (2H, br qd, CH₂)

### Intermediate 93

### tert-Butyl 4-[({[(2-{4-[(methylamino)carbonyl]-1,3-thiazol-2-yl}phenyl)amino]carbonyl}oxy)methyl]piperidine-1-carboxylate

A solution of dilsopropylethylamine (55 μl) and tert-butyl 4-(hydroxymethyl)piperidine-1-carboxylate (CAS-number 123855-51-6; 65.3 mg) in tetrahydrofuran (2 ml) was added to triphosgene (30 mg) in tetahydrofuran (2 ml) at 0-5° C. over 10 minutes. After 90 minutes a solution of 2-(2-aminophenyl)-N-methyl-1,3-thiazole-4-carboxamide (70.8 mg) and diusopropylethylamine (55 μl) in tetrahydrofuran (2 ml) was added to the cooled solution (0-5° C.) over 10 minutes. The resultant solution was stirred at 0-5° C. for a further 1 hour before allowing to warm to 20° C. and stirring for 3 days under nitrogen. The mixture was evaporated and partitioned between sodium carbonate (1M, 30 ml) and ethyl acetate (3×30 ml). The combined organics were washed with citric acid (0.5M, 30 ml) which was back extracted with ethyl acetate (30 ml). The combined organics were washed with sodium carbonate (1M, 20 ml) which was back extracted with ethyl acetate (30 ml). The combined organics were dried over magnesium sulphate, evaporated and purified using flash chromatography. Elution with hexane:ethyl acetate (2:1) gave the title compound (18 mg).

NMR (CDCl₃, 400 MHz, δ) 11.2 ((1H, br s, NH) 8.42 (1H, d, aromatic CH) 8.13 (1H, s, aromatic CH) 7.75 (1H, dd, aromatic CH) 7.47 (1H, dt, aromatic CH) 7.11 (dt, aromatic CH) 6.99 (1H, br d, NH) 4.16 (2H, m, CH₂) 4.10 (3H, br d, CH₃) 3.01 (2H, d, CH₂) 2.73 (2H, br t, CH₂) 1.88 (1H, m, CH) 1.76 (2H, br d, CH₂) 1.46 (9H, s, 3CH₃)

### Intermediate 94

### Piperidin-4-ylmethyl 2-[4-(methoxymethyl)-1,3-thiazol-2-yl]phenylcarbamate trifluoroacetate

Tert-butyl 4-({[({2-[4-(methoxymethyl)-1,3-thiazol-2-yl]phenyl}amino)carbonyl]oxy}methyl)piperidine-1-carboxylate (8.5 mg) was dissolved in triiluoroacetic acid (1 ml) and water (0.1 ml) added. The solution formed a suspension after 5 minutes, and was stirred for a further 90 minutes at 20° C. before evaporating to dryness to yield the title compound (10.6 mg).

NMR (CDCl₃, 400 MHz, δ) 11.8 (1H, br s, NH) 9.05 (1H, br s, NH⁺) 8.39 (1H, d, aromatic CH) 8.32 (1H, br s, NH⁺) 7.74 (1H, dd, aromatic CH), 7.41 (dt, aromatic CH) 7.23 (1H, s, aromatic CH)) 7.09 (1H, dt, aromatic CH) 4.64 (2H, s, CH₂) 4.12 (2H, d, CH₂) 3.52 (2H, br d, CH₂) 3.48 (3H, s, CH₃) 2.99 (2H, br q, CH2) 2.05 (3H, br d, CH₃) 1.69 (2H, br q, CH₂)

### Intermediate 95

### tert-Butyl 4-{[({[2-(4-propyl-1,3-thiazol-2-yl)phenyl]amino}carbonyl)oxy]methyl}piperidine-1-carboxylate

A mixture of tert-butyl 4-(hydroxymethyl)piperidine-1-carboxylate (680 mg) and diisopropylethylamine (1.65 ml) in dry THF (10 ml) was added dropwise to a solution of triphosgene (284 mg) in dry THF (5 ml) at 0-5° C. under nitrogen. The mixture was stirred for 3 h, then a solution of 2-(4-propyl-1,3-thiazol-2-yl)aniline (626 mg) in dry THF (5 ml) was added dropwise and the mixture stirred for 16 h at room temperature. Water (10 ml) followed by ethyl acetate 10 ml) were added to the reaction. The aqueous phase was extracted with ethyl acetate (10 ml). The combined organics were washed with brine (15 ml) and dried (MgSO₄). The solvent was evaporated and the residue purified by Varian Mega Bond Elut®; on silica. Elution with 10% dichloromethane/cyclohexane followed with 10% ethyl acetate gave the title compound as a white solid (1.068 g)

LC/MS ESI R_{T} 4.40 mins MH⁺ 460

Tlc SiO₂ (Cyclohexane/Ethyl acetate 1:8) R_{f} 0.19

### Intermediate 96

### tert-Butyl 4-{[({[2-(4-pentyl-1,3-thiazol-2-yl)phenyl]amino}carbonyl)oxy]methyl}piperidine-1-carboxylate and tert-butyl 4-{[({[2-(5-butyl-4-methyl-1,3-thiazol-2-yl)phenyl]amino}carbonyl)oxy]methyl}piperidine-1-carboxylate

A mixture of tert-butyl 4-(hydroxymethyl)piperidine-1-carboxylate (723 mg) and diisopropylethylamine (1.75 ml) in dry THF (10 ml) was added dropwise to a solution of triphosgene (302 mg) in dry THF (5 ml) at 0-5° C. under nitrogen. The mixture was stirred for 1.5 h, then a solution of a mixture of 2-(4-pentyl-1,3-thiazol-2-yl)aniline and 2-(5-butyl-4-methyl-1,3-thiazol-2-yl)aniline (760 mg) in dry THF (5 ml) was added dropwise. The mixture was stirred for 7 days at room temperature. Water (10 ml) followed by ethyl acetate (10 ml) were added to the reaction. The aqueous phase was extracted with ethyl acetate (10 ml). The combined organics were washed with brine (20 ml) and dried (Na₂SO₄). The solvent was evaporated and the residue purified by flash column chromatography on silica. Elution with cyclohexane/ethyl acetate 8:1 afforded the title compounds as a yellow oil (1.1 g)

LC/MS ESI R_{T} 4.65 mins MH⁺ 488

LC/MS ESI R_{T} 4.52 mins MH⁺ 488

Tlc SiO₂ (Cyclohexane/Ethyl acetate 8;1) R_{f} 0.16

### Intermediate 97

### tert-Butyl 4-{[({[2-(4-butyl-1,3-thiazol-2-yl)phenl]amino}carbonyl)oxy]methyl}piperidine-1-carboxylate

A mixture of tert-butyl 4-(hydroxymethyl)piperidine-1-carboxylate (690 mg) and diisopropylethylamine (1.5 ml) in dry THF (10 ml) was added dropwise to a solution of triphosgene (288 mg) in dry THF (5 ml) at 0-5° C. under nitrogen. The mixture was stired for 3 h, then a solution of 2-(4-butyl-1,3-thiazol-2-yl)aniline (677 mg) in dry THF (5 ml) was added dropwise. The mixture was stirred for 16 h at room temperature. Water (10 ml) followed with ethyl acetate (10 ml) were added to the reaction. The aqueous phase was extracted with ethyl acetate (10 ml). The combined organics were washed with brine (10 ml) and dried (Na₂SO₄). The solvent was evaporated and the residue purified by Biotage Flash™ on silica. Elution with dichloromethane followed by ethylacetate gave the title compound as a pale yellow powder (660 mg)

LC/MS ESI R_{T} 4.11 mins MH⁺ 474

Tlc SiO₂ (Dichloromethane) Rf 0.1

### Intermediate 98

### tert-Butyl 4-[({[(2-{4-methyl-5-[(methylamino)carbonyl]-1,3-thiazol-2-yl}phenyl)amino]carbonyl}oxy)methyl]piperidine-1-carboxylate

A minxture of tert-butyl 4-(hydroxymethyl)piperidine-1-carboxylate (43 mg) and diisopropylethylamine (0.095 ml) in dry THF (3 ml) was added dropwise to a solution of triphosgene (16 mg) in dry THF (5 ml) at 0-5° C. under nitrogen. The mixture was stirred for 3 h, then a solution of 2-(2-aminophenyl)-N,4dimethyl-1,3-thiazole-5-carboxamide (45 mg) in dry THF (35 ml) was added dropwise. The mixture was stirred for 16 h at room temperature. Water (5 ml) followed with ethyl acetate (8 ml) were added to the reaction. The aqueous phase was extracted with ethyl acetate (8 ml). The combined organics were washed with brine (10 ml) and dried (Na₂SO₄). The solvent was evaporated and the residue purified by using a Varian Mega Bond Blut® 10 g silica solid phase extraction cartridge with 1:1 ethyl acetate/cyclohexane as the eluent. The material was re-purified by Biotage Flash™, on silica Elution with 1:1 cyclohexane/ethyl acetate gave the tide compound as a yellow oil (50 mg)

LC/MS ESI R_{T} 3.81 mins MH⁻ 487

Tlc SiO₂ (cyclohexane/ethyl acetate, 1:1) R_{f} 0.23

### Intermediate 99

### tert-Butil 4-[({[(2-{4-[2-(benzyloxy)ethyl]-1,3-thiazol-2-yl}phenyl)amino]carbonyl}oxy)methyl]piperidine-1-carboxylate

Diisopropylethylamine (0.57 ml) was added to a solution of tripbosgene (320 mg) in dry THF (2.5 ml) at 0-5° C. under nitrogen. After stirring for 2 minutes, a solution of tert-butyl 4-(hydroxymethyl)piperidine-1-carboxylate (694 mg) in dry THF (3 ml) was added and the resulting mixture was stirred for 2 hours 30 mins at 0-5° C. A solution of 2-{4-[2-(benzyloxy)ethyl]-1,3-thiazol-2-yl}aniline (1 g) in dry THF (7 ml) and diisopropylethylamine (0.57 ml) were then successively added. The mixture thus obtained was stirred for 16 hours at room temperature then partitioned between ethyl acetate (200 ml) and saturated aqueous sodium bicarbonate (150 ml). The aqueous layer was separated, extracted with ethyl acetate (100 ml). The organic extracts were combined, dried (MgSO₄) and evaporated. The resulting residue was purified by flash column, chromatography. Elution with hexane/ethyl acetate 4:1 gave the title compound as a pale yellow solid (1.27 g).

LC/MS ESI R_{T} 4.47 min⁺ 552.3

Tlc SiO₂ (Hexane/Ethyl acetate 4:1) R_{f} 0.18

### Intermediate 100

### tert-Butyl 4-{[({[2-(4-formyl-1,3-thiazol-2-yl)phenyl]amino}carbonyl)oxy]methyl}piperidine-1-carboxylate

To a solution of oxalyl chloride (0.136 ml) in dichloromethane (1 ml) was added DMSO (0.259 ml) at −78° C. After stirring for one hour at that temperature, a solution of tert-butyl 4-({[({2-[4-(hydroxymethyl)-1,3-thiazol-2-yl]phenyl}amino)carbonyl]oxy}methyl)piperidine-1-carboxylate (465 mg) in dichloromethane (4 ml) was added dropwise. Thirty minutes later, triethylamine (1 ml) was added and the resulting solution was strred for one hour at −78° C. then allowed to warm-up slowly to room temperature. The reaction mixture was partitioned between dichloromethane (50 ml) and water (20 ml). The organic layer was separated, washed with 0.5 M hydrochloric acid (20 ml) then saturated aqueous sodium bicarbonate (20 ml) before drying (MgSO₄). After evaporation, the title compound was obtained as a white solid (450 mg).

LC/MS ESI R_{T} 3.78 mins MH⁺ 446.5

Tlc SiO₂ (Hexane/Ethyl acetate 1:1) R_{f} 0.44

### Intermediate 101

### tert-Butyl 4-({[({2-[4-(difluoromethyl)-1,3-thiazol-2-yl]phenyl}amino)carbonyl]oxy}methyl)Riperidine-1-carboxylate

To a solution of tert-butyl 4-{[({[2-(4-formyl-1,3-thiazol-2-yl)phenyl]amino}carbonyl)oxy]methyl}piperidine-1-carboxylate (150 mg) in dichloromethane (0.5 ml) was added (diethylamino)sulfur trifluoride (0.065 ml) at 0° C. After stirring at room temperature for 18 hours, the reaction mixture was partitioned between dichloromethane (40 ml) and water (10 ml). The aqueous phase was separated and extracted with dichloromethane (10 ml). The combined organic extracts were washed with saturated aqueous sodium bicarbonate (20 ml), dried (MgSO₄) and evaporated to give a crude material which was purified by flash chromatography. Elution with ethyl acetatelhexane 3:1 gave the title compound as a white solid (85 mg).

LC/MS ESI R_{T} 3.93 mins MH⁺ 467.5

Tlc SiO₂ (Hexane/Ethyl acetate 1:1) R_{f} 0.57

### Intermediate 102

### tert-Butyl 4-({[({2-[(4-fluoromethyl)-1,3-thiazol-2-yl]phenyl}amino)carbonyl]oxy}methyl)piperidine-1-carboxylate

To a solution of 4-({[({2-[4-(hydroxymethyl)-1,3-thiazol-2-yl]phenyl}aminuo)carbonyl]oxy}methyl)piperidine-1-carboxylate (150 mg) in dichloromethane (0.5 ml) was added (diethylamino)sulfur trifluoride (0.046 ml) at 0° C. After sting at room temperature for 3 hours 20 mins, more (diethylamino)sulfur trifluoride (0.015 ml) was added. After stirring for another 16 hours, the reaction mixture was partitioned between dichioromethane (40 ml) and water (10 ml). The aqueous phase was separated and extracted with dichloromethane (10 ml). The combined organic extracts were washed with saturated aqueous sodium bicarbonate (20 ml), dried (MgSO₄) and evaporated to give a crude material which was purified by flash chromatography. Elution with ethyl acetate/hexane 3:1 gave the tide compound as a white solid (47 mg).

LC/MS ESI R_{T} 3.90 mins MH⁺ 450.0

Tlc SiO₂ (Hexane/Ethyl acetate 1:1) R_{f} 0.55

### Intermediate 103

### tert-Butyl 4-({[({2-[4-(1-hydroxyethyl-1,3-thiazol-2-yl]phenyl}amino)carbonyl]oxy}methyl)piperidine-1-carboxylate

To a solution of tert-butyl 4-{[({[2-(4-formyl-1,3-thiazol-2-yl)phenyl]amino}carbonyl)oxy]methyl}piperidine-1-carboxylate (150 mg) in THF (3 ml) at −78° C. was added a 3N solution of methyl magnesium chloride in THF (0.27 ml). The resulting solution was stirred and allowed to warm up to room temperature over 16 hours. After quenching with water (1 ml), the mixture was partitioned between with dichloromethane (200 ml) and water (50 ml). The organic phase was separated, washed with saturated aqueous sodium bicarbonate, dried (MgSO₄) and evaporated to give the tile compound as a pale yellow solid (135 mg).

LC/MS ESI R_{T} 3.77 mins 462.6

Tlc SiO₂ (Hexane/Ethyl acetate 1:1) R_{f} 0.34

### Intermediate 104

### (R)-tert-Butyl 4-({[({2-[4-(1-hydroxyethyl)-1,3-thiazol-2-yl]phenyl}amino)carbonyl]oxy}methyl)piperidine-1-carboxylate

Diisopropylethylamine (0.23 ml) was added to a solution of triphosgene (128 mg) in dry THF (3 ml) at 0-5° C. under nitrogen. After stirring for 10 minutes, a solution of tert-butyl 4-(hydroxymethyl)piperidine-1-carboxylate (282 mg) in dry THF (5 ml) was added and the resulting mixture was stirred for 1 hour at 0-5° C. A solution of (R)-2-[4(1-hydroxyethyl)-1,3-thiazole-2-yl]aniline (330 mg) in dry THF (5 ml) and diisopropylethylamine (0.23 ml) were then successively added. The mixture thus obtained was stirred for 16 hours at room temperature then partitioned between ethyl acetate (150 ml) and water (50 ml). The aqueous layer was separated and extracted with ethyl acetate (100 ml). The organic extracts were combined, washed with saturated aqueous sodium bicarbonate (20 ml), dried (MgSO₄) and evaporated to give a residue which was purified by flash column chromatography. Elution with hexane/ethyl acetate 3:1 gave the title compound as white solid (475 mg).

LC/MS ESI R_{T} 3.87 mins MH⁺ 462.6

Tlc SiO₂ (Hexane/Ethyl acetate 1:1) R_{f} 0.34

### Intermediate 105

### tert-Butyl 4-{[({[2-(4-acetyl-1,3-thiazol-2-yl)phenyl]amino}carbonyl)oxy]methyl}piperidine-1-carboxylate

To a solution of oxalyl chloride (0.024 ml) in dichloromethane (2 ml) was added DMSO (0.041 ml) at −78° C. After sting for one hour at that temperature, a solution of tert-butyl 4-({[({2-[4-(1-hydroxyethyl)-1,3-thiazol-2-yl]phenyl}amino)carbonyl]oxy}methyl)piperidine-1-carboxylate (83 mg) in dichloromethane (1.5 ml) was added dropwise. Fifteen minutes later, triethylane (0.18 ml) was added and the resulting solution was sdrred for one hour at −78° C. then allowed to warm-up slowly to room temperature over 3 hours. The reaction mixure was partitioned between dichloromethane (50 ml) and water (20 ml). The organic layer was separated, washed with 0.5 M hydrochloric acid (20 ml) and saturated aqueous sodium bicarbonate (20 ml) then dried (MgSO₄). After evaporation, the title compound was obtained as a white solid (85 mg).

LC/MS ESI R_{T} 3.92 mins MH⁺ 460.6

Tlc SiO₂ (Hexane/Ethyl acetate 1:1) R_{f} 0.58

### Intermediate 106

### tert-Butyl 4-({[({2-[4-(1,1-difluoroethyl)-1,3-thiazol-2-yl]phenyl}amino)carbonyl]oxy}methyl)piperidine-1-carboxylate

To a solution of tert-butyl 4-{[({[2-(4-acetyl-1,3-thiazol-2-yl)phenyl]amino}carbonyl)oxy]methyl}piperidine-1-carboxylate (81 mg) in dichloromethane (1 ml) was added diethyl amino sulfur tifluoride (0.45 ml). After stirring at room temperature for 24 hours, more diethyl amino sulfur trifluoride (0.45 ml) was added. After stirring for a further 4 days, the reaction mixture was partitioned between dichloromethane (50 ml) and water (10 ml). The aqueous phase was separated and extracted with dichloromethane (20 ml). The combined organic extracts were washed with saturated aqueous sodium bicarbonate (20 ml), dried (MgSO₄) and evaporated to give a crude material which was purified by flash chromatography. Elution with ethyl acetatejhexane 4?3:1 gave the title compound as apale yellow solid (40 mg).

LC/MS ESI R_{T} 4.11 mins MH⁺ 480.2

### Intermediate 107

### tert-Butyl 4-({[({2-[4-(2-ethoxy-2-oxoethyl)-1,3-thiazol-2-yl]phenyl}amino)carbonyl]oxy}methyl)piperidine-1-carboxylate

Diisopropylethylamine (0.57 ml) was added to a solution of triphosgene (320 mg) in dry THF (2.5 ml) at 0-5° C. under nitrogen. After stirring for 2 minutes, a solution of tert-butyl 4-(hydroxymethyl)piperidine-1-carboxylate (698 mg) in dry THF (6 ml) was added and the resulting mixture was stirred for 1 hour at 0-5° C. A solution of ethyl [2-(2-aminophenyl)-1,3-thiazol-4-yl]acetate (0.85 g) in dry THF (5 ml) and diisopropylethylamine (0.57 ml) were then successively added. The mixture thus obtained was stirred for 16 hours at room temperature then partitioned between ethyl acetate (200 ml) and saturated aqueous sodium bicarbonate (150 ml). The aqueous layer was separated and extracted with ethyl acetate (100 ml). The organic extracts were combined, dried (MgSO₄) and evaporated to give a residue which was purified by flash column chromatography. Elution with hexane/ethyl acetate 4:1 gave the title compound as pale yellow solid (0.92 g).

LC/MS ESI R_{T} 4.12 mins MH⁺ 504.3

Tlc SiO₂ (Hexane/Ethyl acetate 4:1) R_{f} 0.11

### Intermediate 108

### tert-Butyl 4-({[({2-[4-(2-hydroxyethyl)-1,3-thiazol-2-yl]phenyl}amino)carbonyl]oxy}methyl)piperidine-1-carboxylate

To solution of tert-butyl 4-({[({2-[4-(2-ethoxy-2-oxoethyl)- 1,3-thiazol-2-yl]phenyl}amino)carbonyl]oxy}methyl)piperidine-1-carboxylate (822 mg) in THF (10 ml) was added lithium borohydride (35 mg). After stirring at room temperature for 6 hours, more lithium borohydride (35 mg) was added and the resulting mixture was stirred at room temperature for 15 hours. Methanol (10 ml) was then added and the mixture was stirred for 10 mins. The solvents were evaporated and the residue was partitioned between ethyl acetate (150 ml) and water (50 ml). The aqueous phase was separated and extracted with ethyl acetate (50 ml). The organic extracts were combined, dried (MgSO₄) and evaporated to a pale yellow solid (765 mg). A portion of this solid (610 mg) was purified by flash column chromatography. Elution with ethyl acetate/cyclohexane 1:1 afforded the title compound as white solid (515 mg).

LC/MS ESI R_{T} 3.84 mins MH⁺ 462.2

### Intermediate 109

### tert-Butyl 4-({[({2-[4-(2-fluoroethyl)-1,3-thiazol-2-yl]phenyl}amino)carbonyl]oxy}methyl)piperidine-1-carboxylate

To solution of tert-butyl 4-({[({2-[4-(2-hydroxyethyl)-1,3-thiazol-2-yl]phenyl}amino)carbonyl]oxy}methyl)piperldine-1-carboxylate (68 mg) in dichloromethane (0.5 ml) was added (diethylamino)sulphur trifluoride (0.40 ml). After stirring for 6 hours and 10 mins at room temperature, more (diethylamino)sulphur trifluoride (0.40 ml) and dichloromethane (0.5 ml) were added. The resulting solution was stirred at room temperature for 21 hours. The mixture was diluted with dichloromethane (100 ml) and washed with aqueous saturated sodium bicarbonate (50 ml). The aqueous phase was separated and extracted with dichloromethane (100 ml). The organic extracts were combined, dried (MgSO₄) and evaporated to give a crude oil which was purified by flash column chromatography. Elution with ethyl acetate/cyclohexane 4:1 afforded the title compound as a pale yellow solid (57 mg).

LC/MS ESI R_{T} 4.15 mins MH⁺ 464.2

Tlc SiO₂ (Hexane/Ethyl acetate 4:1) R_{f} 0.20

### Itermediate 110

### tert-Butyl 4-({[({2-[4-(2,2-difluoroethyl)-1,3-thiazol-2-yl]phenyl}amino)carbonyl]oxy}methyl)piperidine-1-carboxylate

Diisopropylethylamine (0.034 ml) was added to a solution of triphosgene (19 mg) in dry THF (0.2 ml) at 0-5° C. After stirrng for 5 minutes, a solution of tert-butyl 4-(hydroxymethyl)piperidine-lcarboxylate (41.1 mg) in dry THF (0.5 ml) was added and the resulting mixture was stirred for 1 hour 25 mins. A solution of 2-[4-(2,2-difluoroethyl)-1,3-thiazol-2-yl]aniline in THF (0.5 ml) and diisopropylethylarine (0.034 ml) were successively added and the mixture thus obtained was stirred for 16 hours from 0° C. to room temperature. The reaction mixture was then partitioned between ethyl acetate (30 ml) and saturated aqueous sodium bicarbonate (20 ml). The aqueous phase was separated and extracted with ethyl acetate (20 ml). The organic extracts were combined, dried (MgSO₄) and evaporated. The crude residue was purified by flash chromatography. Elution with ethyl acetate/cyclohexane 1:3 gave the title compound (42 mg).

LC/MS ESI R_{T} 4.20 mins MH⁺ 482.5

### Intermediate 111

### tert-Butyl 4-{[({[2-(4-cyclobutyl)-1,3-thiazol-2-yl)phenyl]amino}carbonyl)oxy]methyl}piperidine-1-carboxylate

A solution of tert-butyl 4-(hydroxymethyl)piperidine-1-carboxylate (CAS-number 123855-51-6, 176 mg) and N,N-diisopropylethylamine (439 μl) in dry tetrahydrofuran (3 ml) was added dropwise to a cooled (0° C.) solution of triphosgene (122 mg) in dry tetrahydrofuran (7 ml) under an atmosphere of nitrogen. The resulting solution was stred at room temperature for 1.5 h and then cooled to 0° C. once more. A solution of 2-(4-cyclobutyl-1,3-thiazol-2-yl)aniline (188 mg) in dry tetrahydrofuran (1 ml) was added and the mixture was sired at room temperature for 16 h. Water was added and after 4 hr the mixture was extracted with ethyl acetate. The combined organic extracts were washed with brine and dried (Na₂SO₄). The solvent was removed and the residue was purified by column chromatography on silica. Elution with cyclohexane/ethyl acetate 8:1 gave the title compound as a yellow oil which solidified on standing (285 mg).

LC/MS ESI R_{T} 4.56 mins MH⁺ 472

### Intermediate 112

### tert-Butyl 4-{[({[2-(4-cyclohexyl-1,3-thiazol-2-yl)phenyl]amino}carbonyl)oxy]methyl}piperidine-1-caboxylate

A solution of 2-(4-cyclohexyl-1,3-thiazol-2-yl)aniline (155 mg) and N,N-diisopropylethylamine (314 μl) in dry tetrahydrofuran (2 ml) was added dropwise to a cold (0° C.) solution of tripbosgene (94 mg) in tetrahydrofuran (5 ml) under an atmosphere of nitrogen and the solution was stirred at 0° C. for 10 mins. A solution of tert-butyl 4-(hydroxymethyl)piperidine-1-carboxylate (CAS-number 123855-51-6, 129 mg) in dry tetrahydroftran (1 ml) was added and the resulting solution was heated at 70° C. for 3 days. Sodium bicarbonate (8%)/water 1:1 and dichloromethane were added and the resulting mixture was stirred vigorously for 1.5 h. The reaction mixture was partitioned between the two phases and the combined organic extracts were washed with brine and dried (Na₂SO₄). The solvent was removed and the residue was purified by column chromatography on silica using cyclohexane/ethyl acetate (9:1) as eluant. This gave the title compound (63 mg).

LC/MS ESI R_{T} 4.75 mins MH⁺ 500

### Intermediate 113

### tert-Butyl 4-{[({[2-(4-cyclopentyl-1,3-thiazol-2-yl)phenyl]amino}carbonyl)oxy]methyl}piperidine-1-caboxylate

A solution of tert-butyl 4-(hydroxymethyl)piperidine-1-carboxylate (CAS-number 123855-51-6, 54 mg) and N,N-diisopropylethylamine (130 μl) in dry tetrahydrofuran (2 ml) was added dropwise to a cooled (0° C.) solution of triphosgene (38 mg) in dry tetrydrofuran (4 ml) under an atmosphere of nitrogen. The resulting solution was stirred at room temperature for 1.5 h and then cooled to 0° C. once more. A solution of 2-(4-cyclopentyl-1,3-thiazol-2-yl)aniline (60 mg) in dry tetrahydrofuran (1 ml) was added and the mixture was std at room temperature for 3 days. Sodium bicarbonate (8%)/water 1:1 was added and after 1.5 hr the mixture was extracted with ichloromethane. The combined organic extracts were washed with brine and dried Na₂SO₄). The solvent was evaporated and the residue was purified by colurmn chromatography on silica. Elution with cyclohexane/ethyl acetate 9:1 gave the title compound as a white solid (26 mg).

LC/MS ESI R_{T} 4.79 mins MH⁺ 486

### Intermediate 114

### tert-Butyl 4-{[({2-[4-(cyclopropylmethyl)-1,3-thiazol-2-yl]phenyl}amino)carbonyl]oxy}methyl)piperidine-1-caboxylate

A mixture of tert-butyl 4-(hydroxymethyl)piperidine-1-carboxylate (CAS-number 123855-51-6; 231 mg) and diisopropylethylamine (0.375 ml) in dry THF (7.5 ml) was added dropwise to a solution of triphosgene (160 mg) in dry THF (7.5 ml) at 0-5° C. under nitrogen. The mixture was stirred for 1 h at room temperature, re-cooled to 0-5° C., then a solution of 2-[4-(cyclopropylmethyl)-1,3-thiazol-2-yl]aniline (248 mg) in dry THF (7.5 ml) containing diisopropylethylamine (0.187 ml) was added dropwise and the mixture stirred for 18 h at room temperature. The reaction was treated with saturated aqueous sodium bicarbonate solution (30 ml) and extracted with dichloromethane (×3). Combined organics were washed with brine, dried over anhydrous magnesium sulphate, filtered and evaporated in vacuo. The residue was purified by Varian Mega Bond Elut® (Si, 10 g); elution with 0-65% dichloromethane in cyclohexane followed by 100% dichloromethane gave the title compound as a white solid (162 mg).

LC/MS ESI R_{T} 4.55 mins MH⁺ 472

### Intermediate 115

### tert-Butyl 4-{[({[2-(4-isobutyl-1,3-thiazol-2-yl)phenyl]amino}carbonyl)oxy]methyl}piperidine-1-carboxylate

A mixture of tert-butyl 4-(hydroxymethyl)piperidine-1-carboxylate (CAS-number 123855-51-6; 187 mg) and diisopropylethylamine (0.304 ml) in dry THF (6 ml) was added dropwise to a solution of triphosgene (130 mg) in dry THF (6 ml) at 0-5° C. under nitrogen. The mixture was stieed for 1 h at room temperature, re-cooled to 0-5° C., then a solution of 2-(4-isobutyl-1,3-thiazol-2-yl)aniline (203 mg) in dry THF (6 ml) containing diisopropylethylamine (0.152 ml) was added dropwise and the mixture stirred for 72 h at room temperature. The reaction was treated with saturated aqueous sodium bicarbonate solution (30 ml) and extracted with dichloromethane (×3). Combined organics were washed with brine, dried over anhydrous magnesium sulphate, filtered and evaporated in vacuo. The oil was purified by Varian Mega Bond Elut® (Si, 10 g); elution with 0-65% dichloromethane in cyclohexane followed by 100% dichloromethane gave the title compound as a white solid (148 mg).

LC/MS ESI R_{T} 4.69 mins MH⁺ 474

### Intermediate 116

### tert-Butyl 2-{2-[({[1-(tert-butoxycarbonyl)piperidin-4-yl]methoxy}carbonyl)amino]phenyl}-6,7-dihydro[1,3]thiazol[5.4-c]pyridine-5(4H)-carboxylate

A mixture of tert-butyl 4-(hydroxymethyl)piperidine-1-carboxylate (CAS-number 123855-51-6; 26.6 mg) and diisopropylethylamine (0.043 ml) in dry THF (1 ml) was added dropwise to a solution of triphosgene (18.5 mg) in dry THF (1ml) at 0-5° C. under nitrogen . The mixture was stirred for 1 h at room temperature, re-cooled to 0-5° C., then a solution of tert-butyl 2-(2-aminophenyl)-6,7-dihydro[1,3]thiazolo[5,4c]pyridine-5(4H)-carboxylate (41 mg) in dry THF (2 ml) containing dilsopropylethylamine (0.022 ml) was added dropwise and the mixture stirred for 20 h at room temperature. The reaction was treated with saturated aqueous sodium bicarbonate solution and extracted with dichloromethane (×2). Combined organics were washed with brine, dried over anhydrous magnesium sulphate, filtered and evaporated in vacuo. The residue was purified by Varian MegaBond Elut® (Si, 1 g); elution with 0-90% dichloromethane in cyclohexane gave the title compound as a light yellow residue (27 mg)

LC/MS ESI R_{T} 4.56 min MH⁺ 573

### Intermediate 117

### tert-Butyl 4-{[({[2-(5.6-dihydro-4H-cyclopenta[d][1,3]thiazol-2-yl)phenyl]amino}carbonyl)oxy]methyl}piperidine-1-carboxylate

A mixture of tert-butyl 4-(hydroxymethyl)piperidine-1-carboxylate (CAS-number 123855-51-6; 104 mg) and diisopropylethylamine (0.168 ml) in dry THF (5 ml) was added dropwise to a solution of triphosgene (72 mg) in dry THF (5 ml) at 0-5° C. under nitrogen. The mixture was stirred for 1 h at room temperature, re-cooled to 0-5° C., then a solution of 2-(5,6-dihydro-4H-cyclopenta[d][1,3]thiazol-2-yl)aniline (104 mg) in dry THF (5 ml) contaitilug diisopropylethylamine (0.084 ml) was added dropwise and the miuture stirred for 20 h at room temperature. The reaction was treated with saturated aqueous sodium bicarbonate solution and extracted with dichloromethane (×2). Combined organics were washed with brine, dried over anhydrous magnesium sulphate, filtered and evaporated in vacuo. The residue was purified by Varian Mega Bond Elut® (Si, 5 g); elution with 0-90% dichloromethane in cyclohexane gave the title compound as a light yellow solid (162 mg).

LC/MS ESI R_{T} 4.55 mins MH⁺ 458

### Intermediate 118

### tert-Butyl 4-[({[(2-bromophenyl)amino]carbonyl}oxy)methyl]piperidine-1-carboxylate

A solution of tert-butyl 4-(hydroxymethyl)piperidine-1-carboxylate (CAS-number 123855-51-6, 2.5 g) in dry tetrahydrofiran (10 ml) was added dropwise to a cooled (0° C.) solution of triphosgene (1.69 g) in dry tetrahydrofuran (80 ml) under an atmosphere of nitrogen. N,N-diisopropylethylamine (3 ml) was added dropwise and the resulting solution was stirred at room temperature for 1.5 h and then cooled to 0° C. once more. A solution of o-bromoaniline (2 g) and N,N-diisopropylethylamine (3 ml) in dry tetrahydrofuran (10 ml) was added and the mixture was stirred at room temperature for 3 days. Water was added followed by sodium bicarbonate (8%) and after lhr the mixture was extracted with dichloromethane. The combined organic extracts were washed with brine and dried (Na₂SO₄). The solvent was removed and the residue was purified by column chromatography on silica Elution with cyclohexane/ethyl acetate 10:1 gave the title compound (2.3 g)

LC/MS ESI R_{T} 3.80 mins MH⁺ 413,415

### Intermediate 119

### tert-Butyl 4-{[({[2-(4,4,5,5-tetramethyl-1,3,2dioxaborolan-2-yl)phenyl]amino}carbonyl)oxy]methyl}piperidine-1-carboxylate

A solution of bis(pinacolato)diboron (623 mg) in dry dimethoxyethane (2 ml) was added to a solution of tert-butyl 4-[({[(2-bromophenyl)amino]carbonyl}oxy)methyl]piperidine-1-carboxylate. Potassium acetate (602 mg) and 1,1′-bis(diphenylphosphino)ferrocene-palladium dichloride (167 mg) were added and the resulting mixture was heated at 80° C. for 16 h. The mixture was partitioned between brine and dichloromethane and the combined organic extracts were dried (Na₂SO₄) and concentrated in vacuo to give a brown oil. This residue was purified (Varian Mega Bond Elut®) using cyclohexane/ethyl acetate 7:1 as eluant to, give the title compound as a colourless oil (454 mg).

LC/MS ESI R_{T} 4.21 mins MH⁺ 461

### Intermediate 120

### tert-Butyl 4-{[({[2-(1,3-thiazol-2-yl)phenyl]amino}carbonyl)oxy]methyl}piperidine-1-carboxylate

2-Bromothiazole (0.59 ml) was added to a solution of tert-butyl 4-{[({[2-(4,4,5,5-tehethyl-1,3,2-dioxaborolan-2-yl)phenyl]amino}carbonyl)oxy]methyl}piperdine-1-carboxylate (1.0 g) in dry dimethoxyethane (20 ml, pretreated with activated alumina). Triethylamine (0.92 ml) was added followed by tetrakis(triphenylphosphino)palladium (0) (254 mg) and water (2 ml). The resulting reaction mixture was heated at 88° C. for 16 h. After cooling the reaction was concentrated in vacuo and the residue was partitioned between brine and ethyl acetate. The combined organic extracts were dried (Na₂SO₄) and the solvent was removed to give an oil which was purified by column chromatography on silica. Elution with cyclohexane/ethyl acetate (6:1) gave the title compound as a colourless oil (120 mg).

LC/MS ESI R_{T} 4.17 mins MH⁺ 418

### Bromo Intermediate

### tert-Butyl 4-{[({[2-(4-bromo-1,3-thiazol-2-yl)phenyl]amino}carbonyl)oxy]methyl}piperidine-1-carboxylate

2,4-Dibromothiazole (CAS-number 4175-77-3, 150 mg) was added to a solution of tert-butyl 4-{[({[2-(4,4,5,5-tetrunethyl-1,3,2-dioxaborolan-2-yl)phenyl]amino}carbonyl)oxy]methyl}piperidine-1-carboxylate (251 mg) in dry dimethoxyethane (10 ml). A 2 N aqueous solution of sodium carbonate (1.8 ml) was added and a flow of nitrogen was bubbled through the reaction mixture for 15 mins. Tetrakis(triphenylphosphino)palladium (0) (143 mg) was Added and the resulting reaction mixture was heated at 88° C. for 16 h. After cooling the reaction was partitioned between ethyl acetate (150 ml) and water (30 ml). The organic layer was separated, dried (Na₂SO₄) and the solvent was removed to give an oil which was purified by column chromatography on silica. Elution with cyclohexane/ethyl acetate (9:1) gave the title compound as a colorless oil (101 mg).

LC/MS ESI R_{T} 4.25 mins MH⁺ 496, M+2H⁺ 498

### Chloro Intermediate

### tert-Butyl 4-{[({[2-(4-chloro-1,3-thiazol-2-yl)phenyl]amino}carbonyl)oxy]methyl}piperidi&e-1-carboxylate

2,4-Dichlorothiazole (CAS-number 4175-76-2, 114 mg) was added to a solution of tert-butyl 4-{[({[2-(4,4,5,5-tetramethyl-1,3,2-dioxaborolan-2-yl)phenyl]amino}carbonyl)oxy]methyl}piperidine-1-carboxylate (300 mg) in dry dimethoxyethane (12 ml). A 2 N aqueous solution of sodium carbonate (2.2 ml) was added and a flow of nitrogen was bubbled through the reaction mixture for 15 mins. Tetralkis(triphenylphosphino)palladium (0) (170 mg) was added and the resulting reaction mixture was heated at 88° C. for 16 h. After cooling the reaction was partitioned between ethyl acetate (150 ml) and water (50 ml). The organic layer was separated, dried (MgSO₄) and the solvent was removed to give an oil which was purified by column chromatography on silica Elution with cyclohexane/ethyl acetate (4:1) gave the title compound as a colorless oil (150 mg).

LC/MS ESI R_{T} 4.16 mins MH⁺ 452.

### Intermediate 125

### Benzyl 4-fluoro-4-[2-(}[2-(4-methyI-1,3-thiazol-2-yl)pbenyl]amino}oxy)-2-oxoethyl]piperidine-1-carboxylate

Triphosgene (39 mg) was added in one portion to a solutionof 1-Piperidinecarboxylic acid, 4-fluoro-4-(hydroxymethyl)-, phenylmethyl ester (CAS 240400-84-4) (70 mg) and diisopropylethylamine (68 mg) in dry THF (5 ml) at 0° C. under nitrogen. The mixture was allowed to warm to room temperature and stirred for 1 h. 2-(4-methyl-1,3-thiazol-2-yl)aniline (50 mg) was added in one portion and the mixture sty for 16 h, then partitioned between water (10 ml) and ethyl acetate (3×10 ml). The combined organic extracts were washed with brine (10 ml) and dried (MgSO₄). The solvent was evaporated and the residue purified by chromatography on silica Elution with hexane/ethyl acetate 3:1 gave the title compound as a colourless oil (101 mg)

Tlc SiO₂ (Hexane/ethyl acetate 3:1) R_{f} 0.2.

### Intermediate 126

### tert-Butyl (2R,6R)-2,6-dimethyl-4-{[({[2-(4-methyl-1,3-thiazol-2-yl)phenyl]amino}carbonyl)oxy]methyl}pipeiridine-1-carboxylate isomer 1(A) and tert-butyl (2S,6S)-2,6-dimethyl-4-{[({[2-(4-methyl-1,3-thiazol-2-yl)phenyl]amino}carbonyl)oxy]methyl}piperidine-1-carboxylate isomer 2 (B)

Triphosgene (117 mg) was added to a solution of tert-butyl (2R,6R)-4-(hydroxymethyl)-2,6-dimethylpiperidine-1-carboxylate (192 mg) and diisopropylethyiamine (0.27 ml) in dry THF (4 ml) at room temperature under nitrogen. The mixture was stirred for 2 h, then a solution of 2-(4-methyl-1,3-thiazol-2-yl)aniline (150 mg) in dry THF (1 ml) was added dropwise and the mixture stirred for 16 h. The reaction mixture was partitioned between water (15 ml) and ethyl acetate (3×15 ml) and the combined organic extracts washed with brine (20 ml) and dried (MgSO4). The solvent was evaporated and the residue purified by chromatography on silica. Elution with hexane/ethyl acetate 8:1 gave a mixture of the title compounds as a colourless solid (205 mg)

LC/MS ESI R_{T} 4.55 mins MH⁺ 460

The racemate was separated on chiralcel OD 15 ml/min, wavelength 215 nm (2% ethanol/heptane) gave the title compound (A) as a colourless solid (60 mg)

5048-Sample resolved on CHIRALCEL OD-H

Manufacturer DIACEL CHEMICAL INDUSTRIES LTD

Column size 0.46 cm I.D.×25 cm

Coluimn no. ODHOCE-IF029

Eluent 2% Ethanol/Heptane

Plowrate 1 ml/in

Temp. RT

Wavelength 215 nm

Injection volue 15 ul

Retension time 30.97 mins

And the tide compound (B) as a colourless solid (50 mg)

Retension time 35.23 mins

### Intermediate 128

### tert-Butyl 4-{[({[5-fluoro-2-(4-methyl-1,3-thiazol-2-yl)phenyl]amino}carbonyl)oxy]methyl}piperidine-1-carboxylate

A solution of tert-butyl 4-(hydroxymethyl)piperidine-1-carboxylate (CAS-number 123855-51-6; 614 mg) and N,N-diisopropylethylamine in dry THF (5 ml) was added dropwise under nitrogen to a stirred solution of triphosgene (280 mg) in dry THF (5 ml) at 0°. After 2 h at 0°, this mixture was added to a stird solution of 5-fluoro-2-(4-methyl-1,3-thiazol-2-yl)aniline (594 mg) and N,N-diisopropylethylamine (522 μl) in THF (5 ml) under nitrogen. The mixture was stirred at 0° for 3 h, and then at 23° for 16 h. The mixture was evaporated, treated with aqueous saturated sodium bicarbonate (30 ml), and extracted with ethyl acetate (6×80 ml). The combined, dried (Na₂SO₄) organic extracts were evaporated. The residue was adsorbed from warm THF (40 ml) onto silica gel, and this applied to a Biotage Flash™, silica column (40 g). Gradient elution with ethyl acetateyclohexane (4:96 to 14:86 ) afforded the title compound as white crystals.

LC/MS ESI R_{T} 4.41 mins, MH⁺ 450.

### Intermediate 129

### tert-Butyl 4-({[({[2-(4-ethyl-1,3-thiazol-2-yl)-4-fluoro]phenyl}amino)carbonyl]oxy}methyl)-piperidine-1-carboxylate

To a solution of triphosgene (75 m) in dry THF (2.5 ml) at 0° C., diisopropylethylenediamine (132 μl) and tert-butyl-4-(hydroxymethyl)piperidine-1-carboxylate (165 mg) were added and the reaction mixture was stired at 0° C. for 1.5 hours. A solution of 2-(4-ethyl-1,3-thiazol-2-yl)-4-fluoroaniline (168 mg) in dry THF (2.5 ml) and diisopropylethylenediamine (132 μl) were added and the reaction mixture was stirred for 17 hours at room temperature. The reaction mixture was diluted with ethyl acetate (50 ml) and washed with saturated aqueous sodium bicarbonate (50 ml). The aqueous layer was separated and extracted with a further portion of ethyl acetate (50 ml). The organics were combined, dried over MgSO₄ and evaporated to leave a crude yellow solid which was purified by flash column chromatography using a 4:1 hexane/ethyl acetate eluent. After evaporation the title compound was obtained as a yellow solid (321 mg).

LC/MS ESI R_{T} 4.49 mins MH⁺ 464

Tlc SiO₂ (Ethyl acetate/hexane 1:1) R_{f} 0.51

### Intermediate 130

### tert-Butyl 4-({[({[2-(4ethyl-1,3-thiazol-2-yl)-4-hydroxy]phenyl}amino)carbonyl]oxy}methyl)-piperidine-1-carboxylate

To a solution of triphosgene (76 mg) in dry THF (2.5 ml) at 0° C., diisopropylethylenediamnine (134 μl) and tert-butyl-4-(hydroxymethyl)piperidine-1-carboxylate (165 mg) were added and the reaction mixture was stirred at 0° C. for 1.5 hours. A solution of 2-(4-ethyl-1,3-thiazol-2-yl)-4-hydroxyaniline (170 mg) in dry THF (2.5 ml) and diisopropylethylenediamine (134 μl) were then added and reaction mixture was stirred for 17 hours at room temperature. The reaction mixture was diluted with ethyl acetate (50 ml) and washed with saturated aqueous sodium bicarbonate (50 ml). The aqueous layer was separated and extracted with ethyl acetate. The organics were combined, dried over MgSO₄, filtered and evaporated onto silica gel. The crude material was purified by flash column chromatography (dry loading) using a 4:1 hexanelethyl acetate eluent. The title compound was obtained as a white solid (250 mg).

LC/MS ESI R_{T} 4.33 mins MH⁺ 462

Tlc SiO₂ (Ethyl acetatelhexane 1:1) R_{f} 0.49

### Example 1

### Piperidin-4-ylmethyl 2-(1,3-thiazol-2-yl)phenylcarbamate trifluoroacetate

Trifluoroacetic acid (0.2 ml) was added to a solution of tert-butyl 4-{[({[2-(1,3-thiazol-2-yl)phenyl]amino}carbonyl)oxy]methyl}piperidine-1-carboxylate (117 mg) in dichloromethane (2 ml) and the resulting solution was stirred at room temperature for 3.5 h. The solvent was removed to give a yellow oil which was applied to a HPLC autoprep system and eluted with 30% to 60% acetonitrile/water. This gave the title compound as a white solid (30 mg)

LC/MS ESI R_{T} 2.40 mins MH⁺ 318

NMR (CDCl₃ 400MHz; δ) 11.8 (1H, br s, NH) 9.37 (1H, br s, NH) 8.85 (1H, brs, NH) 8.43 (1H, brd, CH) 7.90 (1H, d, CH) 7.78 (1H, dd, CH) 7.41 (1H, ddd, CH) 7.33 (1H, d, CH) 7.09 (1H, ddd, CH) 4.10 (2H, d, CH₂) 3.48 (2H, brd, CH₂eq) 2.92 (2H, br m CH₂ax) 2.12-1.98 (3H, m+brd, CH₂+CH₂eq) 1.67 (2H, brm, CH₂ ax)

### Example 2

### Piperidin-4-ylmethyl 2-(4-methyl-1,3-thiazol-2-yl)phenylcarbamate hydrochloride

A solution of tert-butyl 4({[({2-[4methyl-1,3-thiazol-2-yl]phenyl}amino)carbonyl]oxy}methyl) piperidine-1-carboxylate (165 mg) in dry dichloromethane (8 ml) and trifluoroacetic acid (0.5 ml) was stirred at room temperature for 4 hr under nitrogen. Basified with 8% sodium bicarbonate solution and extracted 3× dichloromethane. The combined organic extracts were dried over MgSO₄. The solvent was evaporated and the residue purified by chromatography (Varian Mega Bond Elut®, Si, 5 g). Elution with methanol/dichloromethane/ammonia (90:10:1) gave a residue which was dissolved in methanol/dichloromethane (1:10) mixture and treated with 1N HCl in ether (0.5 ml). Evaporation of the solvent gave the title compound as a white solid (80 mg).

LC/MS ESI R_{T} 2.72 mins MH⁺ 332

NMR (DMSO 400 MHz; δ) 11.8 (1H, br.s.NH) 8.43 (1H, br.d, CH) 7.73 (1H, dd, CH) 7.38 (1H, ddd, CH) 7.04 (1H, ddd, CH) 6.87 (1H, s, CH) 4.05 (2H, d, CH₂) 3.13 (2H, dt, 2×CHeq.) 2.64 (2H, ddd, 2×CHax.) 2.51 (3H, s, CH₃) 1.88 (1H, m, CH) 1.78 (2H, br.d, 2×CHeq.) 1.25 (2H, dq, 2×CHax.)

### Example 3

### Piperidin-4-ylmethyl 2-(4-ethyl-1,3-thiazol-2-yl)phenylcarbamate Hydrochloride

A solution of tert-butyl 4-{[({[2-(4-ethyl-1,3-thiazol-2-yl)phenyl]amino}carbonyl)oxy]methyl} piperidine-1-carboxylate (78 mg) in methanol (0.5 ml) and dichloromethane (4 ml) was stirred with 1N HCI in ether (1 ml) at room temperature for 16 hr under nitrogen. Evaporation of the solvent, trituation with ether and filtration gave the title compound as a cream solid (54 mg).

LC/MS ESI R_{T} 2.59 MH+ 346

NMR (DMSO 400 MHz; δ) 8.28 (1H, br.d, CH) 7.87 (1H, dd, CH) 7.50-7.45 (2H, ddd+s, 2×CH) 7.18 (1H, ddd, CH) 4.03 (2H, d, CH₂) 3.28 (2H, br.d, 2×CHeq.) 2.93-2.78 (4H, br.t+q, 2×CHax.+CH₂) 1.98 (1H, m, CH) 1.85 (2H, br.d, 2×CHeq.) 1.43 (2H, dq, 2×CHax.) 1.33 (3H, t, CH₃)

### Example 4

### Piperidin-4-ylmethyl 2-(4-propyl-1,3-thiazol-2-yl)phenylcarbamate hydrochloride

A solution of tert-butyl 4-{[({[2-(4-propyl-1,3-thiazol-2-yl)phenyl]amino}carbonyl)oxy]methyl}piperidine-1-carboxylatre (442 mg) in ethyl acetate (10 ml) and methanol (2 ml) was treated with 1.0M ethereal hydrogen chloride (6.5 ml) at 0° C. The mixture was then stirred for 16 h at room temperature. The solvents were evaporated and the residue was triturated in ethyl acetate/ether, to give the title compound as a pale yellow powder (352 mg).

NMR (DMSO 400 MHz; δ) 11.9 (1H, br, s, NH), 9.05 (1H, br s, NH), 8.70 (1H, br s, NH), 8.26 (1H, br d, CH), 7.86 (1H, dd, CH), 7.50-7.45 (2H, ddd+s, 2×CH), 7.16 (1H, ddd, CH), 4.05 (2H, d, CH₂), 3.27 (2H, br d, CH₂ EQ), 2.88 (2H, br t, CH₂ AX), 2.78 (1H, m, CH), 1.88-1.73 (4H, br d+m, 2 x CH₂), 1.45 (2H, m CH₂), 0.96 (3H, t, CH₃)

LC/MS ESI R_{T} 2.77 mins MH⁺ 360

Tlc SiO₂ (Dichloromethane/Methanol/Ammonia 20:2:1) R_{f} 0.4

### Example 5

Piperidin-4-ylmethyl 2-(4-isopropyl-1,3-thiazol-2-yl)phenylcarbamate hydrochloride

A solution of tert-butyl 4-{[({[2-(4-isopropyl-1,3-thiazol-2-yl)phenyl]amino}carbonyl)oxy]methyl} piperidine-1-carboxylate (57 mg) in methanol (1 ml) and dichloromethane (5 ml) was Stirred with 1N HCl in ether (1 ml) at room temperature for 16 hr under nitrogen. Evaporation of the solvent, trituation with ether and filtration gave the title compound as a cream solid (41 mg).

LC/MS ESI R_{T} 2.82 nins MH⁺ 360

NMR (DMSO 400 MHz; δ) 12.0 (1H, br.s, NH) 8.90, 8.55 (2H, 2×v.br.s, NH⁺ ₂) 8.28 (1H, br.d, CH) 7.86(1H, dd, CH) 7.48,7.45 (2H, ddd+s, 2×CH) 7.16 (1H, ddd, CH 4.04 (2H, d, CH₂) 3.28 (2H, br.d, 2×CHeq) 3.12 (1H, m, CH) 2.88 (2H, br.t, 2×CHax) 1.97 (1H, m, CH) 1.34 (6H, d, 2×CH₃)

### Example 6

### Piperidin-4-ylmethyl 2-[4-(cyclopronyl-1,3-thiazol-2-yl]phenylcarbamate hydrochloride

A solution of tert-butyl 4-({[({2-[4-cyclopropyl-1,3-thiazol-2-yl]phenyl}amino)carbonyl]oxy}methyl) piperidine-1-carboxylate (345 mg) in dry dichloromethane (11 ml) and methanol (1 ml) was stirred with 1N HCl in ether (2 ml) at room temperature for 16 hr under nitrogen. Evaporation of the solvent, trituation with ether and filtration gave the title compound as a yellow solid (254 mg).

LC/MS ESI R_{T} 2.78 mins MH⁺ 358.

NMR (DMSO 400 MHz; δ) 8.25 (1H, br.d, CH) 7.83 (1H, dd, CH) 7.50-7.43 (2H, s+ddd, 2×CH) 7.15 (1H, ddd, CH) 4.04 (2H, d, CH₂) 3.30 (2H, br.d, 2×CHeq.) 2.89 (2H, ddd, 2×CHax.) 2.18 (1H, m, CH) 1.98 (1H, m, CH) 1.88 (2H, br.d, 2×CHeq.) 1.43 (2H, br.q.2×CHax.) 1.05-0.92 (4H, 2×m,2×CH₂)

### Example 7

### Piperidin-4-ylmethyl 2-(4-butyl-1,3-thiazol-2-yl)phenylcarbamate hydrochloride

A solution of tert-butyl 4-{[({[2-(4-butyl-1,3-thiazol-2-yl) phenyl]amino}carbonyl)oxy]methyl}piperidine-1-carboxylate (660 mg) in ethyl acetate (10 ml) was treated with 1M ethereal hydrogen chloride (3 ml) . The reaction mixture was stirred at room temperature for 16 h. More ethereal hydrogen chloride (6 ml) was added and the mixture was stirred for a hilrter 16 h. The mixture was then concentrated and the resultant residue was triturated in 5:1, ether/ethyl acetate to give the title compound as a yellow powder (443 mg)

NMR (DMSO 400 MHz; δ) 11.9 (1H, br s, NH), 9.01 (1H, vbr s, NH), 8.67 (1H, br s, NH), 8.29 (1H, br d, CH), 7.86 (1H, dd, CH), 7.50-7.45 (2H, ddd+s, 2×CH), 7.16 (1H, ddd, CH), 4.03 (2H, d, CH₂), 3.27 (2H, br d, CH₂ EQ), 2.88 (2H, m, CH₂, AX), 2.80 (2H, t, CH₂), 2.00 (1H, m, CH), 1.85 (2H, br d, CH₂ EQ), 1.75 (2H, m, CH₂), 1.45 (2H, m, CH₂ AX), 1.38 (2H, m, CH₂), 0.93 (3H, t, CH₃)

Tlc SiO₂ (Dichloromethane/methano/ammonia, 20:2:1) R_{f} 0.44

### Example 8

### Piperidin-4-ylmethyl 2-(4-tert-butyl-1,3-thiazol-2-yl)phenylcarbamate hydrochloride

A solution of tert-butyl 4-{[({[2-(4-tert-butyl-1,3-thiazol-2-yl)phenyl]amino}carbonyl)oxy]methyl}piperidine-1-carboxylate (70 mg) in methanol (0.5 ml) and dichloromethane (5 ml) was stirred with 1N HCl in ether (1 ml) at room temperature for 16 hr under nitrogen. Evaporation of the solvent, trituation with ether and filtration gave the title compound as a yellow solid (43 mg).

LC/MS ESI R_{T} 2.77 mins MH⁺ 374

NMR (DMSO 400 MHz; δ) 12.1 (1H, br.s, NH) 8.30 (1H, br.d, CH) 7.86 (1H, dd, CH) 7.50-7.45 (2H, ddd+s, 2×CH) 7.16 (1H, ddd, CH) 4.03 (2H, d, CH₂) 3.28 (2H, br.d, 2×CHeq.) 2.88 (2H, br.t, 2×CHax.) 1.95 (1H, m, CH) 1.85 (2H, br.d, 2×CHeq.) 1.48-1.35 (11H, m+s, 2×CHax.)

### Example 9

### Piperidin-4-ylmethyl 2-(4-cyclobutyl-1,3-thiazol-2-yl)phenylcarbamate trifluoroacetate

Trifluoroacetic acid (0.3 ml) was added to a solution of tert-butyl 4-{[({[2-(4-cyclobutyl-1,3-thiazol-2-yl)phenyl]amino}carbonyl)oxy]methyl}piperidine-1-carboxylate (285 mg) in dichloromethane (3 ml) and the resulting solution was stirred at room temperature for 2.5 h. The solvent was removed and the residue was dried under vacuum overnight to give the title compound as a yellow solid (274 mg).

LC/MS ESI R_{T} 2.78 mins MH⁺ 372

NMR (CDCl₃ 400 MHz; δ)—12.1 (1H, br s, NH) 8.51 (1H, brs, NH) 8.36 (1H, brd, CH) 7.74 (1H, dd, CH) 7.41 (1H, ddd, CH) 7.09 (1H, ddd, CH) 6.88 (1H, s, CH) 4.11 (2H, d, CH₂) 3.69 (1H, m, CH) 3.54 (2H, br d, CH₂ eq) 2.99 (1H, br m CH₂ax) 2.45-2.32 (1H, m, 2×CH₂) 2.15-2.02 (4H, m, CH₂+CH₂eq) 1.97 (1H, m, CH) 1.61 (2H, brm, CH₂ ax)

### Example 10

### Piperidin-4-ylmethyl 2-(4-pentyl-1,3-thiazol-2-yl)phenylcarbamate (A); and Piperidin-4-ylmethyl 2-(5-butyl-4-methyl-1,3-thiazol-2-yl)phenvlcarbamate(B)

To a solution of tert-butyl 4-{[({[2-(4-pentyl-1,3-thiazol-2-yl)phenyl]ammo}carbonyl)oxy]methyl}piperidine-1-carboxylate (1.1 g) in dichloromethane (15 ml) was added 1 M ethereal hydrogen chloride (4 ml) at 0° C. The mixture was st at room temperature for 16 h. More ethereal hydrogen chloride (7 ml) was added and the mixture stired for a firter 16 h. The solvent was evaporated and the resultant residue was purified by flash column chromatography on silica), eluting with 50:2:1 dichloromethane:methanol:ammonia solution followed by purification by mass directed HPLC to give the title compound (A) (168 mg) as a white powder

LC/MS ESI R_{T} 3.81 mins MH⁺ 387

NMR (CDCl₃ 400 MHz; δ) 12.0 (1H, br s, NH), 8.50 (1H, br s, NH), 8.42 (1H, br d, CH), 7.74 (1H, br d, CH), 7.40 (1H, br t, CH), 7.08 (1H, br t, CH), 6.88 (1H, s, CH), 7-4.5 (1H, v br s, NH), 4.10 (2H, d, CH₂), 3.42 (2H, br d, CH₂ EQ), 2.91-2.78 (4H, m, CH₂AX+CH₂), 2.10-1.94 (3H, br d+m, CH₂ EQ+CH), 1.85-1.75 (2H, m CH₂), 1.63 (2H, m, CH₂ AX), 1.40-1.30 (4H, m, 2×CH₂), 0.91 (3H, t, CH₃)

and the title compound (B) (53 mg) as a pale yellow gum.

LC/MS ESI R_{T} 4.18 mins MH⁺ 387

NMR (CDCl₃ 400 MHz; δ) 12.0 (1H, br s, NH), 8.48 (1H, br S, NH), 9-6 (1H, v br s, NH), 8.38 (1H, br d, CH), 7.65 (1H, br d, CH), 7.35 (1H, br t, CH), 7.03 (1H, br t, CH), 4.10 (2H, d, CH₂), 3.44 (2H, br d, CH₂ EQ), 2.88 (2H, br t, CH2 AX), 2.75 (2H, t, CH₂), 2.38 (3H, s, CH₃), 2.1-1.93 (3H, br d+m, CH₂ EQ+CH), 1.70-1.58 (4H, m, CH₂ AX+CH₂), 1.40 (2H, m, CH₂), 0.95 (3H, t, CH₃)

### Example 11

### Piperidin-4-ylmethyl 2-(4-isobutyl-1,3-thiazol-2-yl)phenylcarbamate

To a solution of tert-butyl 4-{[({[2-(4-isobutyl-1,3-thiazol-2-yl)phenyl]amino}carbonyl)oxy]methyl}piperidine-1-carboxylate (148 mg) in dry dichloromethane (3 ml) was added hydrogen chloride (1M in diethyl ether; 1.5 ml). Reaction was stirred for 2 h at room temperature under nitrogen, then methanol (0.5 ml) was added to aid solubility. Hydrogen chloride (1M in diethyl ether; 1 ml) was added and mixture was stirred at room temperature for 16 h, poured onto saturated aqueous sodium bicarbonate solution and extracted with ethyl acetate (×2). Combined organics were washed with brine, dried over anhydrous magnesium sulphate, filtered and evaporated in vacuo. Residue purified by Varian Mega Bond Elut® (Si, 1 g); elution with 0-50% ethyl acetate in cyclohexane, dichloromethane and fmally dichloromethaime: methanol: ammonia solution (9:1:0.1) gave the title compound as a white solid (79 mg).

LC/MS ESI R_{T} 3.13 mins MH⁺ 374

NMR (CDCl₃ 400 MHz; δ) 11.95 (1H, br s, NH) 8.44 (1H, br d, CH) 7.72 (1H, dd, CH) 7.38 (1H, ddd, CH) 7.05 (1H, ddd, CH) 6.85 (1H, s, CH) 4.02 (2H, d, CH₂) 3.15 (2H, br d, CH₂ eq.) 2.70-2.60 (4H, d+ddd, CH₂+CH₂ ax.) 2.20 (1H, m, CH) 1.95-1.75 (4H+H₂O, br s+m+br d, NH+CH+CH₂ eq.) 1.28 (2H, dq, CH₂ ax.) 0.97 (6H, d, 2×CH₃)

### Example 12

### Piperidin-4-ylmethyl 2-[4-(cyclopropylmethyl)-1,3-thiazol-2-yl]phenylcarbamate

To a solution of tert-butyl 4-({[({2-[4-(cyclopropylmethyl)-1,3-thiazol-2-yl]phenyl}amino)carbonyl]oxy}methyl)piperidine-1-carboxylate (80 mg) in dry dichloromethane (5 ml) was added hydrogen chloride (1M in diethyl ether; 1 ml). Reaction was stirred for 30 mins at room temperature under nitrogen, then dry methanol (0.5 ml) was added to aid solubility and mixture was stirred for a firther 2.5 h. Hydrogen chloride (1M in diethyl ether; 1 ml) was added and reaction stirred at room temperature for 18 h. Reaction poured onto saturated aqueous sodium bicarbonate solution and extracted with ethyl acetate (×2). Combined organics were washed with brine, dried over anhydrous magnesium sulphate, filtered and evaporated in vacuo. Residue purified by Varian Mega Bond Elut® (Si, 1 g); elution with 0-30% ethyl acetate in cyclohexane, dichloromethane and finally dichloromethane/methanol/ammonia solution (9:1:0.1) gave the tide compound as a white residue (57 mg).

LC/MS ESI R_{T} 2.69 mins MH⁺ 372

NMR (CDCl₃ 400 MHz; δ) 11.95 (1H, br s, NH) 8.45 (1H, dd, CH) 7.73 (1H, dd, CH) 7.39 (1H, ddd, CH) 7.05 (1H, ddd, CH) 6.95 (1H, s, CH) 4.04 (2H, d, CH₂) 3.18 (2H, br d, CH₂ eq.) 2.73 (2H, d, CH₂) 2.65 (2H, ddd, CH₂ ax.) 1.88 (1H, m, CH) 1.81 (2H, br d, CH₂ eq.) 1.30 (2H, dq, CH₂ eq.) 1.15 (1H, m, CH) 0.58 (2H, m, CH₂) 0.30 (2H, m, CH₂)

### Example 13

### Piperidin-4-ylmethyl 2-(4-cylopentyl-1,3-thiazol-2-yl)phenylcarbamate trifluoroacetate

Trifuoroacetic acid (0.5 ml) was added to a solution of tert-butyl 4-{[({[2-(4-cyclopentyl-1,3-thiazol-2-yl)phenyl]amino}carbonyl)oxy]methyl}piperidine-1-carboxylate (26 mg) in dichloromethane (5 ml) and the resulting solution was stired at room temperature for 2.5 h. The solvent was removed and the residue was co-evaporated with toluene and methanol to give the title compound as a pale yellow solid (25 mg).

LC/MS ESI R_{T} 2.86 mins MH⁺ 386

NMR (d⁶-DMSO 400 MHz; δ) 11.9 (1H, br s, NH) 8.55 (1H, br s, NH) 8.29 (1H, brd, CH) 8.23 (1H, brs, NH) 7.68 (1H, dd, CH) 7.50-7.45 (2H, ddd+s, 2×CH) 7.16 (1H, ddd, CH) 4.04 (2H, d, CH₂) 3.35-3.22 (3H, m, CH₂+CH-signals obscured by water) 2.90 (2H, br m, CH₂ ax) 2.11 (1H, m, CH₂) 1.95 (1H, m, CH) 1.85 (2H, br d, CH_{2eq}) 1.81-1.63 (6H, m, CH₂rest) 1.39δ (2H, brm, CH₂ax)

### Example 14

### Piperidin-4-ylmethyl 2-(4-cyclohexyl-1,3-thiazol-2-yl)phenylcarbamate trifluoroacetate

Trifluoroacetic acid (0.5 ml) was added to a solution of tert-butyl 4-{[({[2-(4-cyclohexyl-1,3-thiazol-2-yl)phenyl]amino}carbonyl)oxy]methyl}piperidine-1-carboxylate (63 mg) in dichloromethane (5 ml) and the resulting solution was stinred at room temperature for 2.5 h. The solvent was removed and the residue was co-evaporated with toluene and methanol to give the title compound as a pale yellow solid (63 mg).

LC/MS ESI R_{T} 2.94 mins MH⁺ 400

NMR (d⁶ DMSO 400 MHz; δ) 12.0 (1H, br s, NH) 8.55 (1H, br m, NH) 8.30 (1H, brd, CH) 8.25 (1H, brs, NH) 7.85 (1H, dd, CH) 7.48 (1H, ddd, CH) 7.42 (1H, s, CH) 7.15 (1H, ddd, CH) 4.04 (2H, d, CH₂) 3.30 (2H, brd, CH₂eq) 2.90 (2H, br m, CH₂ ax) 2.79 (1H, tt, CHax) 2.08 (1H, brd, CH₂eq) 1.97 (1H, m, CH) 1.90-1.70 (5H, m, CH₂eq+0.5 CH₂eq) 1.58-1.35 (6H, m, 3×CH₂ax) 1.22 (1H, qt, 0.5CH₂ax)

### Example 15

### Piperidin-4-ylmethyl 2-(4-phenyl-1,3-thiazol-2-yl)phenylcarbamate hydrochloride

A solution of tert-butyl 4-{[({[2-(4-phenyl-1,3-thiazol-2-yl)phenyl]amino}carbonyl)oxy]methyl} piperidine-1-carboxylate (74 mg) in methanol (0.5 ml) and dichlororaethane (5 ml) was sd with 1N HCl in ether (1 ml) at room temperature for 16 hr under naitrogen. Evaporation of the solvent, trituation with ether and filtration gave the tide compound as a cream solid (54 mg).

LC/MS ESI R_{T} 2.78 mins MH⁺ 394

NMR (DMSO 400 MHz; δ) 8.45-8.40 (2H, brd+s, 2×CH) 8.15 (2H, dd, 2×CH) 8.06 (1H, dd, CH) 7.67-7.59 (3H, m, 3×CH) 7.55 (1H, ddd, CH) 7.31 (1H, ddd, CH) 4.20 (2H, d, CH₂) 3.40 (2H, br.d, 2×CHeq. +H₂O) 3.02 (2H, ddd, 2×CHax.) 2.15 (1H, m, CH) 2.00 (2H, br.d, 2×CHeq.) 1.58 (2H, dq, 2×CHax.)

### Example 16

### Piperidin-4-ylmethyl 2-(4-thien-3-yl-1,3-thiazol-2-yl)phenylcarbamate hydrochloride

A solution of tert-butyl 4-{[({[2-(4-thien-3-yl-1,3-thiazol-2-yl)phenyl]amino}carbonyl)oxy]methyl} piperidine-1-arboxylate (101 mg) in methanol (0.5 ml) and dichloromethane (5 ml) was stirred with 1N HCl in ether (1 ml) at room temperature for 16 hr under nitrogen. Evaporation of the solvent, trituation with ether and fitration gave the title compound as a cream solid (69 mg).

LC/MS ESI R_{T} 2.74 mins MH⁺ 400

NMR (DMSO 400 MHz; δ) 8.07 (1H, brd, CH) 7.90 (1H, s, CH) 7.74 (1H, dd, CH) 7.69 (1H, dd, CH) 7.50 (1H, dd, CH) 7.48 (1H, dd, CH) 7.28 (1H, ddd, CH) 6.97 (1H, ddd, CH) 3.48 (2H, d, CH₂) 3.04 (2H, br.d, 2×CHeq.) 2.68 (2H, br, t, 2×CHax.) 1.80 (1H, m, CH) 1.65 (2H, br.d, 2×CHwq.) 1.22 (2H, dq, 2×CHax.)

### Example 17

### 4-[({[(2-{4-[(Drimethtlamino)methyl]-1,3-thiazol-2-yl}phenyl)amio]carbonyl}oxy)methyl]piperidine trifluoroacetate

tert-Butyl 4-[({[(2-{4-[(dimethylamino)methyl]-1,3-thiazol-2-yl}phenyl)amino]carbonyl}oxy)methyl]piperidine-1-carboxylate (30 mg) was dissolved in trifluoroacetic acid (1 ml) and water (0.1 ml) added. The solution was stirred at 20° C. for 2.5 hours before evaporating and drying overnight in vacuo to yield the title compound (28.9 mg). NMR (CDCl₃, 400 MHz, δ) 12.55 (1H, br s, NH⁺) 11.3 (1H, s, NH) 9.50 (1H, br d, NH⁺) 8.90 (1H, br d, NH⁺) 8.45 (1H, br d, aromatic CH) 7.76 (1H, dd, aromatic CH) 7.69 (1H, s, aromatic CH) 7.47 (1H, dt, aromatic CH) 7.12 (1H, dt, aromatic CH) 4.48 (2H, s, CH₂) 4.16 (2H, d, CH₂) 3.51 (2H, br d, CH₂) 2.98 (2H, br d, CH₂) 2.91 (6H, s, 2CH₃) 1.99 (1H, m, CH) 1.94-1.75 (4H, m, 2CH₂).

### Example 18

### Piperidin-4-ylmethyl 2-[4-(hydroxymethyl)-1,3-thiazol-2-yl]phenylcarbamate hydrochloride

tert-Butyl 4-({[({2-[4hydroxymethyl)-1,3-thiazol-2-yl]phenyl}amino)carbonyl]oxy}methyl)piperidine-1-carboxylate (84 mg) was suspended in hydrochloric acid (1M in diethyl ether (5 ml) and strred for 4 hours before evaporating to dryness to yield the title compound (61 mg).

NMR (D₂O 400 Mz, δ) 7.77 (1H, dd, aromatic CH) 7.64 (1H, br d, aromatic CH) 7.47 (1H, s, aromatic CH) 7.46 (1H, dt, aromatic CH) 7.29 (1H, br t, aromatic CH) 4.68 (2H, s, CH₂) 3.94 (2H, d, CH₂) 3.38 (2H, br d, CH₂) 2.93 (2H, br t, CH₂) 1.90 (1H, m, CH) 1.86 (2H, br d, CH₂) 1.40 (2H, br q, CH₂)

### Example 19

### Piperidin-4-ylmethyl 2-[4-(metboxymethyl)-1,3-thiazol-2-yl]phenylcarbamate trifluoroacetate

Tert-butyl 4-({[({2-[4-(methoxymethyl)-1,3-thiazol-2-yl]phenyl}amino)carbonyl]oxy}methyl)piperidine-1-carboxylate (8.5 mg) was dissolved in trifluoroacetic acid (1 ml) and water (0.1 ml) added. The solution formed a suspension after 5 minutes, and was stirred for a fiwder 90 minutes at 20° C. before evaporating to dryness to yield the title compound (10.6 mg).

NMR (CDCl₃400 MHz, δ) 11.8 (1H, br s, NH) 9.05 (1H, br s, NH⁺) 8.39 (1H, d, aromatic CH) 8.32 (1H, br s, NH⁺) 7.74 (1H, dd, aromatic CH), 7.41 (dt, aromatic CH) 7.23 (1H, s, aromatic CH)) 7.09 (1H, dt, aromatic CH) 4.64 (2H, s, CH₂) 4.12 (2H, d, CH₂) 3.52 (2H, br d, CH₂) 3.48 (3H, s, CH₃) 2.99 (2H, br q, CH₂) 2.05 (3H, br d, CH₃) 1.69 (2H, br q, CH₂).

### Example 20

### Piperidin-4-ylmethyl 2-{4-[(methylamino)carbonyl]-1,3-thiazol-2-yl}phenylcarbamate trifluoroacetate

Tert-butyl 4-[({[(2-{4-[(methylamino)carbonyl]-1,3-thiazol-2-yl}phenyl)amino]carbonyl}oxy)methyl]piperidine-1-carboxylate (17.8 mg) was dissolved in trifluoroacetic acid (1 ml) and water (0.1 ml) added. The solution was stirred for 1 hr at 20° C. before evaporating and drying in vacuo to yield the title compouind (17.8 mg).

NMR (CDCl₃, 400 MHz, δ) 11.1 (1H, br s, NH) 9.11 (1H, br s, NH⁺) 8.65 (1H, br s, NH⁺) 8.35 (1H, d, aromatic CH) 8.13 (1H, s, aromatic CH) 7.73 (1H, dd, aromatic CH) 7.48 (dt, aromatic CH) 7.18 (1H, br q, NH) 7.13 (1H, dt, aromatic CH) 4.17 (2H, d, CH₂) 3.52 (2H, br d, CH₂) 3.03 (3H, d, CH₃) 2.96 (2H, br q, CH₂) 2.06 (1H, m, CH) 2.00 (2H, br d, CH₂) 1.73 (2H, br q, CH₂)

### Example 21

### Ethyl 2-(2-{[(piperidin-4-yhmethoxy)carbonyl]amino}phenyl)-1,3-thiazole-4-carboxylate trifluoroacetate

Tert-butyl 4-({[({2-[(ethoxycarbonyl)-1,3-thiazol-2-yl]phenyl}amino)carbonyl]oxy}methyl)piperidine-1-carboxylate (50 mg) was dissolved in tfluoroacetic acid (1 ml) and water (0.1 ml) added. The solution was stirred for 1 hr at 20° C. before evaporating and drying in vacuo to yield the title compound (56.8 mg).

NMR (CDCl₃, 400 MHz, δ) 11.9 (1H, br s, NH) 9.25 (1H, br s, NH⁺) 8.42 (1H, d, aromatic CH) 8.14 (1H, s, aromatic CH) 7.76 (1H, dd, aromatic CH) 7.49 (1H, br s, NH) 7.48 (dt, aromatic CH) 7.11 (1H, dt, aromatic CH) 4.39 (2H, q, CH₂) 4.19 (2H, d, CH₂) 3.62 (2H, br d, CH₂) 3.12 (2H, br q, CH₂) 2.02 (4H, m, 2CH₂) 1.40 (3H, t, CH₃)

### Example 22

### Piperidin-4-ylmethyl 2-{4-[2-(benzyloxy)ethyl]-1,3-thiazol-2-yl}phenylcarbamate trifluoroacetate

To a solution of tert-butyl 4-[({[(2-{4-[2-(benzyloxy)ethyl]-1,3-thiazol-2-yl}phenyl)amino]carbonyl}oxy)methyl]piperidine-1-carboxylate (100 mg) in dichloromethane (1 ml) was added trifluoroacetic acid (0.13 ml). After stirring for 24 hours at room temperature, the solvents were evaporated. The crude residue was triturated with diethyl ether, evaporated and dried under vacuum to give the title compound as brown solid (114 mg).

NIMR (d⁶-DMSO 400 MHz; δ) 11.87 (1H, s, NH) 8.53 (1H, br. s, NH₂⁺) 8.26 (1H, d, aromatic CH) 8.21 (1H, br. s, NH₂⁺) 7.86 (1H, d, aromatic CH) 7.53 (1H, s, thiazole CH) 7.47 (1H, t, aromatic CH) 7.32-7.21 (5H, m, phenyl) 7.17 (1H, t, aromatic CH) 4.51 (2H, s, OCH₂Ar) 4.00 (2H, d, OCH₂piperidine) 3.87 (2H, t, O CH₂) 3.27 (2H, br. d, CH₂N⁺) 3.09 (2H, t, thiazole CH₂) 2.92-2.80 (2H, m, CH₂N⁺) 1.98-1.87 (1H, m, CH₂ of piperidine ring) 1.82 (2H, br. d, CH₂ of piperidine ring) 1.43-1.31 (2H, m, CH₂ of piperidine ring).

LC/MS ESI R_{T} 3.13 mins, MH⁺ 452.6

### Example 23

### Piperidin-4-ylmethyl 2-(4-acetyl-1,3-thiazol-2-yl)phenylcarbamate trifluoroacetate

To a solution tert-butyl 4-{[({[2-(4-acetyl-1,3-thiazol-2-yl)phenyl]amino}carbonyl)oxy]methyl}piperidine-1-carboxylate (19 mg) in dichloromethane (0.5 ml) was added trifluoroacetic acid (0.05 ml). After stirring for 90 mins at room temperature, the solvents were evaporated. The crude oil was triturated with diethyl ether to give, after drying, the title compound as a white solid (21 mg).

NMR (d⁶-DMSO 400 MHz; δ) 11.75 (1H, s, NH) 8.65 (1H, s, thiazole CH) 8.53 (1H, br. s, NH⁺) 8.25 (1H, d, aromatic CH) 7.98 (1H, d, aromatic CH) 7.55 (1H, t, aromatic CH) 4.05 (2H, d, OCH₂) 3.30 (2H, m, CH₂N⁺) 2.96-2.83 (2H, m, CH₂N⁺) 2.67 (3H, s, CH₃) 2.02-1.91 (1H, m, CH of piperidine ring) 1.90-1.81 (2H, m, CH₂ of piperidine ring) 1.45-1.32 (2H, m, CH₂ of piperidine ring).

LC/MS ESI R_{T} 2.56 mins, MH⁺ 360.5

### Example 24

### Piperidin-4-ylmethyl 2-[4-(1-hydroxyethyl)-1,3-thiazol-2-yl]phenylcarbamate

To a solution tert-butyl 4-({[({2-[4(1-hydroxyethyl)-1,3-thiazol-2-yl]phenyl}amino)carbonyl]oxy}methyl)piperidine-1-carboxylate (41 mg) in dichloromethane (1 ml) was added trifluoroacetic acid (0.07 ml). After stining for 18 hours at room temperature, the solvents were evaporated. The crude oil was re-dissolved in ethyl acetate, washed with saturated aqueous sodium bicarbonate and water then dried (MgSO₄). The solvent was evaporated to give, after drying the title compound as a pale yellow solid (33 mg).

NMR (d⁶-DMSO 400 MHz; δ) 11.88 (1H, s, NH) 8.24 (1H, d, aromatic CH) 7.87 (1H, d, aromatic CH) 7.54 (1H, s, thiazole CH) 7.46 (1H, t, aromatic CH) 7.17 (1H, t, aromatic CH) 4.88 (1H, q, CHOH) 4.03-3.98 (2H, m, OCH₂) 3.12-3.03 (2H, m, CH₂N) 2.70-2.57 (2H, m, CH₂N) 1.88-1.77 (1H, m, CH of piperidine ring) 1.76-1.67 (2H, m, CH₂ of piperidine zing) 1.48 (3H, d, CH₃) 1.32-1.03 (2H, m CH₂ of piperidine ring).

LC/MS ESI R_{T} 2.54 mins, MH⁺ 362.2

### Example 25

### (R)-Piperidin-4-ylmethyl 2-[4-(1-hydroxyethyl)-1,3-thiazol-2-yl]phenylcarbamate hydrochloride

To a solution (R)-tert-butyl 4-({[({2-[4-(1-hydroxyethyl)-1,3-thiazol-2-yl]phenyl}amino)carbonyl]oxy}methyl)piperidine-1-carboxylate (150 mg) methanol (3 ml) was added a 1M solution of hydrogen chloride in diethyl ether (15 ml). After stirinng for 3 hours at room temperature, the solvents were evaporated to give after drying the title compound as a pale yellow solid (141 mg).

NMR (d⁶-DMSO 400 MHz; δ) 11.79 (1H, s, NH) 8.71 (1H, br. s, NH₂⁺) 8.36 (1H, br. s, NH₂⁺) 8.25 (1H, d, aromatic CH) 7.87 (1H, d, aromatic CH) 7.54 (1H, s, thiazole CH) 7.47 (1H, t, aromatic CH) 7.17 (1H, t, aromatic CH) 5.51 (1H, br. s, OH) 4.89 (1H, q, CHOH) 4.03 (2H, d, OCH₂) 3.32-3.25 (2H, m, CH₂N) 2.94-2.82 (2H, m, CH₂N) 2.00-1.91 (1H, m, CH of piPeridine rig) 1.88-1.82 (2H, m, CH₂ of piperidine ring) 1.50 (3H, d, CH₃) 1.32-1.03 (2H, m, CH₂ of piperidine ring).

LC/MS ESI R_{T} 2.55 mins, MH⁺ 362

### Example 26

Piperidin-4-ylmethyl 2-[4-(2-hydroxyethyl)-1,3-thiazol-2-yl]phenylcarbamate hydrochloride

To solution of tert-Butyl 4-({[({2-[4-(2-hydroxyethyl)-1,3-thiazol-2-yl]phenyl}amino)carbonyl]oxy}methyl)piperidine-1-carboxylate (100 mg) in methanol (1 ml) was added a 1M hydrogen chloride solution in diethyl ether (5 ml). After stirring for 3 hours at room temperature, the solvents were evaporated. The crude oil was triturated with diethyl ether and after drying under vacuum the title compound was obtained as a pale yellow solid (96.2 mg).

NMR (d⁶-DMSO 400 MHz; δ) 11.82 (1H, s, NH) 8.85 (1H, br s, NH₂⁺) 8.48 (1H, br s, NH₂⁺) 8.25 (1H, d, aromatic CH) 7.86 (1H, d, aromatic CH) 7.48 (1H, s, thiazole CH) 7.45 (1H, t, aromatic CH) 7.17 (1H, t, aromatic CH) 4.04 (2H, d, OCH₂) 3.83 (2H, t, CH₂OH) 3.32-3.23 (2H, m, CH₂N) 2.94 (2H, t, CH₂) 2.93-2.82 (2H, m, CH₂N) 2.04-1.92 (1H, m, CH of piperidine ring) 1.90-1.82 (2H, m, CH₂ of piperidine ring) 1.50-1.46 (2H, m, CH₂ of piperidine ring).

LC/MS ESI R_{T} 2.60mins, MH⁺ 362.3

### Example 27

### Piperidin-4-ylmethyl 2-[4-(trifluoromethyl)-1,3-thiazol-2-yl]phenylcarbamate hydrochloride

A solution of tert-butyl 4-({[({2-[4-trifluoromethyl-1,3-thiazol-2-yl]phenyl}amino)carbonyl]oxy}methyl) piperidine-1-carboxylate (60 mg) in dry dichloromethane (3 ml) and methanol (0.5 ml) was stirred with 1N HCl in ether (1 ml) at room temperature for 16 hr under nitrogen. Evaporation of the solvent, trituation with ether and filtration gave the title compound as a yellow solid (38 mg).

LC/MS ESI R_{T} 2.58 mins MH⁺ 386

NMR (DMSO 400 MHz; δ) 8.65 (1H, s, CH) 8.04-7.95 (2H, m, 2×CH) 7.57 (1H, ddd, CH) 7.30 (1H, ddd, CH) 4.00 (2H, d, CH₂) 3.28 (2H, br.d, 2×CHeq.) 2.88 (2H, m, 2×CHax.) 1.95 (1H, m, CH) 1.81 (2H, br.d, 2×CHeq.) 1.38 (2H, br.q.2×CHax.)

### Example 28

### Piperidin-4-ylmethyl 2-[4-(difluoromethyl)-1,3-thiazol-2-yl]phenylcarbamate

To a solution tert-butyl 4-({[({2-[4-(difluoromethyl)-1,3-thiazol-2-yl]phenyl}amino)carbonyl]oxy}methyl)piperidine-1-carboxylate (85 mg) in dichloromethane (1 ml) was added tifuoroacetic acid (0.27 ml). After s&ting for 2 hours 45 mins at room temperature, the solvents were evaporated. The crude oil was re-dissolved in ethyl acetate and washed with 0.5 M aqueous sodium hydroxide then water. The solvent was evaporated to give, after drying the title compound as a pale yellow soUd (63 mg).

NMR (CDCl₃ 400 MHz; δ) 11.78 (1H, s, NH) 8.40 (1H, d, aromatic CH) 7.75 (1H, d, aromatic CH) 7.43 (1H, t, aromatic CH) 7.36 (1H, s, thiazole CID 7.09 (1H, t, aromatic CH) 5.50 (2H, d, CH₂F), 4.11 (2H, d, OCH₂) 3.45 (2H, br. d, CH₂N) 2.92 (2H, br. t, CH₂N), 2.08-1.96 (3H, m, CH and CH₂ of piperidine ring) 1.74-1.61 (2H, m, CH₂ of piperidine ring)

LC/MS ESI R_{T} 2.45 mins, MH⁺ 367.4

### Example 29

### Piperidin-4-ylmethyl 2-[4-(fuoromethyl)-1,3-thiazol-2-yl]phenylcarbamate trifluoroacetate

To a solution tert-butyl 4-({[({2-[4-(fluoromethyl)-1,3-thiazol-2-yl]phenyl}amino)carbonyl]oxy}methyl)piperidine-1-carboxylate (45 mg) in dichioromethane (2 ml) was added truoroacetic acid (0.13 ml). After stining for 3 hours at room temperature, the solvents were evaporated. The crude oil was triturated with diethyl ether to give, after drying, the title compound as a white solid (46 mg).

NMR (d⁶-DMSO 400 MHz; δ) 11.44 (1H, s, NH) 8.53 (1H, br. s, NH₂⁺) 8.22 (1H, s, thiazole CH) 8.20 (1H, br. s, NH₂⁺) 8.08 (1H, d, aromatic CH) 7.94 (1H, d, aromatic CH) 7.50 (1H, t, aromatic CH) 7.23 (1H, t, aromatic CH) 7.16 (1H, t, CF₂H) 4.00 (2H, d, OCH₂) 3.34-3.22 (2H, m, CH₂N⁺) 2.93-2.80 (2H, m, CH₂N⁺) 2.00-1.87 (1H, m, CH of piperidine ring) 1.86-1.77 (2H, m, CH₂ of piperidine ring) 1.42-1.28 (2H, m, CH₂ of piperidine ring).

LC/MS ESI R_{T} 2.43 mins, MH⁺350.4

### Example 30

### Piperidin-4-ylmethyl 2-[4-(1,1-difluoroethyl)-1,3-thiazol-2-yl]phenylcarbamate

To a solution of tert-butyl 4-({[({2-[4(1,1-difluoroethyl)-1,3-thiazol-2-yl]phenyl}amino)carbonyl]oxy}methyl)piperidine-1-carboxylate (40 mg) in dichloromethane (3 ml) was added trifuoroacetic acid (0.21 ml). After sti:mng for 5 hours at room temperature, the solvents were evaporated. The crude residue was re-dissolved in ethyl acetate and washed with 0.5M sodium hydroxide then water. After drying MgSO₄), the solvent was evaporated to give a pale brown residue which was further purify by mass directed preparative HPLC to afford the title compound as white solid (5 mg).

NMR (CDCl₃ 400 MHz; δ) 11.76 (1H, s, NHCO) 8.54 (1H, s, piperidine NH) 8.42 (1H, d, aromatic CH) 7.74 (1H, d, aromatic CH) 7.54 (1H, s, thiazole CH) 7.43 (1H, t, aromatic CH) 7.08 (1H, t, aromatic CH) 4.09 (1H, d, OCH₂) 3.45-3.33 (2H, m, CH₂N) 2.88-2.75 (2H, m, CH₂N) 2.08 (3H, t, CH₃CF₂) 2.00-1.90 (3H, m, CH and CH₂ of piperidine ring) 1.69-1.53 (2H, m, CH₂ of piperidine ring)

LC/MS ESI R_{T} 2.73 mins, MH⁺ 382.5

### Example 31

### Piperidin-4-ylmethyl 2-[4-(2-fluoroethyl)-1,3-thiazol-2-yl]phenylcarbamate

To a solution of tert-butyl 4-({[({2-[4-(2-fluoroethyl)-1,3-thiazol-2-yl]phenyl}amino)carbonyl]oxy}methyl)piperidine-1-carboxylate (55 mg) in dichloromethane (1 ml) was added tifluoroacetic acid (0.05 ml). After stiing for 3 hours at room temperature, the solvents were evaporated. The crude oil was re-dissolved in ethyl acetate and washed with 0.5 M aqueous sodium hydroxide then water. The solvent was evaporated to give, after drying, the title compound as a pale brown solid (35 mg).

NMR (CDCl₃ 400 MHz; δ) 11.89 (1H, s, NH 8.42 (1H, d, aromatic CH) 7.73 (1H, d, aromatic CH) 7.40 (1H, t, aromatic CH) 7.05 (1H, t, aromatic CH) 7.02 (1H, s, thiazole CH) 4.88 (1H, dt, CH₂F) 4.05 (2H, d, OCH₂) 3.24 (2H, dt, thiazol CH₂) 3.21-3.13 (2H, m, CH₂NH) 2.68 (2H, td, CH₂NH) 1.90-1.60 (3H, m, CH₂ and CH of piperidine ring) 1.37-1.25 (2H, m, CH₂ of piperidine ring)

LC/MS ESI R_{T} 2.77 mins, MH⁺ 364.2

### Example 32

### Piperidin-4-ylmethyl 2-[4-(2.2-difiuoroethyl)-1,3-thiazol-2-yl]phenylcarbamate trifluoroacetate

A solution of tert-butyl 4-({[({2-[4-(2,2-difuoroethyl)-1,3-thiazol-2-yl]phenyl}amino)carbonyl]oxy}methyl)piperidine-1-carboxylate (46 mg) and trifluoroacetic acid (0.1 ml) in dichloromethane (3 ml) was stirred at room texaperature for 2 hours 20 minutes. After evaporation of the residue, the residue was triturated with diethyl ether. After drying under vacuum for 12 hours, the title compound was obtained as a pale yellow solid (49 mg).

NMR (CDCl₃ 400 MHz; δ) 11.45 (1H, s, NH) 8.22 (1H, br d, aromatic CH) 7.90 (1H, dd, aromatic CH) 7.70 (1H, s, thiazole CH) 7.49 (1H, d, taromatic CH) 7.19 (1H, dt, aromatic CH) 6.41 (1H, tt, CF₂H) 4.03 (2H, d, OCH₂) 3.46 (2H, dt, CH₂CF₂) 3.32-3.25 (2H, m, CH₂N) 2.89 (2H, bt, CH₂N) 2.01-1.90 (1H, m, piperidine CH) 1.86 (2H, br d, piperidine CH₂) 1.45-1.31 (2H, m, piperidine CH₂).

LC/MS ESI R_{T} 2.88 mins MH⁺ 382.4

### Example 33

### Pipendin-4-ylmethyl 2-(4,5-dimethyl-1,3-thiazol-2-yl)phenylcarbamate hydrochloride

A solution of tert-butyl 4-{[({[2-(4,5-dimethyl-1,3-thiazol-2-yl)phenyl]amino}carbonyl)oxy]methyl} piperidine-1-carboxylate (55 mg) in methanol (0.5 ml) and dichloromethane (5 ml) was stirred with 1N HCl in ether (1 ml) at room temperature for 16 hr under nitrogen. Evaporation of the solvent, trituation with ether and filtration gave the title compound as a yellow solid (42 mg).

LC/MS ESI R_{T} 2.60 mins MH⁺ 346

NMR (DMSO 400 MHz; δ) 8.04 (1H, br.d, CH) 7.56 (1H, dd, CH) 7.23 (1H, ddd, CH) 6.95 (1H, ddd, CH) 3.85 (2H, d, CH₂) 3.09 (2H, br.d, 2×CHeq.) 2.69 (2H, m, 2×CHax.) 2.22 (3H, s, CH₃) 2.18 (3H, s, CH₃) 1.78 (1H, m, CH) 1.65 (2H, br.d, 2×CHeq.) 1.25 (2H, br.q, 2×CHax.)

### Example 34

### Piperidin-4-ylmethyl 2-(5-methyl-1,3-thiazol-2-yl) henvlcarbamate hydrochloride

A solution of tert-butyl 4-{[({[2-(5-methyl-1,3-thiazol-2-yl)phenyl]amino}carbonyl)oxy]methyl} piperidine-1-carboxylate (97 mg) in methanol (1 ml) and dichloromethane (5 ml) was stirred with 1N HCl in ether (1 ml) at room temperature for 16 hr under nitrogen. Evaporation of the solvent, trituation with ether and filtration gave the tide compound as a cream solid (77 mg).

LC/MS ESI R_{T} 2.49 mins MH⁺ 332

NMR (DMSO 400 MHz; δ) 8.26 (1H, br.d, CH) 7.80 (1H, dd, CH) 7.73 (1H, s, CH) 7.46 (1H, ddd, CH) 7.18 (1H, ddd, CH) 4.05 (2H, d, CH₂) 3.28 (2H, br.d, 2×CHeq.) 2.87 (2H, br.m, 2×CHax.) 2.52 (3H, s, CH3 obscured by DMSO) 2.00 (1H, m, CH) 1.83 (2H, br.d, 2×CHeq.) 1.42 (2H, br.q, 2×CHax)

### Example 35

### Piperidin-4-ylmethyl 2-{4-methyl-5-[(methylamino)carbonyl]-1,3-thiazol-2-yl}phenylcarbamate trifluoroacetate

To a solution of tert-butyl 4-[({[(2-{4-methyl-5-[(methylamino)carbonyl]-1,3-thiazol-2-yl}phenyl)amino]carbonyl}oxy)methyl]piperidine-1-carboxylate (50 mg) in dichloromethane (5 ml) was added tuoracetic acid (2 ml). The mixture was stirred at room temperature for 2 h. The solvents were then evaporated to leave the title compound as a pale yellow foam (40 mg).

NMR (CDCl₃ 400 MHz; δ) 11.7 (1H, s, NH) 9.03 (1H, br s, NH) 8.52 (1H, br s, NH), 8.39 (1H, br d, CH), 7.67 (1H, dd, CH), 7.42 (1H, ddd, CH), 7.065 (1H, ddd, CH), [6.95—excess CF₃COOH], 6.07 (1H, br q, NH), 4.11 (2H, d, CH₂), 3.52 (2H, br d, CH₂ EQ), 3.01 (3H, s, CH₃), 3.0-2.90 (2H, br t, CH₂ AX), 2.70 (3H, s, CH₃), 2.10-1.93 (3H, br m, CH+CH₂ EQ), 1.68 (2H, br m, CH₂ AX)

LC/MS ESI R_{T} 2.49 mins MH⁺ 389

Tlc SiO₂ (Dichloromethane/methanol/ammonia, 20:2:1) R_{f} 0.1

### Example 36

### Piperidin-4-ylmethyl 2-(4,5,6,7-tetrahydro[1,3]thiazolo[5,4-c]pyridin-2-yl)phenylcarbamate

To a solution of tert-butyl 2-{2-[({[1-(tert-butoxycarbonyl)piperidin-4-yl]methoxy}carbonyl)amino]phenyl}-6,7-dihydro[1,3]thiazolo[5,4-c]pyridine-5(4H)-carboxylate (27 mg) in dry dichloromethane (2 ml) was added hydrogen chloride (1M in diethyl ether; 1 ml). Reaction was stirred for 1 h at room temperature under nitrogen, then dry methanol (0.5 ml) was added to aid solubility. Hydrogen chloride (1M in diethyl ether; 1 ml) was added and mixture was stirred at room temperature for 18 h. Reaction evaporated in vacuo and residue purified by Varian Mega Bond Elut® (Si, 0.5 g); elution with 0-100% ethyl acetate in cyclohexane, dichloromethane and finally dichloromethane:methanol:ammonia solution (98:2:0.2 to 85:15:1.5) gave the title compound as an off white residue (15 mg).

LC/MS ESI R_{T} 2.16 mins MH⁺ 373

NMR (DMSO 400 MHz; δ) 11.6 (1H, br s, NH) 8.22 (1H, br d, CH) 7.80 (1H, dd, CH) 7.45 (1H, ddd, CH) 7.15 (1H, ddd, CH) 4.10 (1H, br s, NH) 4.00 (2H, d, CH₂) 3.95 (2H, s, CH₂) 3.11 (2H, br dt, CH₂ eq.) 3.03 (2H, t, CH₂) 2.75 (2H, br t, CH₂) 2.68 (2H, ddd, CH₂ ax.) 1.85 (1H, m, CH) 1.72 (2H, br d, CH₂ eq.) 1.26 (2H, dq, CH₂)

### Example 37

Piperidin-4-ylmethyl 2-(5,6-dihydro-4H-cyclopenta[d][1,3]thiazol-2-yl)phenylcarbamate

To a solution of tert-butyl 4-{[({[2-(5,6-dihydro-4H-cyclopenta[d][1,3]thiazol-2-yl)phenyl]amino}carbonyl)oxy]methyl}piperidine-1-carboxylate (162 mg) in dry dichloromethane (3 ml) was added hydrogen chloride (4M in 1,4-dioxane; 1 ml). Reaction was stirred for 15 mins at room temperature under nitrogen, then dry methanol (0.5 ml) was added to aid solubility. The mixture was stirred at room temperature for 18 h, evaporated in vacuo and the residue purified by Varian Mega Bond Elut® (Si, 5 g). Elution with dichloromethane followed by dichloromethane: methanol: ammonia solution (99:1:0.1 to 90:10:1) gave the title compound as a white solid (105 mg).

LC/MS ESI R_{T} 3.00 mins MH⁺ 358

NMR (DMSO 400 MHz; δ) 11.4 (1H, br s, NH) 8.23 (1H, br d, CH) 7.82 (1H, br d, CH) 7.45 (1H, ddd, CH) 7.16 (1H, ddd, CH) 4.02 (2H, d, CH₂) 3.18 (2H, br d, CH₂ eq.) 2.96 (2H, br t, CH₂) 2.88 (2H, br t, CH₂) 2.75 (2H, ddd, CH₂ ax.) 2.50 (2H, m, CH₂ obscured by DMSO) 1.92 (1H, m, CH) 1.77 (2H, br d, CH₂ eq.) 1.33 (2H, dq, CH₂ ax.)

### Example 38

### Piperidin-4-ylmetlyl 2-[4-bromo-1,3-thiazol-2-yl]phenylcarbamate trifluoroacetate

A solution of tert-butyl 4-({[({2-[4-bromo-1,3-thiazol-2-yl]phenyl}amino)carbonyl]oxy}methyl)piperidine-1-carboxylate (101 mg) and trifluoroacetic acid (0.3 ml) in dichloromethane (5 ml) was stirred at room temperature for 6 hours. After evaporation of the solvent, the residue was dryied under vacuum for 12 hours to give the title compound as a pale yellow solid solid (102 mg).

LC/MS ESI R_{T} 2.66 mins M+2H⁺ 398

### Example 39

### Piperidin-4-ylmethyl 2-[4 chloro-1,3-thiazol-2-yl]phenylcarbamate trifluoroacetate

A solution of tert-butyl 4-({[({2-[4-chloro-1,3-thiazol-2-yl]phenyl}amino)carbonyl]oxy}methyl)piperidine-1-carboxylate (150 mg) and trifluoroacetic acid (0.5 ml) in dichloromethane (7.5 ml) was stirred at room temperature for 16 hours. After evaporation of the solvent, the residue was dried under vacuum for 12 hours to give the title compound as a pale yellow solid (156 mg).

LC/MS ESI R_{T} 2.72 mins MH⁺ 352

### Example 41

### Piperidin-4-ylmethyl 5-fluoro-2-(4-methyl-1,3-thiazol-2-yl)phenylcarbamate hadrochloride

A solution of tert-butyl 4-{[({[5-fluoro-2-(4-methyl-1,3-thiazol-2-yl)phenyl]amino}carbonyl)oxy]methyl}piperidine-1-carboxylate (300 mg) in dichloromethane was treated with a solution of 4.0M hydrogen chloride in 1,4-dioxan (2 ml) at 23° and stirred for 1.5 h. The mixture was evaporated to give the title compound as cream crystals (228 mg).

NMR (D₂O 400 MHz; δ) 7.48-7.39 (2H, m, 2×aromatic CH), 6.88 (1H, s, oxazole CH), 6.66 (1H, m, aromatic CH), 3.77 (2H, d, CH₂), 3.21 (2H, m, CH₂), 2.77 (2H, m, CH₂), 2.14 (3H, s, CH₃), 1.83-1.68 (3H, m, CH&CH₂), 1.25 (2H, m, CH₂).

LC/MS ESI R_{T} 2.96 mins, MH⁺ 350.

### Example 42

### Piperidine-4-ylmethyl(2-{4-ethyl-1,3-thiazol-2-yl}4-fluoro)phenylcarbamate trifluoro acetate

To a solution of tert-butyl 4-({[({[2-(4-ethyl-1,3-thiazol-2-yl)-4-fluoro]phenyl}amino)carbonyl]oxy} methyl)-piperidine-1-carboxylate (100 mg) in DCM (5 ml) 10% TFA (331 μl) was added. The reaction mixture was stirred for 5 hours at room temperature then evaporated to give the title compound as a yellow solid (110 mg).

LC/MS ESI R_{T} 3.04 mins MH⁺ 364

NMR (d⁶ DMSO 400 MHz; δ) 10.75 (1H, s, NH) 8.55 (1H, s, NH⁺) 8.18 (1H, s, NH⁺) 8.28 (1H, d, aromatic CH) 7.73 (1H, dd, aromatic CH) 7.50 (1H, s, thiazole CH) 7.32-7.41 (1H, m, aromatic CH) 4.12 (2H, d, OCH₂) 3.26 (2H, d, equatorial CH₂N) 2.80-2.92 (2H, m, axial CH₂N) 2.77-2.80 (2H, m, CH₂) 1.90-2.02 (1H, m, CH of piperidine ring) 1.82 (2H, d, equatorial CH₂ of piperidine ring) 1.30-1.A1 (2H, m, axial CH₂ of piperidine ring) 1.32 (3H, t, CH₃)

### Example 43

### Piperidine-4-ylmethyl(2-{4-ethyl-1,3-thiazol-2-yl}4-hydroxy)phenylcarbamate hydrochloride

To a solution of tert-butyl-4-({[({2-[4-ethyl-1,3-thiazol-2-yl]-4-hydroxy}phenyl)amino]oxy}methyl)piperidine-1-carboxylate (100 mg) in methanol (2.5 ml) 1M HCl in 1,4-dioxane was added (2.5 ml). The reaction mixture was stirred for 1 hour at room temperature then evaporated to give the title compound as a pale yellow solid (81 mg).

LC/MS ESI R_{T} 2.72 mins MH⁺ 362

NMR (d⁶ DMSO 400 MHz; δ) 10.52 (1H, s, NH) 9.84 (1H, s, OH) 8.73 (1H, s, NH⁺) 8.34 (1H, s, NH⁺) 7.88 (1H, s, aromatic CH) 7.41 (1H, s, thiazole CH) 7.28 (1H, s, aromatic CH) 7.88 (1H, d, aromatic CH) 3.96 (2H, d, OCH₂) 3.28 (2H, d, equatorial CH₂N) 2.80-2.92 (2H, m, axial CH₂N) 2.73-2.84 (2H, m, CH₂) 1.94 (1H, s, CH of piperidine ring) 1.82 (2H, d, equatorial CH₂ of piperidine ring) 1.32-1.45 (2H, m, axial CH₂ of piperidine ring) 1.31 (3H, t, CH₃)

### Example 49

### (1-Butylpiperidin-4-yl)methyl 2-(4-methyl-1,3-thiazol-2-yl)phenyl]carbamate hydrochloride

Piperidin-4-ylmethyl 2-(4-methyl-1,3-thiazol-2-yl) phenylcarbamate (200 mg) dissolved in methanol (8 ml) at room temperature. Butyraldehyde (0.18 ml) was added and the solution was stirred for 24 hours at room temperature. A solution of sodium borohydride (25 mg) in water (0.5 ml) was added and stirred for 30 minutes. Further water was added (5 ml) and mixture acidified with 2N Hydrochloric acid to pH1, neutraised with 8% aqueous sodium bicarbonate solution and extracted with ethyl acetate (×3). The combined organic extracts were dried (MgSO₄) and solvent evaporated. The residue purified by column chromatography on silica Elution with dichloromethane/methanol (2%) and salt formation with 1.0 M HCl solution in diethyl ether gave the title compound as a yellow solid (20 mg)

LC/MS ESI R_{T} 2.79 mins MH⁺ 388

NMR (MeOH-d4 400 MHz; δ) 7.95 (1H, d, CH aromatic) 7.7 (1H,d,CH aromatic) 7.4 (1H, t, CH aromatic) 7.3 (1H, s, CH thiazole) 7.15 (1H, t, CH aromatic) 4.0 (2H, d, OCH₂) 3.55 (2H, d, CH₂ piperidine) 3.0 (2H, t, CH₂) 2.9 (2H, t, CH₂ piperidine) 2.45 (3H, s, CH₃)

### Example 50

### 1-{2-[(Methylsulphonyl)amino]ethyl}piperidin-4-yl)methyl 2-(4-methyl-1,3-thiazol-2-yl)phenylcarbamate hydrochloride

Triphosgene (94 mg) dissolved in dry tetrahydrofuran (10 ml) at room temperature and solution stirred under nitrogen. This was cooled to 0° C. and a solution of N-[2-[4-(Hydroxymethyl)-1-piperidinyl]ethyl]sulphonamide (190 mg) with N,N-Diisopropylethylarine (0.14 ml) was added. Stirred for 1 hour at 0° C. A solution of 2-(4-methyl-1,3-thiazol-2-yl)aniline (150 mg) in dry tetrahydrofuran (4 ml) added and allowed the temperature of reaction to reach room temperature. Stirred for 24 hours. Filtered and the filtrate concentrated to a yellow oil. Purified by column chromatography on silica, eluted with dichloromethane/methanol (2%) increasing to dichloromethane/methanol (5%). Salt formation with 1.0 M HCl solution in diethyl ether gave the title compound as a yellow solid (27 mg)

LC/MS ESI R_{T} 2.87 mins MH⁺ 453

NMR (MeOH-d4 400 MHz; δ) 8.2 (1H, d, CH aromatic) 7.85 (1H, d, CH aromatic) 7.5 (1H, t, CH aromatic) 7.35 (1H, s, CH thiazole) 7.2 (1H, t, CH aromatic) 4.15 (2H, d, OCH₂) 3.75 (2H, d, CH₂ piperidine) 3.55 (2H, t, CH₂) 3.35(2H, t, CH₂) 3.1 (2H, t, CH₂ piperdine) 3.05 (3H, s, CH₃) 2.65 (3H, s, CH₃) 2.1 (3H, m, CH₂+CH piperidine) 1.7 (2H, q, CH₂ piperidine)

### Example 51

### (4-Fluoropiperidin-4-yl)methyl 2-(4-methyl-1,3-thiazol-2-yl)phenylcarbamate

A solution of benzyl 4-fluoro-4-[2-({[2-(4methyl-1,3-thiazol-2-yl)phenyl]amino}oxy)-2-oxoethyl]piperidine-1-carboxylate (100 mg) in ethanol was hydrogenolysed over palladium catalyst (10%; 50 mg) over 2 h. The catalyst was filtered off and the filtrate evaporated to give the title compound as a colourless solid (43 mg)

LC/MS ESI R_{T} 2.55 mins, MH⁺ 350

NMR (CDCl₃ 400 MHz; δ) 12.1 (1H, br S NH), 8.36 (1H, br S, aromatic CH) 7.75 (1H, dd, aromatic CH) 7.40 (1H, ddd, aromatic CH) 7.09 (1H, ddd, aromatic CH) 6.90 (1H, d, aromatic CH) 4.34 (2H, d, [J 21 Hz],CH₂) 3.44 (2H, br d, CH₂ EQ) 3.21 (2H, m, CH₂ AX) 2.50 (3H, s, CH₃) 2.28-2.08 (4H, m, 2×CH₂) 1.80 (3H, m, CH₂+CH) 1.40 (2H, br q, CH₂).

### Example 52

### [(2alpha,6beta)-1-benzyl-2,6-diethylpiperidin-4-yl]methyl 2-(4-methyl-1,3-thiazol-2-yl)phenylcarbamate

Triphosgene (39 mg) was added to a solution of [(2alpha,6beta)-1-benzyl-2,6-dimethylpiperidin-4-yl]methanol (61 mg) and diisopropylethylamine (0.1 ml) in dry THF (5 ml) at room temperature under nitrogen. The mixture was stirred for 2 h, then a solution of 2-(4-methyl-1,3-thiazol-2-yl)aniline (50 mg) in dry THF (1 ml) was added dropwise and the mixture stired for 16 h. The solvent was evaporated and the residue purified by chromatography on silica. Elution with dichloromethane/ethanol/ammonia 400:8:1 gave the title compound as a colourless foam (31 mg)

NMR (CDCl₃ 400 MHz; δ) 11.90 (1H, br s NH), 8.42 (1H, br d, aromatic CH) 7.72 (1H, dd, aromatic CH) 7.41-7.18 (6H, m, aromatic 6×CH) 7.04 (1H, br t, aromatic CH) 6.85 (1H, s, aromatic CH) 4.01 (2H, d, CH₂) 3.93,3.44 (2H, 2×d, CH₂) 3.02 (1H, in, CH) 2.88 (1H, m, CH) 2.52δ (3H, s, CH₃) 2.14 (1H, m, CH) 1.70 (1H, br d, CH EQ) 1.55-1.46 (2H, m, CH₂) 1.15 (1H, t, CH AX) 1.09 (3H, d, CH₃) 1.00 (3H, d, CH₃).

LC/MS ESI R_{T} 2.94 mins MH⁺ 450

### Example53

### [(2α,6β)-2,6-Dimethylpiperidin-4-yl]methyl 2-(4-methyl-1,3-thiazol-2-yl)phenylcarbamate

A solution of [(2alpha,6beta)-1-benzyl-2,6-dimethylpiperidin-4-yl]methyl 2-(4-methyl-1,3-thiazol-2-yl)phenylcarbamate (31 mg) in ethanol (10 ml) was hydrogenolysed over palladium oxide (10% on carbon; 10 mg) for 16 h. The catalyst was filtered off and the filtrate evaporated to give the title compound as a mixture of enantiomers (2 mg)

LC/MS ESI R_{T} 2.8 mins MH⁺ 360

### Example 54

### [(2alpha,6beta)-2,6-dimethylpiperidin-4-yl]methyl 2-(4-methyl-1,3-thiazol-2-yl)phenylcarbamate hydrochloride isomer 1

Ethereal HCl (1M; 2 ml) was added to a solution of tert-butyl (2R,6R)-2,6-dimethyl-4-{[({[2-(4-methyl-1,3-thiazol-2-yl)phenyl]amino}carbonyl)oxy]methyl}piperidine-1-carboxylate isomer 1 in methanol (2 ml) containing dichloromethane (0.5 ml) and the mixture st at room temperature for 18 h. The solvent was evaporated to give the title compound as a colourless solid (65 mg)

LC/MS ESI R_{T} 2.80 mins MH⁺ 360

Sample resolved on CHIRALCEL OD-H

Manufacturer DIACEL CHEMICAL INDUSTRIES LTD

Column size 0.46 cm I.D.×25 cm

Column no. ODHOCE-IF029

Eluent 10% Ethanol/Heptane

Flowrate 1 ml/min

Temp. RT

Wavelength 215 nm

Injection volume 15 ul

Retention time 10.69 mins

### Example 55

### [(2alpha,6beta)-2,6-dimethylpiperidin-4-yl]methyl 2-(4-methyl-1,3-thiazol-2-yl)phenylcarbamate hydrochloride isomer 2

Ethereal HCl (1M; 2 ml) was added to a solution of tert-butyl (2S,6S)-2,6-dimethyl-4-{[({[2-(4-methyl-1,3-thiazol-2-yl)phenyl]amino}carbonyl)oxy]methyl}piperidine-1-carboxylate isomer 2 (40 mg) in methanol (2 ml) containing dichloromethane (0.5 ml) and the mixture stirred at room temperature for 18 h. The solvent was evaporated to give the title compound as a colourless solid (37 mg)

LC/MS ESI R_{T} 2.81 mins MH⁺ 360

5048-Sample resolved on CHIRALCEL OD-H

Manufacturer DIACEL CHEMICAL INDUSTRIES LTD

Column size 0.46 cm LD.×25 cm

Column no. ODHOCE-IF029

Eluent 10% Ethanol/Heptane

Flowrate 1 ml/min

Temp. RT

Wavelength 215 nm

Injection volume 15 ul

Retention time=12.21 mins

### Example 56

[(2alpha,4beta,6alpha)-1-benzyl-2,6-dimethylpiperidin-4-yl]methyl 2-(4-methyl-1,3-thiazol-2-yl)phenylcarbamate isomer 1

Triphosgene (64 mg) was added to a solution of [(2alpha,4beta,6alpha)-1-benzyl-2,6-dimethylpiperidin-4-yl]methanol isomer 2 (100 mg) and diisopropylethylamine (0.15 ml) in dry THF (5 ml) at room temperature under nitrogen. The mixture was stirred for 2 h, then a solution of 2-(4-methyl-1,3-thiazol-2-yl)aniline (81 mg) in dry THF (1 ml) was added dropwise and the mixture stirred for 16 h. The yellow suspension was partitioned betweem water (10 ml) and ethyl acetate (3×10 ml) and the combined organic extracts dried (MgSO4). The solvent was evaporated and the residue purified by chromatography on silica. Elution with dichloromethane/ethanol/ammonia 400:8:1 gave the title compound as a colourless solid (61 mg)

LC/MS ESI R_{T} 3.04 mins MH⁺ 450

Tlc SiO₂ (Dichloromethane/ethanol/ammonia 200:8:1) R_{f} 0.2

### Example 57

### [(2alpha,4alpha,6alpha)-1-benzyl-2,6-dimethylpiperidin-4-yl]methyl 2-(4-methyl-1,3-thiazol-2-yl)phenylcarbamate isomer 2

Triphosgene (43 mg) was added to a solution of [(2alpha,4alpha,6alpha)-1-benzyl-2,6-dimethylpiperidin-4-yl]methanol isomer 2 (B) (67 mg) and diisopropylethylamine (0.10 ml) in dry TV (5 ml) at room temperature under nitrogen. The mixture was stirred for 2 h, then a solution of 2-(4-methyl-1,3-thiazol-2-yl)anline (54 mg) in dry THF (1 ml) was added dropwise and the mixture stirred for 16 h. The yellow suspension was partitioned between water (10 ml) and ethyl acetate (3×10 ml) and the combined organic extracts dried (MgSO₄). The solvent was evaporated and the residue purified by chromatography on silica. Elution with dichloromethane/ethanol/ammonia 300:8:1 gave the title compound as a colourless solid (76 mg)

LC/MS ESI R_{T} 3.07 mins MH⁺ 450

Tlc SiO₂ (Dichloromethane/ethanol/ammonia 200:8:1) R_{f} 0.18

### Example 58

[(2alpha,4beta,6alpha)-2,6-dimethylpiperidin-4-yl]methyl 2-(4-methyl-1,3-thiazol-2-yl)phenylcarbamate isomer 2

A solution of [(2alpha,4beta,6alpha)-1-benzyl-2,6-dimethylpiperidin-4-yl]methyl 2-(4-methyl-1,3-thiazol-2-yl)phenylcarbamate isomer 1 (61 mg) in ethanol (4 ml) was hydrogenolysed over palladium (10 mg) for 16 h. The catalyst was filtered off and the filtrate evaporated to give the title compound as a colourless solid (43.8 mg)

LC/MS ESI R_{T} 2.80 mins MH⁺ 360

NMR (CDCl₃/MeOD 400 MHz; δ) 8.37 (1H, br d, aromatic CH), 7.74 (1H, dd, aromatic CH) 7.39 (1H, ddd, aromatic CH) 7.08 (1H, ddd, aromatic CH) 6.90 (1H, s, aromatic CH) 4.28 (2H, d, CH₂) 3.30 (2H, m, 2×CH) 2.51 (3H, s, CH₃) 2.00-1.85 (4H, m, 2×CH₂) 1.47 (6H, d, 2×CH₃)

### Example 59

### [(2alpha,4alpha,6alpha)-2,6-dimethylpiperidin-4-yl]methyl 2-(methyl-1,3-thiazol-2-yl)phenylcarbamate isomer 1

A solution of [(2alpha,4alpha,6alpha)-1-benzyl-2,6-dimethylpiperidin-4-yl]methyl 2-(4-methyl-1,3-thiazol-2-yl)phenylcarbamate isomer 2 (76 mg) in ethanol (4 ml) was hydrogenolysed over palladium (10 mg) for 16 h. The catalyst was filtered off and the filtrate evaporated. The residue was purified by chromatography on silica. Elution with dichloromethane/ethanol/ammonia 100:8:1 gave the title compound as a colourless solid (23.5 mg)

LC/MS ESI R_{T} 2.77 mins MH⁺ 360

NMR (CDCl₃ 400 MHz; δ) 11.80 (1H, br s, NH) 8.42 (1H, br d, aromatic CH), 7.72 (1H, dd, aromatic CH) 7.38 (1H, ddd, aromatic CH) 7.04 (1H, ddd, aromatic CH) 6.86 (1H, br s, aromatic CH) 4.02 (2H, d, CH₂) 2.73 (2H, m, 2×CH) 2.52 (3H, s, CH₃) 1.91 (1H, m, CH) 1.76 (2H, br d, CH₂ EQ) 1.10 (6H, d, 2×CH₃) 0.82 (2H, br q, CH₂ AX)

### Intermediate 131

### tert-Butyl 4-{[({[4-(4,4,5,5-tetrametyl-[1,3,2]dioxaborolan-2-yl) phenyl]amino}carbonyl)oxy]methyl}iperidine-1-carboxylate

A mixture of tert-butyl 4-(hydroxymethyl)piperidine-1-carboxylate (147.5 mg) and diisopropylethylamine (0.119 ml) in dry THF (1.5 ml) was added dropwise to a solution of triphosgene (67 mg) in dry THF (1.5 ml) at 0-5° C. under nitrogen. The mixture was stirred for 1.5 h, then a solution of 4-(4,4,5,5-tetramethyl-[1,3,2]dioxaborolan-2-yl)-phenylamine (150 mg) in dry THF (1.5 ml) was added dropwise. The mixture was stirred for 16 h at room temperature. Water (10 ml) followed with ethyl acetate (5 ml) were added to the reaction. The aqueous phase was extracted with ethyl acetate (5 ml). The combined organics were washed with brine (10 ml) and dried (Na₂SO₄). The solvent was evaporated and the residue purified by Biotage Flash™ on silica. Elution with dichloromethane followed by ethylacetate gave the title compoundas a pale yellow powder (280 mg)

LC/MS ESI R_{T} 2.69 mins MH⁺ 460.4

Tlc SiO₂ (1:1 Hexane:Ethyl Acetate) Rf 0.75

### Intermediate 132

### tert-Butyl 4-{[({[3-(4,4,5,5-tetramethyl-[1,3,2]dioxaborolan-2-yl) phenyl]amino}carbonyl)oxy]methyl}piperidine-1-carboxylate

A mixture of tert-butyl 4-(hydroxymethyl)piperidine-1-carboxylate (235 mg) and diisopropylethylamine (0.19 ml) in dry TBF (1.5 ml) was added dropwise to a solution of triphosgene (108 mg) in dry THF (2.0 ml) at 0-5° C. under nitrogen. The mixture was stirred for 1.5 h, then a solution of 3-(4,4,5,5-Tetramethyl-[1,3,2]dioxaborolan-2-yl)-phenylamine (239 mg) in dry THF (1.5 ml) was added dropwise. The mixture was stirred for 16 h atroom temperature. Water (10 ml) followed with ethyl acetate (5 ml) were added to the reaction. The aqueous phase was extracted with ethyl acetate (5 ml). The combined organics were washed with brine (10 ml) and dried (Na₂SO₄). The solvent was evaporated and the residue purified by Biotage Flash™ on silica. Elution with dichloromethane followed by ethylacetate gave the title compound as a pale yellow powder (501 mg)

LC/MS ESI R_{T} 2.61 mins M⁺ 460.4

Tlc SiO₂ (1:1 Hexane:Ethyl Acetate) Rf 0.73

### Example 60

### tert-Butyl 4-{[({[4-(4-chloro-1,3-thiazol-2-yl) phenyl]amino}carbonyl)oxy]methyl}piperidine-1-carboxylate

Amixture of tert-Butyl 4-{[({[4-(4,4,5,5-tetrametyl-[1,3,2]dioxaborolan-2-yl) phenyl]amino}carbonyl)oxy]methyl}piperidine-1-carboxylate (400 mg) and 2,4-dichloro-1,3-thaizol (134 mg) were dissovled in ethylene glycol dimethyl ether (8 ml) and 2M sodium bicarbanate in water (3 ml). Nitrogen was bubbled through for more than 10 mins before tetrakis(triphenyl pbosphine) palladium (0) (201 mg)was added in one portion. The reaction mixture was heated at 80 degree for 10 hours. Water (15 ml) followed with ethyl acetate (20 ml) were added to the reaction. The aqueous phase was extracted with ethyl acetate (15 ml). The combined organics were washed with brine (10 ml) and dried (Na₂SO₄). The solvent was evaporated and the residue purified by Biotage Flash™ on silica Elution with dichloromethane followed by ethylacetate gave the title compound as a pale yellow powder (160 mg)

LC/MS ESI R_{T} 2.75 mins M⁺ 452.2

### Example 61

### tert-Butyl 4-{[({[3-(4-chloro-1,3-tiazol-2-yl) phenyl]amino}carbonyl)oxy]methyl}ipeRndine-1-carboxylate

A mixture of tert-Butyl 4-{[({[3-(4,4,5,5-tetrametyl-[1,3,2]dioxaborolan-2-yl) phenyl]amino}carbonyl)oxy]methyl}piperidine-1-carboxylate (500 mg) and 2,4-dichloro-1,3-thaizol (168 mg) were dissovled in ethylene glycol dimethyl ether (16 ml) and 2M sodium bicarbanate in water (8 ml). Nitrogen was bubbled through for more than 10 mins before tetrakis(triphenyl phosphine) palladium (0) (251 mg) was added in one portion. The reaction mixture was heated at 80 degree for 10 hours. Water (15 ml) followed with ethyl acetate (20 ml) were added to the reaction. The aqueous phase was extracted with ethyl acetate (15 ml). The combined organics were washed with brine (10 ml) and dried (Na₂SO₄). The Solvent was evaporated and the residue purified by Biotage Flash™ on silica Elution with dichioromethane followed by ethylacetate gave the title compound as a pale yellow powder (300 mg)

LC/MS ESI R_{T} 2.77 mins M⁺ 452.2

### Example 62

### Pideridin-4-ylmethyl 4-(4-chloro-1,3-thiazol-2-yl)phenylcarbamate hydrochloride

A solution of tert-butyl 4-{[({[4-(4-chloro-1,3-thiazol-2-yl) phenyl]amino}carbonyl)oxy]methyl}piperidine-1-carboxylate (120 mg) in methanol (10 ml) was treated with 4M hydrogen chloride in dioxane (1 ml). The reaction mixture was stirred at room temperature for 16 h. The mixture was then concentrated and the resultant residue was triturated in 5:1, ether/ethyl acetate to give the title compound as a yellow powder (100 mg)

LC/MS ESI R_{T} 1.58 mins M⁺ 352.2

### Example 63

### Piperidin-4-ylmethyl 3-(4-chloro-1,3-thiazol-2-yl)phenylcarbamate hydrochloride

A solution of tert-butyl 4-{[({[3-(4chloro-1,3-thiazol-2-yl) phenyl]amino}carbonyl)oxy]methyl}piperidine-1-carboxylate (300 mg) in methanol (10 ml) was treated with 4M hydrogen chloride in dioxane (3 ml) . The reaction mixture was stirred at room temperature for 16 h. The reaction mixture was filtered and washed with methylene chloride and methanol to give the title compound as a yellow powder (110 mg)

LC/MS ESI R_{T} 1.40 mins M⁺ 352.2

### Example 64

### 1-cyclohexylmethyl-piperidin-4-ylmethyl 4-(4-chloro-1,3-thiazol-2-yl)phenylcarbamate

A solution piperidin-4-ylmethyl 4(4-chloro-1,3-thiazol-2-yl)phenylcarbamate (30 mg) in methylene chloride (10 ml)was treated with cyclohexanecarbaldehyde (0.01 ml) at 0 degree and stirred at 0 degree for half an hour before sodium triacetoxyborohydride (27 mg) was added in one portion. The reaction mixture was allowed to warm to room temperature slowly and stirred overnight. Methylene chloride (10 ml) was added to the eaction followed by satd NaHCO₃ (aq) (10 ml). The aqueous phase was extracted with thyl acetate (15 ml×3). The combined organics were washed with brine (10 ml) and ried (Na₂SO₄). The solvent was evaporated to give the title compound as a white powder (21 mg).

LC/MS ESI R_{T} 1.73 mins M⁺ 448.2

### Example 65

### 1-cyclohexylmethyl-piperidinylmethyl 3-(4-chloro-1,3-thiazol-2-yl)phenylcarbamate

A solution piperidinylmethyl 3-(4-chloro-1,3-thiazol-2-yl)phenylcarbamate (60 mg) in methylene chloride (20 ml) was treated with cyclohexanecarbaldehyde (0.01 ml) at 0 degree and stirred at 0 degree for half an hour before sodium triacetoxyborohydride (27 mg) was added in one portion. The reaction mixture was allowed to warm to room temperature slowly and stirred overnight. Methylene chloride (10 ml) was added to the reaction followed by satd NaHCO₃ (aq) (10 ml). The aqueous phase was extracted with ethyl acetate (15 ml×3). The combined organics were washed with brine (10 ml) and dried (Na₂SO₄). The solvent was evaporated to give the title compound as a white powder (41 mg).

LC/MS ESI R_{T} 1.97 mins M⁺ 448.2

### Example 66

### 4-[4-(4-Chloro-thiazol-2-yl)-phenylcarbamoyloxymethyl]-1-cyclohexylmethyl-1-methyl-piperidinium iodide

1-cyclohexylmethyl-piperidin-4-ylmethyl 4-(4-chloro-1,3-thiazol-2-yl)phenylcarbamate (20 mg) was dissolved in a mixture of methanol (5 ml) and methylene chloride (10 ml). Methyl iodide (1 ml) was added at room temperature followed by NaCO₃ (50 mg). The reaction mixture was filtered through a pad of celite after stirring overnight at room temperature to afford the title compound as a white powder (13 mg).

LC/MS ESI R_{T} 1.82 mins M⁺ 462.4

### Example 67

### 4-[3-(4-Chloro-thiazol-2-yl)-phenylcarbamoyloxymethyl]-1-cyclohexylmethyl-1-methyl-piperidinium iodide

1-cyclohexylmethyl-piperidin-4-ylmethyl 3-(4-chloro-1,3-thiazol-2-yl)phenylcarbarate (18 mg) was dissolved in a mixture of methanol (5 ml) and methylene chloride (10 ml). Methyl iodide (1 ml) was added at room temperature followed by NaCO₃ (50 mg). The reaction mixture was filtered through a pad of celite after stirring overnight at room temperature to afford the title compound as a white powder (10 mg).

LC/MS ESI R_{T} 1.95 mins M⁺ 462.4

### Example 68

### 4-[4-(4-Chloro-thiazol-2-yl)-phenylcarbamoylox&methyl]-1,1-dimethyl-piperidinium

A solution piperidin-4-ylmethyl 4-(4-chloro-1,3-thiazol-2-yl)phenylcarbamate (20 mg) in methylene chloride (10 ml) and methanal (5 ml) was treated with methyl iodide (1 ml) at room temperature followed by NaCO₃ (50 mg). The reaction mixture was filtered through a pad of celite after stirring overnight at room temperature to afford the title compound as a white powder (11 mg).

LC/MS ESI R_{T} 1.48 mins M⁺ 380.2

### Example 69

### 4-[3-(4-Chloro-thiazol-2-yl)-phenylcarbamoyloxymethyl]-1,1-dimethyl-piperidinium

A solution piperidinylmethyl 3-(4-chloro-1,3-thiazol-2-yl)phenylcarbamate (40 mg) in methylene chloride (10 ml) and methanal (5 ml) was treated with methyl iodide (1 ml) at room temperature followed by NaCO₃ (50 mg). The reaction mixture was filtered through a pad of celite after stirring overnight at room temperature to afford the title compound as a white powder (17 mg).

LC/MS ESI R_{T} 1.55 mins M⁺ 380.4

### BIOLOGICAL EXAMPLES

The inhibitory effects of compounds at the M₃ mAChR of the present invention are determined by the following in vitro and in vivo functional assays:

Analysis of Inhibition of Receptor Activation by Calcium Mobilization:

Stimulation of mAChRs expressed on CHO cells were analyzed by monitoring eceptor-activated calcium mobilization as previously described (4). CHO cells stably expressing M3 mAChRs were plated in 96 well black wall/clear bottom plates. After 18 to 24 hours, media was aspirated and replaced with 100 μl of load media (EMEM with Earl's salts, 0.1% RIA-grade BSA (Sigma, St. Louis Mo.), and 4 μM Fluo-3-acetoxymethyl ester fluorescent indicator dye (Fluo-3 AM, Molecular Probes, Eugene, Oreg.) and inubated 1 hr at 37° C. The dye-contag media was then aspirated, replaced with fresh media (without Fluo-3 AM), and cells were incubated for 10 minutes at 37° C. Cells were then washed 3 times and incubated for 10 minutes at 37° C. in 100 μl of assay buffer (0.1% gelatin (Sigma), 120 mM NaCl, 4.6 mM KCl, 1 mM KH₂PO₄, 25 mM NaH CO₃, 1.0 mM CaCl₂, 1.1 mM MgCl₂, 11 mM glucose, 20 mM HEPES (pH 7.4)). 50 μl of compound (1×10⁻¹¹-1×10⁻⁵ M fmal in the assay) was added and the plates were incubated for 10 min. at 37° C. Plates were then placed into a fluorescent light intensity plate reader (FLIPR, Molecular Probes) where the dye loaded cells were exposed to excitation light (488 nm) from a 6 watt argon laser. Cells were activated by adding 50 μl of acetylcholine (0.1-10 nM final), prepared in buffer containing 0.1% BSA, at a rate of 50 μl/sec. Calcium mobilization, monitored as change in cytosolic calcium concentration, was measured as change in 566 nm emission intensity. The change in emission intensity is directly related to cytosolic calcium levels (5). The emitted fluorescence from all 96 wells is measured simultaneously using a cooled CCD camera Data points are collected every second. This data was then plotting and analyzed using GraphPad PRISM software.

Methacholine-Induced Bronchoconstriction

Airway responsiveness to methacholine was determined in awake, unrestrained BalbC mice (n=6 each group). Barometric plethysmography was used to measure enhanced pause (Penh), a unitless measure that has been shown to correlate with the changes in airway resistance that occur during bronchial challenge with methacholine (2). Mice were pretreated with 50 μl of compound (0.003-10 μg/mouse) in 50 μl of vehicle (10% DMSO) intranasally, i.v., i.p. or p.o, and were then placed in the plethysmography chamber. Once in the chamber, the mice were allowed to equilibrate for 10 min before ta g a baseline Penh measurement for 5 minutes. Mice were then challenged with an aerosol of methacholine (10 mg/ml) for 2 minutes. Penh was recorded continuously for 7 min starting at the inception of the methacholine aerosol, and continuing for 5 minutes afterward. Data for each mouse were analyzed and plotted by using GraphPad PRISM software.

The present compounds are useful for treating a variety of indications, including but not limited to respiratory-tract disorders such as chronic obstructive lung disease, chronic bronchitis, asthma, chronic respiratory obstruction, pulmonary fibrosis, pulmonary emphysema, and allergic rhinitis; gastrointestinal-tract disorders such as irritable bowel syndrome, spasmodic colitis, gastroduodenal ulcers, gastrointestinal convulsions or hyperanakinesia, diverticulitis, pain accompanying spasms of gastrointestinal smooth musculature; urinary-tract disorders accompanying micturition disorders including neurogenic pollakisuria, neurogenic bladder, nocturnal enuresis, psychosomatic bladder, incontinence associated with bladder spasms or chronic cystitis, urinary urgency or pollalduria, and motion sickness.

Methods of administering the present compounds will be readily apparent to the skilled artisan.

Dry powder compositions for topical delivery to the lung by inhalation may, for example, be presented in capsules and cartridges of for example gelatine, or blisters of for example laminated aluminium foil, for use in an inhaler or insufflator. Formulations generally contain a powder mix for inhalation of the compound of the invention and a suitable powder base (carrier substance) such as lactose or starch. Use of lactose is preferred Each capsule or cartridge may generally contain between 20 μg-10 mg of the compound of formula (I) optionally in combination with another therapeutically active ingredient. Alternatively, the compound of the invention may be presented without excipients.

Suitably, the medicament dispenser is of a type selected from the group consisting of a reservoir dry powder inhaler (RDPI), a multi-dose dry powder inhaler (MDPI), and a metered dose inhaler (MDI).

By reservoir dry powder inhaler (RDPI) it is meant an inhaler having a reservoir form pack suitable for comprising multiple (un-metered doses) of medicament in dry powder form and including means for metering medicament dose from the reservoir to delivery position. The metering means may for example comprise a metering cup, hich is movable from a first position where the cup may be filled with medicament rom the reservoir to a second position where the metered medicament dose is made vailable to the patient for inhalation.

By multi-dose dry powder inhaler (MDPI) is meant an inhaler suitable for ispensing medicament in dry powder form, wherein the medicament is comprised ithin a multi-dose pack containing (or otherwise carrying) multiple, define doses (or parts thereof) of medicament. In a preferred aspect, the carrier has a blister pack form, but it could also, for example, comprise a capsule-based pack form or a carrier onto which medicament has been applied by any suitable process including printing, painting and vacuum occlusion.

The formulation can be pre-metered (eg as in Diskus, see GB 2242134 or Diskhaler, see GB 2178965, 2129691 and 2169265) or metered in use (eg as in Turbuhaler, see EP 69715). An example of a unit-dose device is Rotahaler (see GB 2064336). The Diskus inhalation device comprises an elongate strip formed from a base sheet having a plurality of recesses spaced along its length and a lid sheet hermetically but peelably sealed thereto to define a plurality of containers, each container having therein an inhalable formulation containing a compound of formula (I) preferably combined with lactose. Preferably, the strip is sufficiently flexible to be wound into a roll. The lid sheet and base sheet will preferably have leading end portions which are not sealed to one another and at least one of the said leading end portions is constructed to be attached to a winding means. Also, preferably the hermetic seal between the base and lid sheets extends over their whole width. The lid sheet may preferably be peeled from the base sheet in a longitudinal direction from a first end of the said base sheet.

In one aspect, the multi-dose pack is a blister pack comprising multiple blisters for containment of medicament in dry powder form. The blisters are typically arranged in regular fashion for ease of release of medicament therefrom.

In one aspect, the multi-dose blister pack comprises plural blisters arranged in generally circular fashion on a disc-form blister pack. In another aspect, the multi-dose blister pack is elongate in form, for example comprising a strip or a tape.

Preferably, the multi-dose blister pack is defined between two members eelably secured to one another. U.S. Pat. Nos. 5,860,419, 5,873,360 and 5,590,645 escribe medicament packs of this general type. In this aspect, the device is usually rovided with an opening station comprising peeling means for peeling the members apart to access each medicament dose. Suitably, the device is adapted for use where the peelable members are elongate sheets which define a plurality of medicament containers spaced along the length thereof, the device being provided with indexing means for indexing each container in turn. More preferably, the device is adapted for use where one of the sheets is a base sheet having a plurality of pockets therein, and the other of the sheets is a lid sheet, each pocket and the adjacent part of the lid sheet definng a respective one of the containers, the device comprising driving means for puling the lid sheet and base sheet apart at the opening station.

By metered dose inhaler (MDI) it is meant a medicament dispenser suitable for dispensing medicament in aerosol form, wherein the medicament is comprised in an aerosol container suitable for containing a propellant-based aerosol medicament formulation. The aerosol container is typically provided with a metering valve, for example a slide valve, for release of the aerosol form medicament foimulation to the patient. The aerosol container is generally designed to deliver a predetermined dose of medicament upon each actuation by means of the valve, which can be opened either by depressing the valve while the container is held stationary or by depressing the container while the valve is held stationary.

Where the medicament container is an aerosol container, the valve typically comprises a valve.body having an inlet port through which a medicament aerosol formulation may enter said valve body, an outlet port through which the aerosol may exit the valve body and an open/close mechanism by means of which flow through said outlet port is controllable.

The valve may be a slide valve wherein the open/close mechanism comprises a sealing ring and receivable by the sealing ring a valve stem having a dispensing passage, the valve stem being slidably movable within the ring from a valve-closed to a valve-open position in which the interior of the valve body is in communication with the exterior of the valve body via the dispensing passage.

Typically, the valve is a metering valve. The metering volumes are typically from 10 to 100 μl, such as 25 μl, 50 μl or 63 μl. Suitably, the valve body defines a metering chamber for metering an amount of medicament formulation and an open/close mechanism by means of which the flow through the inlet port to the metering chamber is controllable. Preferably, the valve body has a sampling chamber in communication with the metering chamber via a second inlet port, said inlet port being controllable by means of an open/close mechanism thereby regulating the flow of medicament formulation into the metering chamber.

The valve may also comprise a ‘free flow aerosol valve’ having a chamber and a valve stem extending into the chamber and movable relative to the chamber between dispensing and non-dispensing positions. The valve stem has a configuration and the chamber has an internal configuration such that a metered volume is defined therebetween and such that during movement between is nonispensing and dispensing positions the valve stem sequentially: (i) allows free flow of aerosol formulation into the chamber, (ii) defines a closed metered volume for pressurized aerosol formulation between the external surface of the valve stem and internal surface of the chamber, and (iii) moves with the closed metered volume within the chamber without decreasing the volume of the closed metered volume until the metered volume communicates with an outlet passage thereby allowing dispensing of the metered volume of pressurized aerosol formulation. A valve of this type is described in U.S. Pat. No. 5,772,085. Additionally, intra-nasal delivery of the present compounds is effective.

To formulate an effective pharmaceutical nasal composition, the medicament must be delivered readily to all portions of the nasal cavities (the target tissues) where it performs its pharmacological function. Additionally, the medicament should remain in contact with the target tissues for relatively long periods of time. The longer the medicament remains in contact with the target tissues, the medicament must be capable of resisting those forces in the nasal passages that function to remove particles from the nose. Such forces, referred to as ‘mucociliary clearance’, are recognised as being extremely effective in removing particles from the nose in a rapid manner, for example, within 10-30 minutes from the time the particles enter the nose.

Other desired characteristics of a nasal composition are that it must not contain ingredients which cause the user discomfort, that it has satisfactory stability and shelf-life properties, and that it does not include constituents that are considered to be detrimental to the environment, for example ozone depletors.

A suitable dosing regime for the formulation of the present invention when administered to the nose would be for the patient to inhale deeply subsequent to the nasal cavity being cleared. During inhalation the formulation would be applied to one nostril while the other is manually compressed. This procedure would then be repeated for the other nostril.

A preferable means for applying the formulation of the present invention to the nasal passages is by use of a pre-compression pump. Most preferably, the pre-compression pump will be a VP7 model manufactured by Valois SA. Such a pump is beneficial as it will ensure that the formulation is not released until a sufficient force has been applied, otherwise smaller doses may be applied. Another advantage of the pre-compression pump is that atomisation of the spray is ensured as it will not release the formulation until the threshold pressure for effectively atomising the spray has been achieved. Typically, the VP7 model may be used with a bottle capable of holding 10-50 ml of a formulation. Each spray will typically deliver 50-100 μl of such a formulation, therefore, the VP7 model is capable of providing at least 100 metered doses.

### EXAMPLES OF NASAL FORMULATIONS

### Example 1

### Nasal Formulation Containing Active

A formulation for intranasal delivery was prepared with ingredients as follows:

| | to 100% | |
|---|---|---|
| Active | 0.1% | w/w |
| Polysorbate 80 | 0.025% | w/w |
| Avicel RC591 | 1.5% | w/w |
| Dextrose | 5.0% | w/w |
| BKC | 0.015% | w/w |
| EDTA | 0.015% | w/w |

| | |
|---|---|
| water | to 100% |

in a total amount suitable for 120 actuations and the formulation was filled into a bottle fitted with a metering valve adapted to dispense 50 or 100 μl per actuation. The device was fitted into a nasal actuator (Valois).

### Example 2

### Nasal Formulation Containing Active

A formulation for intranasal delivery was prepared with ingredients as follows:

| | | |
|---|---|---|
| Active | 0.005% | w/w |
| Tyloxapol | 2% | w/w |
| dextrose | 5% | w/w |
| BKC | 0.015% | w/w |
| EDTA | 0.015% | w/w |

| | |
|---|---|
| water | to 100% |

in a total amount suitable for 120 actuations and the formulation was filled into a bottle (plastic or glass) fitted with a metering valve adapted to dispense 50 or 100 μl per actuation

The device was fitted into a nasal actuator (Valois, e.g. VP3, VP7 or VP7D)

### Example 3

### Nasal Formulation Containing Active

A formulation for intranasal delivery was prepared with ingredients as follows:

| | | |
|---|---|---|
| active | 0.05% | w/w |
| Triton X-100 | 5% | w/w |
| Dextrose | 4% | w/w |
| BKC | 0.015% | w/w |
| EDTA | 0.015% | w/w |

| | |
|---|---|
| water | to 100% |

in a total amount suitable for 120 actuations and the formulation was filled into a bottle fitted with a metering valve adapted to dispense 50 or 100 μl per actuation.

### Example 4

### Nasal Formulation Containing Active

A formulation for intranasal delivery was prepared with ingredients as follows:

| | | |
|---|---|---|
| active | 0.05% | w/w |
| Tyloxapol | 5% | w/w |
| dextrose | 5% | w/w |
| BKC | 0.015% | w/w |
| EDTA | 0.015% | w/w |

| | |
|---|---|
| water | to 100% |

in a total amount suitable for 120 actuations and the formulation was filled into a bottle fitted with a metering valve adapted to dispense 50 or 100 μl per actuation The device was fitted into a nasal actuator (Valois).

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

All publications, including but not limited to patents and patent applications, cited in this specification are herein incorporated by reference as if each individual publication were specifically and individually indicated to be incorporated by reference herein as though fully set forth.

The above description fuilly discloses the invention including preferred embodiments thereof Modifications and improvements of the embodiments specifically disclosed herein are within the scope of the following claims. Without further elaboration, it is believed that one skilled in the are can, using the preceding description, utilize the present invention to its fullest extent.; Therefore the Examples herein are to be construed as merely illustrative and not a llmitation of the scope of the present invention in any way. The embodiments of the invention in which an exclusive property or privilege is claimed are defined as follows.

1. A compound according to the formula: 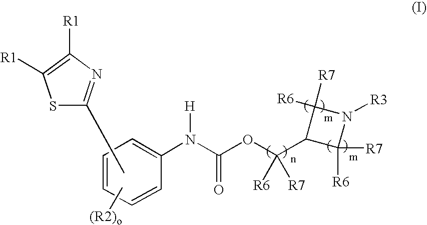
wherein:
the thiazole is ortho to the nitrogen;
R1 is selected from the group consisting of halogen, C₁₋₅alkly, CH₂F, CBF₂;
R2 is selected from the group consisting of hydrogen, C₁₋₅alkyl, aryl, halogen, hydroxy and alkoxy;
R3 is selected from the group consisting of hydrogen, C₁₋₅alkyl, cycloalkyl, cycloalkyl C₁₋₅ alkyl, C₂₋₄alkenyl, C₂₋₄alkenylaryl; cycloalkyl C₁₋₅ alkyl, and C₁₋₄alkylaryl, which may be optionally substituted independently by a substituent selected from the group consisting of halogen, nitro, halosubstituted C₁₋₄ alkyl, C₁₋₄ alkyl, amino, mono or di-C₁₋₄ alkyl substituted amine, ORₐ; C(O)Rₐ, NRₐC(O)ORₐ, OC(O)NR₆R₇, hydroxy, NR₉C(O)Rₐ, S(O)_{m′ R}ₐ, C(O)NR₆R₇, C(O)OH, C(O)ORₐ, S(O)₂NR₆R₇, and NHS(O)₂Rₐ;
R₆ and R₇ are selected from the group consisting of hydrogen, and C₁₋₄ alkyl, or R₆ and R₇ together form a 5 to 7 member ring which ring may optionally contain an additional heteroatom selected from oxygen, nitrogen or sulfur, and which ring may be optionally substituted;
n is 1 or 2; and independently
m is 1 or 2.

2. A compound according to claim 1 wherein:
the thiazole is ortho to the nitrogen;
R1 is selected from the group consisting of halogen, C₁₋₅alkyl, CH₂F, CHF₂;
R2 is selected from the group consisting of hydrogen, C₁₋₅aqkl, aryl, halogen, hydroxy and alkoxy;
R3 is selected from the group consisting of hydrogen, C₁₋₅alkyl, cycloalkyl, cycloalkyl C₁₋₅ alkyl, C₂₋₄alkenyl, C₂₋₄alkenylaryl; cycloalkyl C₁₋₅ alkyl, and C₁₋₄alkylaryl, which may be optionally substituted independently by a substituent selected from the group consisting of halogen, nitro, halosubstituted C₁₋₄ alkyl, C₁₋₄ alkyl, amlino, mono or di-C₁₋₄ alkyl substituted amine, ORₐ; C(O)Rₐ, NRₐC(O)ORₐ, OC(O)NR₆R₇, hydroxy, NR₉C(O)Rₐ, S(O)_{m′}Rₐ, C(O)NR₆R₇, C(O)OH, C(O)ORₐ, S(O)₂NR₆R₇, and NHS(O)₂Rₐ;
R₆ and R₇ are selected from the group consisting of hydrogen, and C₁₋₄ alkyl, or R₆ and R₇ together form a 5 to 7 member ring which ring may optionally contain an additionial heteroatom selected from oxygen, nitrogen or sulfuir, and which ring may be optionally substituted;
n is 1 or 2; and independently
m is 1 or 2.

3. A compound according to claim 2 selected from the group consisting of:
[2-(4-Methyl-thiazol-2-yl)-phenyl]-carbamnic acid piperidin-4-ylmethyl ester;
[2-(4-Ethyl-thiazol-2yl)-phenyl]-carbamic acid piperidin-4-ylmethyl ester;
{2-[4-(1,1-Difluoro-methyl)-thiazol-2-yl]-phenyl}-carbamic acid piperidin-4-ylmethyl ester;
(2-Thiazol-2-yl-phenyl)-carbamic acid piperidin-4-ylmethyl ester; compound with 2,2,2-trifluoroacetic acid;
[2-(4-Propyl-thiazol-2-yl)-phenyl]-carbamic acid piperidin-4-ylmethyl ester;
[2-(4-Methyl-thiazol-2-yl)-phenyl]-carbamic acid (2R,6R)-2,6-dimethyl-piperidin-4-ylmethyl ester;
[2-(4-Isopropyl-thiazol-2-yl)-phenyl]-carbamic acid piperidin-4-ylmethyl ester;
[2-(4-tert-Butyl-thiazol-2-yl)-phenyl]-carbamic acid piperidin-4-ylmethyl ester;
[2-(4-Bromo-thiazol-2-yl)-phenyl]-carbamic acid piperidin-4-ylmethyl ester;
[2-(4-Chloro-thiazol-2-yl)-phenyl]-carbamic acid piperidin-4-ylmethyl ester;
[2-(4-Isobutyl-thiazol-2-yl)-phenyl]-carbamic acid piperidin-4-ylmethyl ester;
[2-(4-Cyclopropylmethyl-thiazol-2-yl)-phenyl]-carbarnic acid piperidin-4-ylmethyl ester,
[2-(4-Cyclopropyl-thiazol-2-yl)-phenyl]-carbamic acid piperidin-4-ylmethyl ester;
[2-(4-Cyclobutyl-thiazol-2-yl)-phenyl]-carbamic acid piperidin-4-ylmethyl ester;
[2-(4-Trifluoromethyl-thiazol-2-yl)-phenyl]-carbamic acid piperidin-4-ylmethyl ester;
[2-(4-Fluoromethyl-thiazol-2-yl)-phenyl]-carbamic acid piperidin-4-ylmethyl ester;
{2-[4-(I1,1 -Difluoro-ethyl)-thiazol-2-yl]-phenyl}-carbamic acid piperidin-4-ylmethyl ester;
{2-[4-(2-Fluoro-ethyl)-thiazol-2-yl]-phenyl}-carbamic acid piperidin-4-ylmethyl ester;
{2-[4-(2,2-Difluoro-ethyl)-thiazol-2-yl]-phenyl}-carbamic acid piperidin-4-ylmethyl ester;
[2-(4-Methoxymethyl-thiazol-2-yl)-phenyl]-carbamic acid piperidin-4-ylmethyl ester;
[2-(4-Hydroxymethyl-thiazol-2-yl)-phenyl]-carbamic acid piperidin-4-ylmethyl ester;
{2-[4-(1-Hydroxy-ethyl)-thiazol-2-yl]-phenyl}-carbamic acid piperidin-4-ylmethyl ester;
{2-[4-((R)-1-Hydroxy-ethyl)-thiazol-2-yl]-phenyl}-carbamic acid piperidin-4-ylmethyl ester;
{2-[4-(2-Hydroxy-ethyl)-thiazol-2-yl]-phenyl}-carbamic acid piperidin-4-ylmethyl ester;
[2-(4-Amino-thiazol-2-yl)-phenyl]-carbamic acid piperidin-4-ylmethyl ester;
[5-Fluoro-2-(4-methyl-thiazol-2-yl)-phenyl]-carbamic acid piperidin-4-ylmethyl ester;
[2-(4-Ethyl-thiazol-2-yl)-4-hydroxy-phenyl]-carbamic acid piperidin-4-ylmethyl ester;
[2-(4-Methyl-thiazol-2-yl)-phenyl]-carbamic acid (2R,6S)-2,6-dimethyl-piperidin-4-ylmethyl ester;
[2-(4-Methyl-thiazol-2-yl)-phenyl]-carbamic acid (2R,6S)-2,6-dimethyl-piperidin-4-ylmethyl ester;
[2-(4-Methyl-thiazol-2-yl)-phenyl]-carbamic acid (2R,6S)-1-benzyl-2,6-dimethyl-piperidin-4-ylmethyl ester;
[2-(4-Methyl-thiazol-2-yl)-phenyl]-carbamic acid (2R,6S)-1-benzyl-2,6-dimethyl-piperidin-4-ylmethyl ester;
[2-(4-Methyl-thiazol-2-yl)-phenyl]-carbamic acid (2R,6R)-2,6-dimethyl-piperidin-4-ylmethyl ester;
[2-(4-Methyl-thiazol-2-yl)-phenyl]-carbamic acid (2R,6R)-2,6-dimethyl-piperidin-4-ylmethyl ester;
[2-(4-Methyl-thiazol-2-yl)-phenyl]-carbamic acid (2R,6R)-1-benzyl-2,6-dimethyl-piperidin-4-ylmethyl ester;
[2-(4-Methyl-thiazol-2-yl)-phenyl]-carbamic acid 4-fluoro-piperidin-4-ylmethyl ester;
[2-(4-Methyl-thiazol-2-yl)-phenyl]-carbamic acid 1-butyl-piperidin-4-ylmethyl ester;
[2-(4-Methyl-5-methylcarbamoyl-thiazol-2-yl)-phenyl]-carbamic acid piperidin-4-ylmethyl ester;
[2-(5-Methyl-thiazol-2-yl)-phenyl]-carbamic acid piperidin4-ylmethyl ester;
[2-(4,5-Dimethyl-thiazol-2-yl)-phenyl]-carbamic acid piperidin-4-ylmethyl ester;
[2-(4-Acetyl-thiazol-2-yl)-phenyl]-carbamic acid piperidin4-ylmethyl ester;
{2-[4-(2-Benzyloxy-ethyl)-thiazol-2-yl]-phenyl}-carbamic acid piperidin-4-ylmethyl ester;
[2-(4-Methylcarbamoyl-thiazol-2-yl)-phenyl]-carbamic acid piperidin-4-ylmethyl ester;
2-[2-(Piperidin-4-ylmethoxycarbonylamino)-phenyl]-thiazole-4-carboxylic acid ethyl ester;
[2-(4-Dimethylaminomethyl-thiazol-2-yl)-phenyl]-carbamic acid piperidin-4-ylmethyl ester;
[2-(4-Phenyl-thiazol-2-yl)-phenyl]-carbaric acid piperidin-4-ylmethyl ester;
[2-(4-Thiophen-3-yl-thiazol-2-yl)-phenyl]-carbamic acid piperidin-4-ylmethyl ester;
[2-(4-Ethyl-thiazol-2-yl)-4-fluoro-phenyl]-carbamic acid piperidin-4-ylmethyl ester;
tert-Butyl 4-{[({[4-(4,4,5,5-tetrametyl-[1,3,2]dioxaborolan-2-yl) phenyl]amino}carbonyl)oxy]methyl}piperidine-1-carboxylate;
tert-Butyl 4-{[({[3-(4,4,5,5-tetrametyl-[1,3,2]dioxaborolan-2-yl) phenyl]amino}carbonyl)oxy]methyl}piperidine-1-carboxylate;
tert-Butyl 4-{[({[4-(4-chloro-1,3-thiazol-2-yl) phenyl]amino}carbonyl)oxy]methyl}piperidine-1-carboxylate;
tert-Butyl 4-{[({[3-(4-chloro-1,3-thiazol-2-yl) phenyl]amino}carbonyl)oxy]methyl}piperidine-1-carboxylate;
Piperidin-4-ylmethyl 4-(4-chloro-1,3-thiazol-2-yl)phenylcarbamate hydrochloride;
Piperidin-4-ylmethyl 3-(4-chloro-1,3-thiazol-2-yl)phenylcarbamate hydrochloride;
1-cyclohexylmethyl-piperidin-4-ylmethyl 4-(4-chloro-1,3-thiazol-2-yl)phenylcarbamate;
1-cyclohexylmethyl-piperidin-4-ylmethyl 3-(4-chloro-1,3-thiazol-2-yl)phenylcarbamate;
4-[4-(4-Chloro-thiazol-2-yl)-phenylcarbamoyloxymethyl]-1-cyclohexylmethyl-1-methyl-piperidinium iodide;
4-[3-(4-Chloro-thiazol-2-yl)-phenylcarbamoyloxymethyl]-1-cyclohexylmethyl-1-methyl-piperidinium iodide;
4-[4-(4-Chloro-thiazol-2-yl)-phenylcarbamoyloxymethyl]-1,1-dimethyl-piperidinium; and
4-[3-(4-Chloro-thiazol-2-yl)-phenylcarbamoyloxymethyl]-1,1-dimethyl-piperidinium;
or a pharmaceutically acceptable salt thereof.

4. A method according to claim 3 wherein the compound is selected from the group consisting of:
[2-(4-Bromo-thiazol-2-yl)-phenyl]-carbamic acid piperidin-4-ylmethyl ester;
[2-(4-Chloro-thiazol-2-yl)-phenyl]-carbamic acid piperidin-4-ylmethyl ester;
[2-(4-Methyl-thiazol-2-yl)-phenyl]-carbamic acid piperidin-4-ylmethyl ester;
{2-[4-(1,1-Difluoro-methyl)-thiazol-2-yl]-phenyl}-carbamic acid piperidin-4-ylmethyl ester; and
[2-(4-Fluoromethyl-thiazol-2-yl)-phenyl]-carbamic acid piperidin-4-ylmethyl ester.

5. A method of antagonizing the M₃ muscarinic acetylcholine receptor by administering to a subject in need thereof a safe and effective amount of a compound according to claim 1.

6. A method of treating a disease or disorder selected from the group consisting of chronic obstructive lung disease, chronic bronchitis, asthma, chronic respiratory obstruction, pulmonary fibrosis, pulmonary emphysema, and allergic rhinitis, irritable bowel syndrome, spasmodic colitis, gastroduodenal ulcers, gastrointestinal convulsions or hyperanakinesia, diverticulitis, pain accompanying spasms of gastrointestinal smooth musculature; urinary-tract disorders accompanying micturition disorders, neurogenic pollakisuria, neurogenic bladder, nocturnal enuresis, psychosomatic bladder, incontinence associated with bladder spasms or chronic cystitis, urinary urgency or pollakiuria, and motion sickness.

7. A pharmaceutical formulation comprising an active according to claim 1 and a suitable carrier.

8. A container containing a pharmaceutical formulation according to claim 1 fitted with a metering valve.

9. A device adapted for intranasal delivery of a pharmaceutical formulation comprising a container according to claim 8.
